(19) United States
(12) Patent Application Publication
(10) Pub. No.: US 2018/0291378 A1
(43) Pub. Date: Oct. 11, 2018

(54) METHODS AND COMPOSITIONS FOR PROMOTING HAIR GROWTH

(71) Applicant: THE TRUSTEES OF COLUMBIA UNIVERSITY IN THE CITY OF NEW YORK, NEW YORK, NY (US)

(72) Inventors: Angela Christiano, Mahwah, NJ (US); Raphael Clynes, West Nyack, NJ (US)

(21) Appl. No.: 15/571,806

(22) PCT Filed: May 9, 2016
(86) PCT No.: PCT/US16/31541

§ 371 Date: Nov. 3, 2017

Related U.S. Application Data
(60) Provisional application No. 62/157,959, filed on May 7, 2015.

(51) Int. Cl. C12N 15/113; A61Q 7/00; A61K 8/49; A61K 31/519; A61P 17/14; A61K 8/64

(52) U.S. Cl. CPC C12N 15/1136; A61Q 7/00; A61K 8/4953; A61K 31/519; C12N 2310/122; A61K 8/64; C12N 2310/11; C12N 2310/14; A61P 17/14

(57) ABSTRACT

The presently disclosed subject matter relates, in certain embodiments, to compositions and methods for the inhibition of the JAK-STAT pathway in order to induce hair growth. In certain embodiments, the presently disclosed subject matter relates to topical treatments with small molecule inhibitors of the JAK-STAT pathway to induce hair growth.

### CROSS-REFERENCE TO RELATED APPLICATIONS

This application claims priority to U.S. Provisional Patent Application Ser. No. 62/157,959, filed May 7, 2015, which is hereby incorporated by reference in its entirety.

### GRANT INFORMATION

This invention was made with government support under grant numbers R01AR056016 and R21AR061881 awarded by the National Institutes of Health. The government has certain rights in the invention.

### 1. INTRODUCTION

The presently disclosed subject matter relates, in certain embodiments, to compositions and methods for the inhibition of the JAK-STAT pathway in order to induce hair growth. In certain embodiments, the presently disclosed subject matter relates to topical treatments with small molecule inhibitors of the JAK-STAT pathway to induce hair growth.

### 2. BACKGROUND OF THE INVENTION

Several hair growth disorders are characterized by the inability to re-enter the growth phase of the hair cycle (anagen). This can be due to hair follicle (HF) miniaturization in the case of androgenetic alopecia, or due to immune dysfunction in the case of alopecia areata. While current pharmacologic therapy for androgenetic alopecia is primarily focused on the prevention of further hair loss, the elusive search for pharmacologic agents to restart the hair cycle has been largely unsatisfactory.

### 3. SUMMARY OF THE INVENTION

In certain embodiments, the present disclosure is directed to methods of inducing hair growth in a mammalian subject, wherein the method comprising administering to a hair follicle of the subject a therapeutically effective amount of a Jak1, a Jak2, a Jak3, a Tyk2, a STAT1, a STAT2, a STAT3, a STAT4, a STAT5a, a STAT5b, a STAT6, an OSM, a gp130, an LIFR, and/or an OSM-Rβ inhibitor.

In certain embodiments, the present disclosure is directed to methods of inducing hair growth in a mammalian subject, wherein the method comprising administering to a hair follicle of the subject a therapeutically effective amount of a Jak1, a Jak2, a Jak3, a Tyk2, a STAT1, a STAT2, a STAT3, a STAT4, a STAT5a, a STAT5b, a STAT6, an OSM, a gp130, an LIFR, and/or an OSM-Rβ inhibitor, and wherein said administration occurs when the hair follicle is in mid-telogen phase or late telogen phase.

In certain embodiments, the present disclosure is directed to methods of inducing hair growth in a mammalian subject, wherein the method comprising administering to a hair follicle of the subject a therapeutically effective amount of a Jak1, a Jak2, a Jak3, a Tyk2, a STAT1, a STAT2, a STAT3, a STAT4, a STAT5a, a STAT5b, a STAT6, an OSM, a gp130, an LIFR, and/or an OSM-Rβ inhibitor, and wherein the subject has androgenetic alopecia, telogen effluvium, alopecia areata, tinea capitis, alopecia totalis, hypotrichosis, hereditary hypotrichosis simplex, frontal fibrosing alopecia, cicatricial alopecia, lichen planopilaris, ring alopecia, scarring alopacia, nonscarring alopacia, chemotherapy induced alopecia, or alopecia universalis.

In certain embodiments, the present disclosure is directed to methods of inducing hair growth in a mammalian subject, wherein the method comprising administering to a hair follicle of the subject a therapeutically effective amount of a Jak1, a Jak2, a Jak3, a Tyk2, a STAT1, a STAT2, a STAT3, a STAT4, a STAT5a, a STAT5b, a STAT6, an OSM, a gp130, an LIFR, and/or an OSM-Rβ inhibitor, and wherein the inhibitor is an antisense RNA, an siRNA, an shRNA, a microRNA, or a variant or modification thereof that specifically inhibits expression of the gene that encodes the Jak1, Jak2, Jak3, Tyk2, STAT1, STAT2, STAT3, STAT4, STAT5a, STAT5b, STAT6, OSM, gp130, LIFR, or OSM-Rβ; or a small molecule.

In certain embodiments, the present disclosure is directed to methods of inducing hair growth in a mammalian subject, wherein the method comprising administering to a hair follicle of the subject a therapeutically effective amount of a Jak1, a Jak2, a Jak3, a Tyk2, a STAT1, a STAT2, a STAT3, a STAT4, a STAT5a, a STAT5b, a STAT6, an OSM, a gp130, an LIFR, and/or an OSM-Rβ inhibitor, and wherein the inhibitor is ruxolitinib (INCB 018424).

In certain embodiments, the present disclosure is directed to methods of inducing hair growth in a mammalian subject, wherein the method comprising administering to a hair follicle of the subject a therapeutically effective amount of a Jak1, a Jak2, a Jak3, a Tyk2, a STAT1, a STAT2, a STAT3, a STAT4, a STAT5a, a STAT5b, a STAT6, an OSM, a gp130, an LIFR, and/or an OSM-Rβ inhibitor, and wherein the inhibitor is tofacitinib (CP690550).

In certain embodiments, the present disclosure is directed to methods of inducing hair growth in a mammalian subject, wherein the method comprising administering to a hair follicle of the subject a therapeutically effective amount of a Jak1, a Jak2, a Jak3, a Tyk2, a STAT1, a STAT2, a STAT3, a STAT4, a STAT5a, a STAT5b, a STAT6, an OSM, a gp130, an LIFR, and/or an OSM-Rβ inhibitor, and wherein the inhibitor is ruxolitinib (INCB 018424), tofacitinib (CP690550), AG490, momelotinib (CYT387), partcitinib (SB1518), baricitinib (LY3009104), fedratinib (TG101348), BMS-911543, lestaurtinib (CEP-701), fludarabine, epigallocatechin-3-gallate (EGCG), baricitinib, momelotinib, pacritinib, peficitinib, ABT 494, AT 9283, decernmotinib, filgotinib, gandotinib, INCB 39110, PF 4965842, R348, AZD 1480, BMS 911543, cerdulatinib, INCB 052793, NS 018, C 410, CT 1578, JTE 052, PF 6263276, R 548, TG 02, lumbricus rebellus extract, ARN 4079, AR 13154, UR 67767, CS510, VR588, DNX 04042, or hyperforin or combinations thereof.

In certain embodiments, the present disclosure is directed to methods of inducing hair growth in a mammalian subject, wherein the method comprising administering to a hair follicle of the subject a therapeutically effective amount of a Jak1, a Jak2, a Jak3, a Tyk2, a STAT1, a STAT2, a STAT3, a STAT4, a STAT5a, a STAT5b, a STAT6, an OSM, a gp130, an LIFR, and/or an OSM-Rβ inhibitor, and wherein the inhibitor is an OSM-Rβ antibody.

In certain embodiments, the present disclosure is directed to methods of inducing hair growth in a mammalian subject, wherein the method comprising administering to a hair follicle of the subject a therapeutically effective amount of a Jak1, a Jak2, a Jak3, a Tyk2, a STAT1, a STAT2, a STAT3, a STAT4, a STAT5a, a STAT5b, a STAT6, an OSM, a gp130, an LIFR, and/or an OSM-Rβ inhibitor, wherein the subject is a human.

In certain embodiments, the present disclosure is directed to methods of inducing hair growth in a mammalian subject, the method comprising administering to a hair follicle of the subject a therapeutically effective amount of a Jak1, a Jak2, a Jak3, a Tyk2, a STAT1, a STAT2, a STAT3, a STAT4, a STAT5a, a STAT5b, a STAT6, an OSM, a gp130, an LIFR, and/or an OSM-Rβ inhibitor, wherein the hair is on a scalp or a face, or constitutes an eyebrow or an eyelash of the subject.

In certain embodiments, the present disclosure is directed to methods of inducing hair growth in a mammalian subject, the method comprising administering to a hair follicle of the subject a therapeutically effective amount of a Jak1, a Jak2, a Jak3, a Tyk2, a STAT1, a STAT2, a STAT3, a STAT4, a STAT5a, a STAT5b, a STAT6, an OSM, a gp130, an LIFR, and/or an OSM-Rβ inhibitor, wherein the hair is nasal hair.

In certain embodiments, the present disclosure is directed to methods of inducing hair growth in a mammalian subject, the method comprising administering to a hair follicle of the subject a therapeutically effective amount of a Jak1, a Jak2, a Jak3, a Tyk2, a STAT1, a STAT2, a STAT3, a STAT4, a STAT5a, a STAT5b, a STAT6, an OSM, a gp130, an LIFR, and/or an OSM-Rβ inhibitor, wherein the inhibitor is administered topically.

In certain embodiments, the present disclosure is directed to methods of inducing hair growth in a mammalian subject, the method comprising administering to a hair follicle of the subject a therapeutically effective amount of a Jak1, a Jak2, a Jak3, a Tyk2, a STAT1, a STAT2, a STAT3, a STAT4, a STAT5a, a STAT5b, a STAT6, an OSM, a gp130, an LIFR, and/or an OSM-Rβ inhibitor, wherein the inhibitor is administered orally.

In certain embodiments, the present disclosure is directed to methods of inducing hair growth in a mammalian subject, wherein the method comprising administering to a hair follicle of the subject a therapeutically effective amount of a Jak1, a Jak2, a Jak3, a Tyk2, a STAT1, a STAT2, a STAT3, a STAT4, a STAT5a, a STAT5b, a STAT6, an OSM, a gp130, an LIFR, and/or an OSM-Rβ inhibitor, wherein an expression level of one or more hair growth biomarkers are changed after administering said inhibitors.

In certain embodiments, the present disclosure is directed to methods of inducing hair growth in a mammalian subject, wherein the method comprising administering to a hair follicle of the subject a therapeutically effective amount of a Jak1, a Jak2, a Jak3, a Tyk2, a STAT1, a STAT2, a STAT3, a STAT4, a STAT5a, a STAT5b, a STAT6, an OSM, a gp130, an LIFR, and/or an OSM-Rβ inhibitor, wherein the one or more biomarkers are selected from the group consisting of CD34, Lhx2, NFATc1, Axin2, FoxC1, OSMR, OSM, Jak3, FAS, Irf1, Ifnar1, Nr3c1, Stat5A, Il6st, Ptprc, Ghr, IL10ra, Il2rg, Pdgfra, Spfi1, Socs2, Stat5b, Crp, Il4, Prlr, Insr, IL2ra, Cebpd, Stat3, Jak1, Acvr2a, Sfrp4, Sox5, Cdh2, Fzd5, Wif1, Wnt2, Fzd8, Apc, Sox9, Ilk, Shh, Krt25, Dlx2, Prom1, S100a9, Vegfc, Ptgfr, Pdgfr1, Igfbp4, Gli2, Tyrp1, Syt4, Mlana, Pme1, Dct, Tyr, Sos1, Dbf4, Pax3, PIK3ca, Rps6kb1, Mlph, and Stx17.

In certain embodiments, the present disclosure is directed to methods of promoting hair growth in a mammalian subject, the method comprising administering to a hair follicle of the subject a therapeutically effective amount of a Jak1, a Jak2, a Jak3, a Tyk2, a STAT1, a STAT2, a STAT3, a STAT4, a STAT5a, a STAT5b, a STAT6, an OSM, a gp130, an LIFR, and/or an OSM-Rβ inhibitor.

In certain embodiments, the present disclosure is directed to methods of promoting hair growth in a mammalian subject, the method comprising administering to a hair follicle of the subject a Jak1, a Jak2, a Jak3, a Tyk2, a STAT1, a STAT2, a STAT3, a STAT4, a STAT5a, a STAT5b, a STAT6, an OSM, a gp130, an LIFR, and/or an OSM-Rβ inhibitor, wherein the hair follicle is in a phase other than early telogen phase.

In certain embodiments, the present disclosure is directed to methods of promoting hair growth in a mammalian subject, the method comprising administering to a hair follicle of the subject a Jak1, a Jak2, a Jak3, a Tyk2, a STAT1, a STAT2, a STAT3, a STAT4, a STAT5a, a STAT5b, a STAT6, an OSM, a gp130, an LIFR, and/or an OSM-Rβ inhibitor, wherein the hair follicle is in anagen phase.

In certain embodiments, the present disclosure is directed to methods of promoting inductivity of dermal papilla, wherein the method comprising administering to a dermal papilla 3D sphere derived from a hair follicle of a subject a therapeutically effective amount of a Jak1, a Jak2, a Jak3, a Tyk2, a STAT1, a STAT2, a STAT3, a STAT4, a STAT5a, a STAT5b, a STAT6, an OSM, a gp130, an LIFR, and/or an OSM-Rβ inhibitor, wherein said administration occurs before administering said dermal papilla spheres to a subject.

In certain embodiments, the present disclosure is directed to methods of promoting inductivity of dermal papilla, wherein the method comprising administering to a dermal papilla 3D sphere derived from a hair follicle of a subject a therapeutically effective amount of a Jak1, a Jak2, a Jak3, a Tyk2, a STAT1, a STAT2, a STAT3, a STAT4, a STAT5a, a STAT5b, a STAT6, an OSM, a gp130, an LIFR, and/or an OSM-Rβ inhibitor, wherein said administration occurs before administering said dermal papilla spheres to a subject to treat androgenetic alopecia, telogen effluvium, alopecia areata, tinea capitis, alopecia totalis, hypotrichosis, hereditary hypotrichosis simplex, frontal fibrosing alopecia, cicatricial alopecia, lichen planopilaris, ring alopecia, scarring alopacia, nonscarring alopacia, chemotherapy induced alopecia, or alopecia universalis.

In certain embodiments, the present disclosure further provides methods of assessing the efficacy of a therapy for inducing or promoting hair growth in a mammalian subject, the method comprising: (a) determining a level of one or more hair growth biomarkers in a hair follicle sample obtained from the subject; and (b) determining the level of the one or more hair growth biomarkers in a hair follicle sample obtained from the subject, at one of more time points during the therapy, wherein the therapy is efficacious for inducing or promoting hair growth in the subject when there is a change of the one or more biomarkers in the second or subsequent samples, relative to the first sample.

In certain embodiments, wherein the one or more biomarkers are selected from the group consisting of CD34, Lhx2, NFATc1, Axin2, FoxC1, OSMR, OSM, Jak3, FAS, Irf1, Ifnar1, Nr3c1, Stat5A, Il6st, Ptprc, Ghr, IL10ra, Il2rg, Pdgfra, Spfi1, Socs2, Stat5b, Crp, 114, Prlr, Insr, IL2ra, Cebpd, Stat3, Jak1, Acvr2a, Sfrp4, Sox5, Cdh2, Fzd5, Wif1, Wnt2, Fzd8, Apc, Sox9, Ilk, Shh, Krt25, Dlx2, Prom1, S100a9, Vegfc, Ptgfr, Pdgfr1, Igfbp4, Gli2, Tyrp1, Syt4, Mlana, Pme1, Dct, Tyr, Sos1, Dbf4, Pax3, PIK3ca, Rps6kb1, Mlph, and Stx17.

In certain embodiments, the present disclosure further provides a kit for inducing or promoting hair growth in a mammalian subject, the kit comprising (a) a Jak1, a Jak2, a Jak3, a Tyk2, a STAT1, a STAT2, a STAT3, a STAT4, a STAT5a, a STAT5b, a STAT6, an OSM, a gp130, an LIFR, and/or an OSM-Rβ inhibitor; and (b) a pharmaceutically acceptable carrier.

### 4. BRIEF DESCRIPTION OF FIGURES

FIG. 1A-1E. Inhibition of JAK-STAT signaling restarts anagen in wildtype mice. 1A. 7 weeks old wildtype mice were shaved and treated daily with either a topical application of vehicle control, sonic hedgehog agonist (SAG), 3% ruxolitinib (JAK1/2 inhibitor), tofacitinib (JAK3 inhibitor). Skin was harvested at the indicated time points and stained with H&E. Images of mice were taken at D21 of treatment. 1B. 8.5 week old mice were treated with ruxolitinib, tofacitinib or vehicle control for 5 days, and mice were monitored for the appearance of skin pigmentation, signaling the initiation of anagen. No hair growth (and no pigmentation) was assigned the arbitrary value of 0. Skin darkening was given a value from 0-100%, with higher number indicating darker skin/visible hair growth. 5 mice were used per condition. Nonparametric longitudinal data analysis was performed over days 8-18 post treatment to generate p=7.6×10⁻³⁴ for control vs. ruxolitinib, and P=1.5×10⁻¹⁰ for control vs. Tofacitinib. 1C. Whole skin from mice treated with vehicle control, ruxolitinib and tofacitinib for 4 days was analyzed by microarrays. Expression data were used to identify genes that were differentially expressed between T0 and T5 in each of the three treatment groups. 3 mice were used per condition, biopsied at 2 time points. 1D. Ingenuity Pathway Analysis (IPA) was used to identify the molecular pathways and processes that were overrepresented in the lists of differentially expressed genes. Comparison of the differentially expressed gene lists revealed a subset of genes regulated by both ruxolitinib and tofacitinib. Red=genes upregulated in drug treated T5 vs. T0, Green=genes downregulated in drug treated T5 vs. T0. 1E. 8.5 weeks old mice were treated with ruxolitinib or tofacitinib on one side of their dorsal skin and with vehicle control on the other side. 4 hours after treatment, Edu was injected into each mouse and skin was harvested one hour later. Treatments were performed once, twice and three times and skin was analyzed for presence of Edu+cells

FIG. 2A-2D. The JAK-STAT pathway is dynamically regulated during hair follicle cycling. 2A. 8 weeks old Rag1−/− and Tcr β/δ−/− mice were treated daily with JAK inhibitors. Mice were treated for a week, and images were taken 7 days post cessation of daily treatments. Shown are representative pictures of 3 mice/genotype. 2B. Whole skin was harvested from mice at postnatal day 17 (catagen), day 23 (telogen), day 29 (early anagen) and day 33 (mid anagen). Changes in gene expression were analyzed using a JAK-STAT qPCR array that includes genes involved in the JAK-STAT pathway as well as normalizing controls (Qiagen). 3 mice were used per time point, each hybridized to a separate qPCR plate. Log 2 fold changes in gene expression were utilized to generate GEDI (gene expression dynamics inspector) self-organizing maps (SOMs) in order to visualize the dynamic changes in gene expression over the hair cycle. GEDI clusters transcripts into metagenes based on their similar expression pattern over time, and placed them on a 5×6 grid. Metagenes repressed in experimental samples (D23, D29, D33, respectively) vs. control samples (D17) are in green to blue, while metagenes over represented in experimental samples are red. The upper and lower threshold corresponding to a 2-fold change. Changes larger/smaller than 2× are set to maximum color. 2C. The gene content of the repressed metagenes highlighted in A (boxed pixels) is detailed in the table. Color of boxed pixel corresponds to color of lines in the table 2D. Skin from wildtype mice in anagen (Day 30), catagen (Day 42) and telogen (D50) was harvested, fixed and stained with anti-phospho STAT3 and anti-phospho STAT5, as well as with Krt15 (a bulge marker) and P-cadherin (hair germ marker). Phospho STAT 3 is expressed in extra-follicular cells during anagen, as well as in DP cells (white arrows). In catagen, phospho-STAT3 is expressed in the DP (red arrow) and the hair germ (orange arrow). In early telogen, phospho STAT 3 is present in the hair germ cells that are closest to the DP (green arrow). Phospho STAT 5 is strongly expressed in the DP throughout the hair cycle, with expression peaking during catagen (yellow arrows). Phospho-STAT 5 can also be detected in the bulge of catagen follicles (magenta arrow). Images were taken on Zeiss confocal microscope, 40× magnification

FIG. 3A-3E. Inhibition of JAK-STAT promotes hair growth in human tissue. 3A. Human scalp skin was grafted onto SCID mice and treated topically with vehicle control, ruxolitinib or tofacitinib for 4 weeks. Pictures of the grafts were taken every 3-5 days. To quantify differences between vehicle- and drug-treated sides, ImageJ was used to measure the intensity of pigmentation across the graft. The vertical line on each histogram corresponds to the boundary between vehicle and drug treatment. 3B. Individual HF were dissected from adult human scalp tissue, and placed in culture in presence of vehicle control, ruxolitinib and tofacitinib. Pictures of follicles were taken every 2 days for 2 weeks, and the length of each follicle was measured over time. Treatment with tofacitinib and ruxolitinib significantly increased the growth rate of anagen (P=0.017, and P=0.025, respectively) and therefore the overall length of HF. Experiment pictured was performed with follicles derived from a single donor with 3-4 follicles/condition. P-values were obtained using non-parametric longitudinal data analysis. 3C. DP spheres were grown in hanging drops in the presence of vehicle control, ruxolitinib or tofacitinib, and then combined with mouse neonatal keratinocytes and injected in vivo. 3D. Graph represents 3 separate experiments with spheres generated from 3 individual donors. One injection of human DP spheres and mouse keratinocytes slurry was performed per DP donor/condition resulting in cysts containing induced follicles. 3E. Graphs of numbers of follicles induced after cysts were dissociated and hair fibers were manually counted on a microscope. Difference between control and tofacitinib treatments is statistically significant (P=0.00013). P-values were calculated using a linear mixed effect analysis treating donor as a random effect.

FIG. 4A-4D. Tofacitinib enhances the inductive molecular signature. 4A. DP spheres treated with vehicle control, ruxolitnib or tofacitinib were molecularly profiled using microarrays. Cells from 3 individual donors were used per condition. Log 2 fold changes in gene expression were utilized to generate GEDI plots visualizing the dynamic changes in gene expression over drug treatments (Z axis and color scale). Clusters of transcripts, grouped into metagenes, were placed on a 18×19 grid (X,Y). 3 comparisons are shown: ruxolitinib vs. control, tofacitinib vs. control and ruxolitinib vs. tofacitinib. 4 regions of interest are highlighted on the plots, reflecting genes repressed by tofacitinib treatment (regions 1 & 2) and genes upregulated by tofacitinib treatment (regions 3 & 4). 4B. Selected genes from region 1-4 are presented in the table. 4C. Gene set enrichment analysis revealed that groups of genes previously shown to contribute to DP inductivity (genes within T2 and T4 as identified by Higgins et al. (35) are significantly enriched by tofacitinib treatment (p=1.3×10-5). The grey dotted line shows what a normal distribution (with no enrichment) looks like in this analysis. X-axis describes the rank of all genes passing call in the array ranked from most overexpressed to most underexpressed comparing tofacitinib-treated to untreated cells. The y-axis plots the enrichment score (ES) of the combined gene sets T2 and T4 at a given rank. The normalized enrichment score (NES) reflects the Z-score probability of obtaining the observed ES distribution in randomized data, with the associated p-value reported. Individual gene ranks for genes in T2 and T4 are represented as black hashes in the barcodes beneath the plot. 4D. Summary of the results obtained in FIGS. 3 and 4: While intact DP is fully inductive, the potential to induce hair follicle growth is lost when dermal papillae are cultured in vitro. The inventors previously showed that growing DP cells in spheroid cultures restores some of the inductive potential, by regulating a subset of genes in the molecular signature associated with intact DP (territories 1 and 3 (T1 and T3)) (35). Treating DP spheres with tofacitinib increased the inductivity of cultured DP by targeting some of the pathways not previously altered by the 3-dimensional culture condition (T2 and T4). Blue=gene expression within territories corresponding on non-Inductive state, Red=gene expression within territories corresponding to inductive state. Although tofacitinib restores genes within T2 and T4, restoration is incomplete, compared to fully intact DP (territories in bright red).

FIG. 5A-5E: JAK inhibition mediated hair growth recapitulates endogenous hair growth and is dependent on the duration of telogen. 5A. Wildtype mice in telogen (8.5 weeks) were treated with vehicle control, 2% tofacitinib or 2% ruxolitinib for 4 days. Skin was harvested, sectioned and stained with Ki67 (proliferation marker) and P-cadherin (hair germ marker). Images are representative of 3 mice/treatment. 5B. Wildtype mice at 7 weeks of age were treated with vehicle, tofacitinib or ruxolitinib, and skin was harvested at the indicated time points. The numbers and thickness of follicles at these time points were quantified. As a control, hair growth post depilation, a process that proceeds in a highly stereotyped manner, was utilized (8). 5C. Two groups of 7 week old or 8.5 week old mice were shaved and treated daily with ruxolitinib, tofacitinib or vehicle control. Mice were treated for a week, and images were taken 7 days post cessation of daily treatment. Skin was harvested and stained with H&E. To ensure that all mice were still in refractory telogen, it is shown that hair growth following plucking follows a similar kinetics in both age groups. Images of mice and H&E staining are representative of 2 experiments, with 3 mice per group. 5D. 7 week old mice (post natal day 49) were treated with ruxolitinib, tofacitinib or vehicle control for 24 days, with biopsies taken at the indicated time points. As shown, by 18 days of treatment with ruxolitinib and 21 days of treatment with tofacitinib, treated follicle re-initiate anagen. 5E. 8.5 week old mice were treated daily with control (C left side) or ruxolitinib (R right side). Pictures were taken daily, starting 5 days post treatment, and skin was scored for level of skin darkening. Shown are representative pictures of treated from mice (4 mice/treatment group) on day 8, 12,16 post treatment.

FIG. 6A-6C: Molecular pathways enriched in genes differentially regulated by each JAK-STAT inhibitor. 6A. Whole skin samples treated with ruxolitinib and tofacitinib for 0 (T0) or 4 days (T5) were analyzed by qPCR for selected genes identified by the microarray analysis. Results represent 3 separate experiments, p values>0.05, as determined by paired t-test. 6B. Microarray expression data were used to identify genes that were differentially expressed between T0 and T5 in DMSO, ruxolitinib and tofacitinib treated groups. Ingenuity Pathway Analysis (IPA) was used to identify the genes that were differentially regulated in all treatments, a subset of treatments, or exclusively by each condition. Next, IPA was utilized to explore the molecular pathways and processes that were overrepresented in the lists of differentially expressed genes. Shown are pathways enriched by ruxolitinib treatment. 6C. pathways enriched by tofacitinib treatment. Red=genes upregulated in the drug treated samples, Green=genes downregulated in drug treated samples.

FIG. 7A-7C: Effects of JAK inhibition are independent of T cells and instead represent cell intrinsic properties. 7A. To establish whether there were any hair cycling abnormalities in lymphocyte deficient mice, the inventors harvested and analyzed skin from Rag1−/− mice, deficient in B and T cells and Tcr β/δ−/− mice, deficient in migrating and resident T cells, as well as heterozygous controls at post natal day 30 (anagen), Day 42 (catagen) and D50 (early telogen). Representative H&E images of 3 mice/genotype are shown. 7B. Changes in gene expression over time points, representing the transition from catagen to the following anagen, were plotted for a selected number of genes. 7C. Lower magnification images of phospho-STAT 3 and phospho-STAT 5. Images were taken on Zeiss confocal microscope, 20× magnification.

FIG. 8A-8E: Inhibition of JAK-STAT promotes human hair follicle growth. 8A. Human scalp skin was grafted onto SCID mice and treated topically with vehicle control, ruxolitinib or tofacitinib for 4 weeks. Quantification of the darkness of skin over time is shown. 8B. A second example of treatment of human embryonic scalp skin with tofacitinib. In this case, the graft was not large enough to allow for multiple treatments, therefore treatments were performed on two separate grafts from the same individual. 8C. The organ culture experiment was repeated with follicles from two individuals. Treatment with tofacitinib and ruxolitinib increased the growth rate of anagen, repeating the trend observed before, but results did not reach statistical significance. 8D. human HF in anagen were stained with phospho STAT 3, Phospho STAT 5 or isotype control. Images were taken on the Zeiss Confocal Microscope, 20× magnification. 8E. T2 was also enriched by tofacitinib treatment, however the statistical overlap was only marginally significant (P=0.03).

FIG. 9A-9E: OSM and OSM-Rβ co-localize with activated STAT3 and STAT5 in the bulge during telogen. A. Phosphorylated STAT3 and STAT5 proteins are present in the HFSC compartment during early and mid telogen (P46, P60), and decrease during late telogen (P80). OSM immunofluorescence is co-localized to the HFSC during this time. B. OSM receptor (OSM-12(3) is also co-localize to the HFSC bulge compartment with immunohistochemistry. All images were taken at ×20 magnification. C. In vitro cultured keratinocytes, OSM activates STAT1, STAT3 and STAT5 transcription factors within 10 minutes of OSM treatment, and this is efficiently abrogated by Tofacitinib (TOFA). D. In vivo whole skin treated with TOFA also demonstrates effective inhibition of STAT3 and STAT5 activation. E. Neutralizing antibodies to OSM-Rβ injected subcutaneously daily into P60 telogen skin induce a local anagen. PBS control and neutralizing antibodies to IL-6 and its receptor show no effect. F. gp130 is also enriched in the bulge.

FIG. 10A-10G: OSM has growth-inhibitory properties on mouse keratinocytes, and acts on the HF stem cell compartment in the bulge. A. Colony forming assay with mouse keratinocytes harvested at telogen show that OSM have a growth inhibitory effect on keratinocytes, which is reversed partially with the addition of Tofacitinib. B. Tofacitinib alone increase the number of keratinocyte colonies. C. Skin harvested in the second telogen at 7.5 and 8.5 weeks (P56 and P60) were dissociated and the epidermis and dermis were analyzed by qRT-PCR. D. OSM-Rβ is preferentially expressed by the epidermis, while OSM is produced in the dermis. OSM production also increases between P53 and P60. E. In the epidermis, OSM-Rβ is enriched in the bulge compartment. F. Cell sorting strategy for mouse epidermis to isolate CD34+ ITGA6+ HFSCs. G. In cultured mouse keratinocytes, treatment with recombinant mouse OSM at 20 ng/ml upregulates the expression of OSM-Rβ.

FIG. 11A-11F: JAK inhibition suppresses OSM-Rβ expression in mouse skin, potentially leading to loss of quiescence. A. Pre-existing microarray data from JAK-inhibitor experiments were re-analyzed and JAK inhibition produced a consistent downregulation of OSM-Rβ whole mouse skin treated during telogen. B. Downregulation of OSM-Rβ confirmed with qRT-PCR in vivo, where whole skin biopsies were collected daily over a period of 5 days of Tofacitinib or Ruxolitinib treatment. C. Downregulation of OSM-Rβ transcription in vitro in cultured mouse keratinocytes. D. Sorted keratinocytes from telogen mouse skin treated with Ruxolitinib also exhibits a downregulation of OSM-Rβ after 3 days. E. In vivo treatment with Ruxolitinib, a JAK2 inhibitor, leads to early STAT3/5 inhibition, then an activation of Akt and ERK pathways, leading to a downregulation of OSM-Rβ. F. A schematic shwong that OSM produced in the dermal compartment, possibly the dermal papilla, engages OSM-Rβ on the HFSCs in the bulge to maintain quiescence during telogen by suppressing growth and differentiation, at the same time maintaining OSM-Rβ expression.

Table 1: List of antibodies and dilutions used in this study

Table 2: List of qPCR primers used in this study

Table 3: List of genes within territories T2 and T4 (as adapted from Higgins et al., PNAS 2013)

### 5. DETAILED DESCRIPTION

For clarity and not by way of limitation the detailed description of the invention is divided into the following subsections:

5.1 Definitions

5.2 JAK-STAT Pathway Genes

5.3 Methods of Treatment

5.4 Pharmaceutical Compositions and Administration

5.5 Methods of Monitoring Efficacy of Treatment

5.6 Kits.

Recently, the inventors demonstrated that pharmacological inhibition of the JAK-STAT pathway promotes rapid hair regrowth in alopecia areata (AA) in both mice and humans (1) (WO2013149194 A1 to Christiano, et al., incorporated herein). Unexpectedly, during the course of the studies on mice with AA, it was observed that topical treatment with JAK-STAT inhibitors resulted in an unusually robust hair growth, suggesting a localized effect on initiation of the hair cycle. As disclosed herein, pharmacological inhibition of JAK-STAT signaling initiates the hair cycle in normal mice and promotes hair growth in humans.

In light of the foregoing, the presently disclosed subject matter relates, in certain embodiments, to compositions and methods for the inhibition of the JAK-STAT pathway to induce hair growth. In certain embodiments, the presently disclosed subject matter relates to topical treatments with small molecule inhibitors of the JAK-STAT pathway to induce hair growth.

### 5.1 Definitions

According to the present disclosure, a “subject” or a “patient” is a human or non-human animal. Although the animal subject is preferably a human, the compounds and compositions of the invention have application in veterinary medicine as well, e.g., for the treatment of domesticated species such as canine, feline, murine, and various other pets; farm animal species such as bovine, equine, ovine, caprine, porcine, etc.; and wild animals, e.g., in the wild or in a zoological garden, such as non-human primates.

“Pharmaceutical composition” and “pharmaceutical formulation,” as used herein, refer to a composition which is in such form as to permit the biological activity of an active ingredient contained therein to be effective, and which contains no additional components which are unacceptably toxic to a patient to which the formulation would be administered.

“Pharmaceutically acceptable,” as used herein, e.g., with respect to a “pharmaceutically acceptable carrier,” refers to the property of being nontoxic to a subject. A pharmaceutically acceptable ingredient in a pharmaceutical formulation can be an ingredient other than an active ingredient which is nontoxic. A pharmaceutically acceptable carrier can include a buffer, excipient, stabilizer, and/or preservative.

As used herein, a “Jak1 inhibitor” refers to a compound that interacts with a Jak1 gene or a Jak1 protein or polypeptide and inhibits its activity and/or its expression. The compound can decrease the activity or expression of a protein encoded by Jak1.

As used herein, a “Jak2 inhibitor” refers to a compound that interacts with a Jak2 gene or a Jak2 protein or polypeptide and inhibits its activity and/or its expression. The compound can decrease the activity or expression of a protein encoded by Jak2.

As used herein, a “Jak3 inhibitor” can be a compound that interacts with a Jak 3 gene, or a Jak 3 protein or polypeptide, and inhibits its activity and/or its expression. The compound can decrease the activity or expression of a protein encoded by Jak3. In one embodiment, a Jak3 inhibitor can be a Jak3 modulating compound.

As used herein, a “Tyk2 inhibitor” can be a compound that interacts with a Tyk2 gene, or a Tyk2 protein or polypeptide, and inhibits its activity and/or its expression. The compound can decrease the activity or expression of a protein encoded by Tyk2. In one embodiment, a Tyk2 inhibitor can be a Tyk2 modulating compound

As used herein, a “STAT1 inhibitor” refers to a compound that interacts with a STAT1 gene or a STAT1 protein or polypeptide and inhibits its activity and/or its expression. The compound can decrease the activity or expression of a protein encoded by STAT1.

As used herein, a “STAT2 inhibitor” refers to a compound that interacts with a STAT2 gene or a STAT2 protein or polypeptide and inhibits its activity and/or its expression. The compound can decrease the activity or expression of a protein encoded by STAT2.

As used herein, a “STAT3 inhibitor” refers to a compound that interacts with a STAT3 gene or a STAT3 protein or polypeptide and inhibits its activity and/or its expression. The compound can decrease the activity or expression of a protein encoded by STAT3.

As used herein, a “STAT4 inhibitor” refers to a compound that interacts with a STAT4 gene or a STAT4 protein or polypeptide and inhibits its activity and/or its expression. The compound can decrease the activity or expression of a protein encoded by STAT4.

As used herein, a “STAT5a inhibitor” refers to a compound that interacts with a STAT5a gene or a STAT5a protein or polypeptide and inhibits its activity and/or its expression. The compound can decrease the activity or expression of a protein encoded by STAT5a.

As used herein, a “STAT5b inhibitor” refers to a compound that interacts with a STAT5b gene or a STAT5b protein or polypeptide and inhibits its activity and/or its expression. The compound can decrease the activity or expression of a protein encoded by STAT5b.

As used herein, a “STAT6 inhibitor” refers to a compound that interacts with a STAT6 gene or a STAT6 protein or polypeptide and inhibits its activity and/or its expression. The compound can decrease the activity or expression of a protein encoded by STAT6.

As used herein, an “OSM inhibitor” refers to a compound that interacts with an OMS gene or an OMS protein or polypeptide and inhibits its activity and/or its expression. The compound can decrease the activity or expression of a protein encoded by OSM.

As used herein, a “gp130 inhibitor” refers to a compound that interacts with a gp130 gene or a gp130 protein or polypeptide and inhibits its activity and/or its expression. The compound can decrease the activity or expression of a protein encoded by gp130.

As used herein, an “LIFR inhibitor” refers to a compound that interacts with an LIFR gene or an LIFR protein or polypeptide and inhibits its activity and/or its expression. The compound can decrease the activity or expression of a protein encoded by LIFR.

As used herein, an “OSM-Rβ inhibitor” refers to a compound that interacts with an OSM-Rβ gene or an OSM-Rβ protein or polypeptide and inhibits its activity and/or its expression. The compound can decrease the activity or expression of a protein encoded by OSM-Rβ.

As used herein, a “Jak/STAT inhibitor” refers to a compound that interacts with a Jak1/Jak2/Jak3/Tyk2/STAT1/STAT2/STAT3/STAT4/STAT5a/STAT5b/STAT6/OS M/gp130/LIFR/OSM-Rβ gene or a Jak1/Jak2/Jak3/Tyk2/STAT1/STAT2/STAT3/STAT4/STAT5a /STAT5b/STAT6/OSM/gp130/LIFR/OSM-Rβ protein or polypeptide and inhibits its activity and/or its expression. The compound can decrease the activity or expression of a protein encoded by Jak1/Jak2/Jak3/Tyk2/STAT1/STAT2/STAT3/STAT4/STAT5a/STAT5b/STAT6/OS M/gp130/LIFR/OSM-Rβ.

An inhibitor of the present disclosure can be a protein, such as an antibody (monoclonal, polyclonal, humanized, chimeric, or fully human), or a binding fragment thereof, directed against a polypeptide encoded by the corresponding sequence disclosed herein. An antibody fragment can be a form of an antibody other than the full-length form and includes portions or components that exist within full-length antibodies, in addition to antibody fragments that have been engineered, Antibody fragments can include, but are not limited to, single chain Fv (scFv), diabodies, Fv, and (Fab′)₂, triabodies, Fc, Fab, CDR1, CDR2, CDR3, combinations of CDR's, variable regions, tetrabodies, bifunctional hybrid antibodies, framework regions, constant regions, and the like (see, Maynard et al., (2000) Ann. Rev. Biomed. Eng. 2:339-76; Hudson (1998) Curr. Opin. Biotechnol. 9:395-402). Antibodies can be obtained commercially, custom generated, or synthesized against an antigen of interest according to methods established in the art (Janeway et al., (2001) Immunobiology, 5th ed., Garland Publishing).

An inhibitor of the present disclosure can be a small molecule that binds to a protein and disrupts its function. Small molecules are a diverse group of synthetic and natural substances generally having low molecular weights. They can be isolated from natural sources (for example, plants, fungi, microbes and the like), are obtained commercially and/or available as libraries or collections, or synthesized. Candidate small molecules that modulate a protein can be identified via in silico screening or high-through-put (HTP) screening of combinatorial libraries. Most conventional pharmaceuticals, such as aspirin, penicillin, and many chemotherapeutics, are small molecules, can be obtained commercially, can be chemically synthesized, or can be obtained from random or combinatorial libraries (Werner et al., (2006) Brief Funct. Genomic Proteomic 5(1):32-6). In some embodiments, the agent is a small molecule that binds, interacts, or associates with a target protein or RNA. Such a small molecule can be an organic molecule that, when the target is an intracellular target, is capable of penetrating the lipid bilayer of a cell to interact with the target. Small molecules include, but are not limited to, toxins, chelating agents, metals, and metalloid compounds. Small molecules can be attached or conjugated to a targeting agent so as to specifically guide the small molecule to a particular cell.

As used herein, “therapeutically effective amount” refers to the amount of the inhibitors of the present disclosure contained in the composition administered is of sufficient quantity to achieve the intended purpose, such as, in this case, to induce or promote hair growth in the subject. For the purpose of the present disclosure, methods of measuring hair growth is well known in the art, and need not be repeated herein. In the context of administering a inhibitor to induce hair growth, an effective amount of a composition is an amount sufficient to cause the hair follicle to re-enter anagen phase. In the context of administering a inhibitor to promote hair growth in anagen phase, an effective amount of an inhibitor is an amount sufficient to increase the rate of hair growth. For example, the increase can be a 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, 600%, 700%, 800%, 900%, 1000%, 5000%, 10000% or more increase in the rate of hair growth. An therapeutically effective amount for each administration can be any amount between 1 ng to 1 ug, 1 ug to 1 mg, 2 mg, 3 mg, 4 mg, 5 mg, 6 mg, 7 mg, 8 mg, 9 mg, 10 mg, 20 mg, 30 mg, 40 mg, 50 mg, 100 mg, 200 mg, 300 mg, 400 mg, 500 mg, 1 g or more, or any intermediate amount thereof. An therapeutically effective amount can be administered in one or more administrations. An therapeutically effective amount of the inhibitors can be administered topically, orally or intravenously. When used in an admixture with a pharmaceutically acceptable diluent, carrier or excipient, an effective amount of an inhibitor can be an amount of 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10% or more by weight, or any intermediate amount thereof.

As used herein, “anagen phase” refers to the active growth phase of hair follicles. Typically, in anagen phase, the cells at the root of the hair is dividing rapidly, adding to the hair shaft. At the end of the anagen phase, certain biological signal causes the follicle to enter the catagen phase.

As used herein, “catagen phase” refers to a transition stage that occurs at the end of the anagen phase. It signals the end of the active growth of a hair.

As used herein, “telogen phase” refers to the resting phase of the hair follicle. During the telogen phase the follicle remains dormant. Under certain biological signal, the follicle will re-enter anagen phase and begin to grow again. Early telogen phase refers to the 5%-40% of the time span at the beginning of the telogen phase. Late telogen phase refers to the 5%-40% of the time span at the end of the telogen phase. Mid-telogen phase refers to the time period between the early telogen phase and the late telogen phase.

As used herein, “androgenetic alopecia”, also known as male-pattern hair loss, is hair loss that occurs due to an underlying susceptibility of hair follicles to shrinkage due to the influence of androgenic hormones.

As used herein, “telogen effluvium” is a scalp disorder characterized by the thinning or shedding of hair resulting from the early entry of hair in the telogen phase (the resting phase of the hair follicle)

As used herein, “alopecia areata”, also known as spot baldness, is an autoimmune disease in which hair is lost from some or all areas of the body due to the body's failure to recognize its own cells and subsequent destruction of its own tissue.

As used herein, “tinea capitis”, is a cutaneous fungal infection (dermatophytosis) of the scalp. The disease is primarily caused by dermatophytes in the Trichophyton and Microsporum genera that invade the hair shaft. The clinical presentation is typically single or multiple patches of hair loss, sometimes with a ‘black dot’ pattern (often with broken-off hairs), that may be accompanied by inflammation, scaling, pustules, and itching.

As used herein, “alopecia totalis” refers to the loss of all head hair.

As used herein, “hypotrichosis” refers to a condition of abnormal hair patterns—predominantly loss or reduction.

As used herein, “hereditary hypotrichosis simplex” refers to a genetic disorder, characterized by sparse or absent scalp hair without structural defects, in the absence of other ectodermal or systemic abnormalities.

As used herein, “frontal fibrosing alopecia” refers to a form of scarring hair loss affecting the hair margin on the front of the scalp.

As used herein, “cicatricial alopecia” also called scarring alopecia, refers to a group of rare disorders that destroy hair follicles. The follicles are replaced with scar tissue, causing permanent hair loss.

As used herein, “lichen planopilaris” is a type of scarring hair loss that occurs when a relatively common skin disease, known as lichen planus, affects areas of the skin with hair. Lichen planopilaris destroys the hair follicle replacing it with scarring.

As used herein, “ring alopecia” is a ring or band of alopecia encircling or partially encircling the head; it may extend along the posterior occipital area, around the temporal portion of the scalp above the ears or onto the forehead.

As used herein, “chemotherapy induced alopecia” is a type of hair loss that occurs after chemotherapy for treatment of cancer or non-cancer diseases such as lupus and rheumatoid arthritis.

As used herein, “Alopecia universalis” refers to a condition characterized by the complete loss of hair on the scalp and body. It is an advanced form of alopecia areata, a condition that causes round patches of hair loss.

### 5.2 JAK-STAT Pathway Genes

The JAK-STAT signalling pathway transmits biological signals from extracellular environment to the nucleus and causes DNA transcription and expression of genes involved in differentiation, apoptosis, immunity, proliferation, and oncogenesis. The three main components of the pathway are a cell surface receptor, a Janus kinase (JAK) and a Signal Transducer and Activator of Transcription (STAT) protein.

JAK is a family of intracellular, nonreceptor tyrosine kinases. STAT is a family of intracellular transcription factors. The binding of ligands such as interferon, interleukin, and/or growth factors to cell surface receptors activate associated JAKs, which phosphorylate tyrosine residues on the receptor, creating binding sites for SH2 domains. STATs, which contain SH2 domain, are then recruited to the receptor whereby they are also tyrosine-phosphorylated by JAKs. The activated STATs form heterodimers or homodimers and translocate to the cell nucleus to induce transcription of target genes. STATs may also be tyrosine-phosphorylated directly by receptor tyrosine kinases (e.g., epidermal growth factor receptor) and/or non-receptor tyrosine kinases (e.g., c-src).

The JAK family genes comprises Janus kinase 1 (JAK1, GenBank ID: 3716), Janus kinase 2 (JAK2, GenBank ID: 3717), Janus kinase 3 (JAK3, GenBank ID: 3718), and Tyrosine kinase 2 (TYK2, GenBank ID: 7297).

The STAT family genes comprises signal transducer and activator of transcription 1 (STAT1, GenBank ID: 6772), signal transducer and activator of transcription 2 (STAT2, GenBank ID: 6773), signal transducer and activator of transcription 3 (STAT3, GenBank ID: 6774), signal transducer and activator of transcription 4 (STAT4, GenBank ID: 6775), signal transducer and activator of transcription 5A (STAT5A, GenBank ID: 6776), signal transducer and activator of transcription 5B (STAT5B, GenBank ID: 6777), and signal transducer and activator of transcription 6 (STAT6, GenBank ID: 6778).

Oncostatin M (OSM, GenBank ID: 5008) is a gene encoding a member of the leukemia inhibitory factor/oncostatin-M (LIF/OSM) family of proteins. The encoded preproprotein is proteolytically processed to generate the mature protein. This protein is a secreted cytokine and growth regulator that inhibits the proliferation of a number of tumor cell lines. This protein also regulates the production of other cytokines, including interleukin 6, granulocyte-colony stimulating factor and granulocyte-macrophage colony stimulating factor in endothelial cells. OSM mediates its bioactivities through two different heterodimer receptors. The gp130 receptor is the common component, which dimerizes with either leukemia inhibitory factor receptor (LIFR) or with OSM receptor β (OSM-Rβ) to generate, respectively, type I and type II OSM receptors. Both type I and type II OSM receptors activated JAK-STAT signal pathway.

Glycoprotein 130 (gp130, GenBank ID: 3572, also known as interleukin 6 signal transducer, IL6ST, IL6-beta or CD130) is a transmembrane signal transducer protein shared by many cytokines, including interleukin 6 (IL6), ciliary neurotrophic factor (CNTF), leukemia inhibitory factor (LIF), and oncostatin M (OSM). This protein functions as a part of the cytokine receptor complex. The activation of this protein is dependent upon the binding of cytokines to their receptors.

OSM receptor β (OSM-Rβ, GenBank ID: 9180, also known as the oncostatin M receptor, or OSMR) is a gene encoding a member of the type I cytokine receptor family. The encoded protein heterodimerizes with gp130 to form the type II oncostatin M receptor and with interleukin 31 receptor A to form the interleukin 31 receptor, and thus transduces oncostatin M and interleukin 31 induced signaling events.

Leukemia inhibitory factor receptor (LIFR, GenBank ID: 3977, also known as leukemia inhibitory factor receptor alpha) is a gene encoding a protein that belongs to the type I cytokine receptor family. This protein combines with a high-affinity converter subunit, gp130, to form a receptor complex that mediates the action of the leukemia inhibitory factor, a polyfunctional cytokine that is involved in cellular differentiation, proliferation and survival in the adult and the embryo.

### 5.3 Methods of Treatment

### Method of Inducing or Promoting Hair Growth

The present disclosure relates to the use of a therapeutically effective amount of one or more JAK/STAT protein (e.g., Jak1, Jak2, Jak3, Tyk2, STAT1, STAT2, STAT3, STAT4, STAT5a, STAT5b, STAT6, OSM, gp130, LIFR, or OSM-Rβ) inhibitors (e.g., ruxolitinib (INCB 018424), tofacitinib (CP690550), AG490, CYT387, SB1518, LY3009104, TG101348, BMS-911543, CEP-701, fludarabine, epigallocatechin-3-gallate (EGCG), baricitinib, momelotinib, pacritinib, peficitinib, ABT 494, AT 9283, decernmotinib, filgotinib, gandotinib, INCB 39110, lestaurtinib, PF 4965842, R348, AZD 1480, BMS 911543, cerdulatinib, INCB 052793, NS 018, C 410, CT 1578, JTE 052, PF 6263276, R 548, TG 02, lumbricus rebellus extract, ARN 4079, AR 13154, UR 67767, CS510, VR588, DNX 04042, or hyperforin), to induce or promote hair growth. Non-limiting contexts where such induction or promotion of hair growth would desirable include, but are not limited to, those contexts where a subject has androgenetic alopecia, telogen effluvium, alopecia areata, tinea capitis, alopecia totalis, hypotrichosis, hereditary hypotrichosis simplex, frontal fibrosing alopecia, cicatricial alopecia, lichen planopilaris, ring alopecia, scarring alopacia, nonscarring alopacia, chemotherapy induced alopecia, or alopecia universalis. In certain embodiments, the inhibitor administered to a subject's hair follicle when the hair follicle is in mid-telogen phase or late-telogen phase. In certain embodiments, the inhibitor is administered topically or orally.

In certain embodiments, the present disclosure is directed to methods of inducing hair growth in a subject in need thereof, the method comprising administering to the subject a therapeutically effective amount of a Jak/STAT inhibitor. In certain embodiments, the Jak/STAT inhibitor is an antibody that specifically binds to a Jak/STAT protein or a fragment thereof; an antisense RNA, antisense DNA, an siRNA, an shRNA, a microRNA, or a variant or modification thereof that decreases expression of the gene that encodes the Jak/STAT protein; an antisense RNA, antisense DNA, an siRNA, an shRNA, a microRNA, or a variant or modification thereof that decreases expression of the Jak/STAT protein; a small molecule; or a combination thereof. In certain embodiments, the inhibitor is ruxolitinib (INCB 018424). In certain embodiments, the inhibitor is tofacitinib (CP690550). In a certain embodiments, the inhibitor is ruxolitinib (INCB 018424), tofacitinib (CP690550), AG490, momelotinib (CYT387), partcitinib (SB1518), baricitinib (LY3009104), fedratinib (TG101348), BMS-911543, lestaurtinib (CEP-701), fludarabine, epigallocatechin-3-gallate (EGCG), baricitinib, momelotinib, pacritinib, peficitinib, ABT 494, AT 9283, decernmotinib, filgotinib, gandotinib, INCB 39110, PF 4965842, R348, AZD 1480, BMS 911543, cerdulatinib, INCB 052793, NS 018, C 410, CT 1578, JTE 052, PF 6263276, R 548, TG 02, lumbricus rebellus extract, ARN 4079, AR 13154, UR 67767, CS510, VR588, DNX 04042, or hyperforin or combinations thereof. In certain embodiments, the inhibitor is an antibody to OSM, gp130, LIFR, OSM-Rβ or any combinations thereof.

Methods for delivering the small molecule, the antisense RNA, antisense DNA, siRNA, shRNA, microRNA, or any variant or modification thereof can vary depending on the need. In certain embodiments, the components of a selected agent are delivered as DNA constructs in one or more plasmids. In certain embodiments, the components are delivered via viral vectors. Common delivery methods include but are not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

In certain embodiments, gene expression level of one or more hair growth biomarkers. As used herein, hair growth biomarkers comprises genes listed in Table 3, any genes in Wnt pathway, Shh pathway, hair development pathway and melanogenesis pathway, and the group comprising CD34, Lhx2, NFATc1, Axin2, FoxC1, OSMR, OSM, Jak3, FAS, Irf1, Ifnar1, Nr3c1, Stat5A, Il6st, Ptprc, Ghr, IL10ra, Il2rg, Pdgfra, Spfi1, Socs2, Stat5b, Crp, Il4, Prlr, Insr, IL2ra, Cebpd, Stat3, Jak1, Acvr2a, Sfrp4, Sox5, Cdh2, Fzd5, Wif1, Wnt2, Fzd8, Apc, Sox9, Ilk, Shh, Krt25, Dlx2, Prom1, S100a9, Vegfc, Ptgfr, Pdgfr1, Igfbp4, Gli2, Tyrp1, Syt4, Mlana, Pme1, Dct, Tyr, Sos1, Dbf4, Pax3, PIK3ca, Rps6kb1, Mlph, and Stx17. In certain embodiments, the expression level of the one or more biomarkers are selected from the group consisting of genes listed in Table 3.

In certain embodiments, the expression level of the one or more biomarkers are selected from the group consisting of CD34, Lhx2, NFATc1, Axin2, FoxC1, OSMR, OSM, Jak3, FAS, Irf1, Ifnar1, Nr3c1, Stat5A, Il6st, Ptprc, Ghr, IL10ra, Il2rg, Pdgfra, Spfi1, Socs2, Stat5b, Crp, Il4, Prlr, Insr, IL2ra, Cebpd, Stat3, Jak1, Acvr2a, Sfrp4, Sox5, Cdh2, Fzd5, Wif1, Wnt2, Fzd8, Apc, Sox9, Ilk, Shh, Krt25, Dlx2, Prom1, S100a9, Vegfc, Ptgfr, Pdgfr1, Igfbp4, Gli2, Tyrp1, Syt4, Mlana, Pme1, Dct, Tyr, Sos1, Dbf4, Pax3, PIK3ca, Rps6kb1, Mlph, and Stx17.

In certain embodiments, the one or more biomarkers are selected from genes in Wnt pathway, Shh pathway, hair development pathway, melanogenesis pathway, or any combination thereof, is changed after administering said inhibitors.

The Wnt and Shh signaling pathways are cell signal pathways that pass signals into a cell through cell surface receptors. Genes involved in Wnt pathways include, but is not limited to, Aes (TLE/Groucho), Apc, Axin1, Bcl9, Csnk1a1, Csnk1d, Csnk1g1, Csnk2a1, Ctbp1, Ctbp2, Ctnnb1, Ctnnbip1 (Icat), Cxxc4, Dixdc1, Dkk1, Dv11, Dv12, Ep300, Frat1, Fzd1, Fzd2, Fzd3, Fzd4, Fzd5, Fzd6, Fzd7, Fzd8, Gsk3a, Gsk3b, Lef1, Lrp5, Lrp6, Nkd1, Porcn, Ppp2ca, Ppp2r1a, Pygo1, Senp2, Sfrp1, Sfrp4, Sox17, Tcf7, Tcf711, Wif1, Wnt1, Wnt10a, Wnt16, Wnt2, Wnt2b, Wnt3, Wnt3a, Wnt4, Wnt6, Wnt7a, Wnt7b, and Wnt8a. Genes involved in Wnt pathways include, but is not limited to: Dhh, Hhat, Hhip, Ihh, Shh, Siah1, C18orf8, C6orf138, Npc1, Npc111, Ptch1, Ptch2, Ptchd1, Ptchd2, Ptchd3, Gli1, Gli2, Gli3, Gsk3b, Smo, Sufu, Cdon, Cep76 (C18orf9), Fgf9, Fkbp8, Ift52, and OTX2.

Hair development pathway comprises Wnt, Shh, Notch, BMP and other signaling pathways responsible for hair follicle morphogenesis. Genes involved in hair development pathways include, but is not limited to Shh, Krt25, Dlx2, Prom1, S100a9, Vegfc, Ptgfr, Pdgfr1, Igfbp4, and Gli2.

Melanogenesis pathway comprises cell signal pathways responsible for melanocytes formation and maturation. Genes involved in melanogenesis pathways include, but is not limited to, Sox10, p300 family, Bcl2, MAP Kinases, POMC, Lef-1, Tyrp1, Trp1, Trp2, Syt4, Mlana, Pme1, Dct, Tyr, Sos1, Dbf4, Pax3, PIK3ca, Rps6kb1, Mlph, and Stx17.

In certain embodiments, the present disclosure is directed to methods of inducing hair growth in a subject in need thereof, the method comprising administering to a subject's hair follicle an inhibitor a therapeutically effective amount of a Jak/STAT inhibitor when the hair follicle is in mid-telogen phase or late-telogen phase. In certain embodiments, the Jak/STAT inhibitor is an antibody that specifically binds to a Jak/STAT protein or a fragment thereof; an antisense RNA, antisense DNA, an siRNA, an shRNA, a microRNA, or a variant or modification thereof that decreases expression of the gene that encodes the Jak/STAT protein; an antisense RNA, antisense DNA, an siRNA, an shRNA, a microRNA, or a variant or modification thereof that decreases expression of the Jak/STAT protein; a small molecule; or a combination thereof, the inhibitor administered to a subject's hair follicle when the hair follicle is in mid-telogen phase or late-telogen phase. In certain embodiments, the inhibitor administered to a subject's hair follicle when the hair follicle is in mid-telogen phase or late-telogen phase is ruxolitinib (INCB 018424). In certain embodiments, the inhibitor administered to a subject's hair follicle when the hair follicle is in mid-telogen phase or late-telogen phase is tofacitinib (CP690550). In a certain embodiments, the inhibitor administered to a subject's hair follicle when the hair follicle is in mid-telogen or late-telogen phase is ruxolitinib (INCB 018424), tofacitinib (CP690550), AG490, momelotinib (CYT387), partcitinib (SB1518), baricitinib (LY3009104), fedratinib (TG101348), BMS-911543, lestaurtinib (CEP-701), fludarabine, epigallocatechin-3-gallate (EGCG), baricitinib, momelotinib, pacritinib, peficitinib, ABT 494, AT 9283, decernmotinib, filgotinib, gandotinib, INCB 39110, PF 4965842, R348, AZD 1480, BMS 911543, cerdulatinib, INCB 052793, NS 018, C 410, CT 1578, JTE 052, PF 6263276, R 548, TG 02, lumbricus rebellus extract, ARN 4079, AR 13154, UR 67767, CS510, VR588, DNX 04042, or hyperforin or combinations thereof.

In certain embodiments, the present disclosure is directed to methods of promoting hair growth in a subject in need thereof, the method comprising administering to the subject a therapeutically effective amount of a Jak/STAT inhibitor. In certain embodiment, the Jak/STAT inhibitor is administered in a phase other than early telogen phase. In certain embodiments, the Jak/STAT inhibitor is administered in anagen phase. In certain embodiments, the Jak/STAT inhibitor is an antibody that specifically binds to a Jak/STAT protein or a fragment thereof; an antisense RNA, antisense DNA, an siRNA, an shRNA, a microRNA, or a variant or modification thereof that decreases expression of the gene that encodes the Jak/STAT protein; an antisense RNA, antisense DNA, an siRNA, an shRNA, a microRNA, or a variant or modification thereof that decreases expression of the Jak/STAT protein; a small molecule; or a combination thereof. In certain embodiments, the inhibitor is ruxolitinib (INCB 018424). In certain embodiments, the inhibitor is tofacitinib (CP690550). In a certain embodiments, the inhibitor is ruxolitinib (INCB 018424), tofacitinib (CP690550), AG490, momelotinib (CYT387), partcitinib (SB1518), baricitinib (LY3009104), fedratinib (TG101348), BMS-911543, lestaurtinib (CEP-701), fludarabine, epigallocatechin-3-gallate (EGCG), baricitinib, momelotinib, pacritinib, peficitinib, ABT 494, AT 9283, decernmotinib, filgotinib, gandotinib, INCB 39110, PF 4965842, R348, AZD 1480, BMS 911543, cerdulatinib, INCB 052793, NS 018, C 410, CT 1578, JTE 052, PF 6263276, R 548, TG 02, lumbricus rebellus extract, ARN 4079, AR 13154, UR 67767, CS510, VR588, DNX 04042, or hyperforin or combinations thereof.

### Method of Promoting Inductivity of Dermal Papilla

The present disclosure further relates to the use of one or more JAK/STAT proteins (e.g., Jak1, Jak2, Jak3, Tyk2, STAT1, STAT2, STAT3, STAT4, STAT5a, STAT5b, STAT6, OSM, gp130, LIFR, or OSM-Rβ) inhibitors (e.g., ruxolitinib (INCB 018424), tofacitinib (CP690550), AG490, momelotinib (CYT387), partcitinib (SB1518), baricitinib (LY3009104), fedratinib (TG101348), BMS-911543, lestaurtinib (CEP-701), fludarabine, epigallocatechin-3-gallate (EGCG), baricitinib, momelotinib, pacritinib, peficitinib, ABT 494, AT 9283, decernmotinib, filgotinib, gandotinib, INCB 39110, PF 4965842, R348, AZD 1480, BMS 911543, cerdulatinib, INCB 052793, NS 018, C 410, CT 1578, JTE 052, PF 6263276, R 548, TG 02, lumbricus rebellus extract, ARN 4079, AR 13154, UR 67767, CS510, VR588, DNX 04042, or hyperforin or combinations thereof), to promote inductivity of dermal papilla. In certain embodiments, the present disclosure is directed to methods of promoting inductivity of dermal papilla, the method comprising administering to a dermal papilla 3D sphere derived from a hair follicle of a subject a therapeutically effective amount of a Jak/STAT inhibitor. It is known in the art that in the hair follicle, hair follicle-derived dermal cells can interact with local epithelia and induce de novo hair follicles in a variety of hairless recipient skin sites (Higgins, et, al., Proc Natl Acad Sci USA 110, 19679 (Dec. 3, 2013), incorporated herein). It is also known in the art that human dermal papilla cells, when grown as 3D spheroids, are capable of inducing de novo hair follicles in human skin (Higgins, et, al.; Y. Zheng et al., J Invest Dermatol 124, 867 (May, 2005), incorporated herein). In certain embodiment, the dermal papilla 3D sphere is subsequently administered to the subject to treat androgenetic alopecia, telogen effluvium, alopecia areata, tinea capitis, alopecia totalis, hypotrichosis, hereditary hypotrichosis simplex, frontal fibrosing alopecia, cicatricial alopecia, lichen planopilaris, ring alopecia, scarring alopacia, nonscarring alopacia, chemotherapy induced alopecia, or alopecia universalis.

### 5.4 Pharmaceutical Compositions and Administration

In certain embodiments, the JAK-STAT inhibitor compositions of the present disclosure can be formulated as pharmaceutical compositions or pharmaceutical formulations by admixture with a pharmaceutically acceptable carrier or excipient. In certain embodiments, the pharmaceutical formulations can include a therapeutically effective amount of a JAK-STAT inhibitor and a physiologically acceptable diluent or carrier. In certain embodiments, the pharmaceutical composition can further include one or more additional therapeutic components and/or adjuvants.

In certain embodiments, the pharmaceutical formulation can be a solid dosage form. In certain embodiments, the solid dosage form can be a tablet or capsule.

In certain embodiments, the pharmaceutical formulation can be a liquid formulation. In certain embodiments, the liquid formulation can be an oral solution or oral suspension.

In certain embodiments, the pharmaceutical formulation can be a transdermal drug delivery system, e.g., a patch, cream, gel, and/or microemulsion.

In certain embodiments, the pharmaceutical formulation can include liposomes, nanoparticles, and/or other carriers. In certain embodiments, the pharmaceutical formulation can include an adjuvant or enhancer, e.g., an enzyme inhibitor.

In certain embodiments, the pharmaceutical formulation can be a direct infusion. In certain embodiments, the pharmaceutical formulation can be an implantable device.

Many methods can be used to introduce the formulations described herein, these include but are not limited to oral, topical, intradermal, intramuscular, intraperitoneal, intravenous, subcutaneous, and intra-pulmonary routes. All such routes are suitable for administration of these compositions, and can be selected depending on the patient and condition treated if there is a condition present, and similar factors by an attending physician. According to the desired route for administration, the compositions of the disclosure are prepared in the form of, for example, liquids, powders, aerosols, tablets, capsules, enteric coated tablets or capsules, or suppositories.

Selection of the appropriate dosage for the priming compositions of the present disclosure can be based upon the physical condition of the mammal, most especially including the general health and weight of the mammal. Such selection and upward or downward adjustment of the effective dose is within the skill of the art.

Pharmaceutical compositions of the present disclosure optionally further comprising sterile aqueous or non-aqueous solutions, suspensions, and emulsions. The composition can further comprise auxiliary agents or excipients, as known in the art. See, e.g., Berkow et al., eds., The Merck Manual, 15th edition, Merck and Co., Rahway, N.J. (1987); Goodman et al., eds., Goodman and Gilman's The Pharmacological Basis of Therapeutics, 8th edition, Pergamon Press, Inc., Elmsford, N.Y. (1990); Avery's Drug Treatment: Principles and Practice of Clinical Pharmacology and Therapeutics, 3rd edition, ADIS Press, LTD., Williams and Wilkins, Baltimore, Md. (1987); Osol, A., ed., Remington's Pharmaceutical Sciences, Mack Publishing Co, Easton, Pa. pp. 1324-1341 (1980); Katzung, ed. Basic and Clinical Pharmacology, Fifth Edition, Appleton and Lange, Norwalk, Conn. (1992), which references and references cited therein, are entirely incorporated herein by reference as they show the state of the art.

In certain embodiments, preparations for parenteral administration include sterile aqueous or non-aqueous solutions, suspensions, and/or emulsions, which can contain auxiliary agents or excipients known in the art. Examples of non-aqueous solvents are propylene glycol, polyethylene glycol, vegetable oils such as olive oil, and injectable organic esters such as ethyl oleate. Carriers or occlusive dressings can be used to increase skin permeability and enhance absorption. Liquid dosage forms for oral administration can generally comprise a liposome solution containing the liquid dosage form. Suitable forms for suspending liposomes include emulsions, suspensions, solutions, syrups, and elixirs containing inert diluents commonly used in the art, such as purified water. Besides the inert diluents, such compositions can also include adjuvants, wetting agents, emulsifying and suspending agents, or sweetening, flavoring, or perfuming agents. See, e.g., Berkow, infra, Goodman, infra, Avery's, infra, Osol, infra and Katzung, infra, which are incorporated in their entirety herein by reference.

In certain embodiments, a composition of the present disclosure, used for administration to an individual, can further comprise salts, preservatives, chemical stabilizers, buffers, adjuvants, or other substances which are desirable for improving the efficacy of the composition. Typically, stabilizers, adjuvants, and preservatives are optimized to determine the best formulation for efficacy in the target human or animal. Suitable exemplary preservatives include chlorobutanol potassium sorbate, sorbic acid, sulfur dioxide, propyl gallate, the parabens, ethyl vanillin, glycerin, phenol, and parachlorophenol. Suitable stabilizing ingredients which can be used include, for example, casamino acids, sucrose, gelatin, phenol red, N-Z amine, monopotassium diphosphate, lactose, lactalbumin hydrolysate, and dried milk.

Normally, the adjuvant and the composition are mixed prior to presentation, or presented separately, but into the same site of the mammal. Such adjuvants include, among others, MPL. (3-O-deacylated monophosphoryl lipid A; RIBI ImmunoChem Research, Inc., Hamilton, Mont.), mineral oil and water, aluminum hydroxide, Amphigen, Avridine, L121/squalene, D-lactide-polylactide/glycoside, pluronic plyois, muramyl dipeptide, killed Bordetella, saponins, such as Quil A or Stimulon QS-21 (Aquila Biopharmaceuticals, Inc., Framingham, Mass.) and cholera toxin (either in a wild-type or mutant form, e.g., wherein the glutamic acid at amino acid position 29 is replaced by another amino acid, preferably a histidine, in accordance with International Patent Application No. PCT/US99/22520, incorporated herein by reference). Additional examples of materials suitable for use in the compositions of the instant disclosure are provided in Osol, A., ed., Remington's Pharmaceutical Sciences, Mack Publishing Co, Easton, Pa. (1980), pp. 1324-1341, which reference is incorporated in its entirety herein by reference.

### 5.5 Methods of Monitoring Efficacy of Treatment

The present disclosure further relates to a method of assessing the efficacy of a therapy for inducing or promoting hair growth in a mammalian subject. In certain embodiments, the method comprises (a) determining a level of one or more hair growth biomarkers in a hair follicle sample obtained from the subject, and (b) determining the level of the one or more biomarkers in a hair follicle sample obtained from the subject, at one of more time points during the therapy, wherein the therapy is efficacious for inducing or promoting hair growth in the subject when there is a change of the one or more biomarkers in the second or subsequent samples, relative to the first sample. In certain embodiments, the biomarkers are selected from Wnt pathway, Shh pathway, hair development pathway, melanogenesis pathway, or any combination thereof. In certain embodiments, the biomarkers are selected from the group consisting of CD34, Lhx2, NFATc1, Axin2, FoxC1, OSMR, OSM, Jak3, FAS, Irf1, Ifnar1, Nr3c1, Stat5A, Il6st, Ptprc, Ghr, IL10ra, Il2rg, Pdgfra, Spfi1, Socs2, Stat5b, Crp, Il4, Prlr, Insr, IL2ra, Cebpd, Stat3, Jak1, Acvr2a, Sfrp4, Sox5, Cdh2, Fzd5, Wif1, Wnt2, Fzd8, Apc, Sox9, Ilk, Shh, Krt25, Dlx2, Prom1, S100a9, Vegfc, Ptgfr, Pdgfr1, Igfbp4, Gli2, Tyrp1, Syt4, Mlana, Pme1, Dct, Tyr, Sos1, Dbf4, Pax3, PIK3ca, Rps6kb1, Mlph, and Stx17.

A hair growth biomarker can be a nucleic acid or a peptide/protein. Methods for qualitatively and quantitatively detecting and/or determining the expression level of a nucleic acid biomarker, include, but are not limited to polymerase chain reaction (PCR), including conventional, qPCR and digital PCR, in situ hybridization (for example, but not limited to Fluorescent In Situ Hybridization (“FISH”)), gel electrophoresis, sequencing and sequence analysis, microarray analysis and other techniques known in the art.

In certain embodiments, the method of detection can be real-time PCR (RT-PCR), quantitative PCR, fluorescent PCR, RT-MSP (RT methylation specific polymerase chain reaction), PicoGreen™ (Molecular Probes, Eugene, Oreg.) detection of DNA, radioimmunoassay or direct radio-labeling of DNA. For example, but not by way of limitation, a nucleic acid biomarker can be reversed transcribed into cDNA followed by polymerase chain reaction (RT-PCR); or, a single enzyme can be used for both steps as described in U.S. Pat. No. 5,322,770, or the biomarker can be reversed transcribed into cDNA followed by symmetric gap ligase chain reaction (RT-AGLCR) as described by R. L. Marshall, et al., PCR Methods and Applications 4: 80-84 (1994).

In certain embodiments, quantitative real-time polymerase chain reaction (qRT-PCR) is used to evaluate mRNA levels of biomarker. The levels of a biomarker and a control mRNA can be quantitated in cancer tissue or cells and adjacent benign tissues. In certain embodiments, the levels of one or more biomarkers can be quantitated in a biological sample.

In a non-limiting embodiment, the method of detection of the present invention can be carried out without relying on amplification, e.g., without generating any copy or duplication of a target sequence, without involvement of any polymerase, or without the need for any thermal cycling. In certain embodiments, detection of the present invention can be performed using the principles set forth in the QuantiGene™ method described in U.S. application Ser. No. 11/471,025, filed Jun. 19, 2006, and is incorporated herein by reference.

In certain embodiments, in situ hybridization visualization can be employed, where a radioactively labeled antisense RNA probe is hybridized with a thin section of a biological sample, e.g., a biopsy sample, washed, cleaved with RNase and exposed to a sensitive emulsion for autoradiography. The samples can be stained with haematoxylin to demonstrate the histological composition of the sample, and dark field imaging with a suitable light filter shows the developed emulsion. Non-radioactive labels such as digoxigenin can also be used.

In certain non-limiting embodiments, evaluation of nucleic acid biomarker expression can be performed by fluorescent in situ hybridization (FISH). FISH is a technique that can directly identify a specific region of DNA or RNA in a cell and therefore enables visual determination of the biomarker expression in tissue samples. The FISH method has the advantages of a more objective scoring system and the presence of a built-in internal control consisting of the biomarker gene signals present in all non-neoplastic cells in the same sample. FISH is a direct in situ technique that can be relatively rapid and sensitive, and can also be automated. Immunohistochemistry can be combined with a FISH method when the expression level of the biomarker is difficult to determine by FISH alone.

In certain embodiments, expression of a nucleic acid biomarker can be detected on qPCR array, a DNA array, chip or a microarray. Oligonucleotides corresponding to the biomarker(s) are immobilized on a chip which is then hybridized with labeled nucleic acids of a biological sample, e.g., tumor sample, obtained from a subject. Positive hybridization signal is obtained with the sample containing biomarker transcripts. Methods of preparing DNA arrays and their use are well known in the art. (See, for example, U.S. Pat. Nos. 6,618,6796; 6,379,897; 6,664,377; 6,451,536; 548,257; U.S. Patent Application Nos. 20030157485 and Schena et al. 1995 Science 20:467-470; Gerhold et al. 1999 Trends in Biochem. Sci. 24, 168-173; and Lennon et al. 2000 Drug discovery Today 5: 59-65, which are herein incorporated by reference in their entirety). Serial Analysis of Gene Expression (SAGE) can also be performed (See, for example, U.S. Patent Application No. 20030215858).

In certain embodiments, to monitor a nucleic acid biomarker, mRNA can be extracted from the biological sample to be tested, reverse transcribed and fluorescent-labeled cDNA probes can be generated. The labeled cDNA probes can then be applied to microarrays capable of hybridizing to a biomarker, allowing hybridization of the probe to microarray and scanning the slides to measure fluorescence intensity. This intensity correlates with the hybridization intensity and expression levels of the biomarker.

Types of probes for detection of nucleic acid biomarkers include cDNA, riboprobes, synthetic oligonucleotides and genomic probes. The type of probe used will generally be dictated by the particular situation, such as riboprobes for in situ hybridization, and cDNA for Northern blotting, for example. In certain non-limiting embodiments, the probe is directed to nucleotide regions unique to the particular biomarker RNA. The probes can be as short as is required to differentially recognize the particular biomarker mRNA transcripts, and can be as short as, for example, 15 bases. Probes of at least 17 bases, 18 bases and 20 bases can also be used. In certain embodiments, the primers and probes hybridize specifically under stringent conditions to a nucleic acid fragment having the nucleotide sequence corresponding to the target gene. As herein used, the term “stringent conditions” means hybridization will occur only if there is at least 95% or at least 97% identity between the sequences.

The form of labeling of the probes can be any that is appropriate, such as the use of radioisotopes, for example, 32P and 35S, or fluorophores. Labeling with radioisotopes can be achieved, whether the probe is synthesized chemically or biologically, by the use of suitably labeled bases.

Methods for detecting and/or determining the level of a protein biomarker are well known to those skilled in the art, and include, but are not limited to, mass spectrometry techniques, 1-D or 2-D gel-based analysis systems, chromatography, enzyme linked immunosorbent assays (ELISAs), radioimmunoassays (RIA), enzyme immunoassays (EIA), Western Blotting, immunoprecipitation and immunohistochemistry. These methods use antibodies, or antibody equivalents, to detect protein, or use biophysical techniques. Antibody arrays or protein chips can also be employed, see, for example, U.S. Patent Application Nos. 2003/0013208; 2002/0155493, 2003/0017515 and U.S. Pat. Nos. 6,329,209 and 6,365,418, herein incorporated by reference in their entireties.

In certain non-limiting embodiments, a detection method for measuring protein biomarker expression includes the steps of: contacting a biological sample, e.g., a tissue sample, with an antibody or variant (e.g., fragment) thereof, which selectively binds the biomarker, and detecting whether the antibody or variant thereof is bound to the sample. The method can further include contacting the sample with a second antibody, e.g., a labeled antibody. The method can further include one or more washing steps, e.g., to remove one or more reagents.

In certain non-limiting embodiments, Western blotting can be used for detecting and quantitating biomarker protein expression levels. Cells can be homogenized in lysis buffer to form a lysate and then subjected to SDS-PAGE and blotting to a membrane, such as a nitrocellulose filter. Antibodies (unlabeled) can then brought into contact with the membrane and assayed by a secondary immunological reagent, such as labeled protein A or anti-immunoglobulin (suitable labels including 125I, horseradish peroxidase and alkaline phosphatase). Chromatographic detection can also be used. In certain embodiments, immunodetection can be performed with antibody to a biomarker using the enhanced chemiluminescence system (e.g., from PerkinElmer Life Sciences, Boston, Mass.). The membrane can then be stripped and re-blotted with a control antibody specific to a control protein, e.g., actin.

Immunohistochemistry can be used to detect the expression and/or presence of a biomarker, e.g., in a biopsy sample. A suitable antibody can be brought into contact with, for example, a thin layer of cells, followed by washing to remove unbound antibody, and then contacted with a second, labeled, antibody. Labeling can be by fluorescent markers, enzymes, such as peroxidase, avidin or radiolabeling. The assay can be scored visually, using microscopy, and the results can be quantitated. Machine-based or autoimaging systems can also be used to measure immunostaining results for the biomarker.

Various automated sample processing, scanning and analysis systems suitable for use with immunohistochemistry are available in the art. Such systems can include automated staining (see, e.g., the Benchmark system, Ventana Medical Systems, Inc.) and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.).

Labeled antibodies against biomarkers can also be used for imaging purposes, for example, to detect the presence of a biomarker in cells of a subject. Suitable labels include radioisotopes, iodine (125I, 121I), carbon (14C), sulphur (35S), tritium (3H), indium (112In), and technetium (99mTc), fluorescent labels, such as fluorescein and rhodamine, and biotin. Immunoenzymatic interactions can be visualized using different enzymes such as peroxidase, alkaline phosphatase, or different chromogens such as DAB, AEC or Fast Red. The labeled antibody or antibody fragment will preferentially accumulate at the location of cells which contain a biomarker. The labeled antibody or variant thereof, e.g., antibody fragment, can then be detected using known techniques.

Antibodies include any antibody, whether natural or synthetic, full length or a fragment thereof, monoclonal or polyclonal, that binds sufficiently strongly and specifically to the biomarker to be detected. An antibody can have a Kd of at most about 10-6M, 10-7M, 10-8M, 10-9M, 10-10M, 10-11M and 10-12M. The phrase “specifically binds” refers to binding of, for example, an antibody to an epitope or antigen or antigenic determinant in such a manner that binding can be displaced or competed with a second preparation of identical or similar epitope, antigen or antigenic determinant.

Antibodies, and derivatives thereof, that can be used encompass polyclonal or monoclonal antibodies, synthetic and engineered antibodies, chimeric, human, humanized, primatized (CDR-grafted), veneered or single-chain antibodies, phase produced antibodies (e.g., from phage display libraries), as well as functional binding fragments, of antibodies. For example, antibody fragments capable of binding to a biomarker, or portions thereof, including, but not limited to, Fv, Fab, Fab′ and F(ab′)2 fragments, can be used. Such fragments can be produced by enzymatic cleavage or by recombinant techniques.

In certain non-limiting embodiments, agents that specifically bind to a polypeptide other than antibodies are used, such as peptides. Peptides that specifically bind can be identified by any means known in the art, e.g., peptide phage display libraries. Generally, an agent that is capable of detecting a biomarker polypeptide, such that the presence of a biomarker is detected and/or quantitated, can be used. As defined herein, an “agent” refers to a substance that is capable of identifying or detecting a biomarker in a biological sample (e.g., identifies or detects the mRNA of a biomarker, the DNA of a biomarker, the protein of a biomarker).

In addition, a biomarker can be detected using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See, for example, U.S. Patent Application Nos. 2003/0199001, 2003/0134304, 2003/0077616, which are herein incorporated by reference in their entireties.

Mass spectrometry methods are well known in the art and have been used to quantify and/or identify biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

Detection of the presence of a biomarker or other substances will typically involve detection of signal intensity. This, in turn, can reflect the quantity and character of a polypeptide bound to the substrate. For example, in certain embodiments, the signal strength of peak values from spectra of a first sample and a second sample can be compared (e.g., visually or by computer analysis), to determine the relative amounts of a particular biomarker. Software programs such as the Biomarker Wizard program (Ciphergen Biosystems, Inc., Fremont, Calif.) can be used to aid in analyzing mass spectra.

Additional methods for determining nucleic acid and/or protein biomarker expression in samples are described, for example, in U.S. Pat. No. 6,271,002; U.S. Pat. No. 6,218,122; U.S. Pat. No. 6,218,114; and U.S. Pat. No. 6,004,755; and in Wang et al, J. Clin. Oncol., 22(9): 1564-1671 (2004); and Schena et al, Science, 270:467-470 (1995); all of which are incorporated herein by reference in their entireties.

### 5.5 Kits

The present disclosure further relates to a kit for inducing or promoting hair growth in a mammalian subject. In certain embodiments, the kit comprises (a) a Jak1, a Jak2, a Jak3, a Tyk2, a STAT1, a STAT2, a STAT3, a STAT4, a STAT5a, a STAT5b, a STAT6, an OSM, a gp130, an LIFR, and/or an OSM-Rβ inhibitor; and (b) a pharmaceutically acceptable carrier. In certain embodiments, the Jak/STAT inhibitor is an antibody that specifically binds to a Jak/STAT protein or a fragment thereof; an antisense RNA, antisense DNA, an siRNA, an shRNA, a microRNA, or a variant or modification thereof that decreases expression of the gene that encodes the Jak/STAT protein; an antisense RNA, antisense DNA, an siRNA, an shRNA, a microRNA, or a variant or modification thereof that decreases expression of the Jak/STAT protein; a small molecule; or a combination thereof. In certain embodiments, the inhibitor is ruxolitinib (INCB 018424). In certain embodiments, the inhibitor is tofacitinib (CP690550). In a certain embodiments, the inhibitor is ruxolitinib (INCB 018424), tofacitinib (CP690550), AG490, momelotinib (CYT387), partcitinib (SB1518), baricitinib (LY3009104), fedratinib (TG101348), BMS-911543, lestaurtinib (CEP-701), fludarabine, epigallocatechin-3-gallate (EGCG), baricitinib, momelotinib, pacritinib, peficitinib, ABT 494, AT 9283, decernmotinib, filgotinib, gandotinib, INCB 39110, PF 4965842, R348, AZD 1480, BMS 911543, cerdulatinib, INCB 052793, NS 018, C 410, CT 1578, JTE 052, PF 6263276, R 548, TG 02, lumbricus rebellus extract, ARN 4079, AR 13154, UR 67767, CS510, VR588, DNX 04042, or hyperforin or combinations thereof.

### 6. EXAMPLES

The following example is put forth so as to provide those of ordinary skill in the art with a complete disclosure and description of how to make and use the presently disclosed subject matter, including, but not limited to compositions and methods for the induction of hair growth by the administration of inhibitors of the JAK-STAT pathway. The following example is not intended to limit the scope of the presently disclosed subject matter. It is understood that various other embodiments may be practiced, given the general description provided above.

### 6.1 Example 1: Pharmacologic Inhibition of JAK-STAT Signaling Promotes Hair Growth

### Results

JAK-STAT Inhibition Results in Rapid Onset of Hair Growth in Mice.

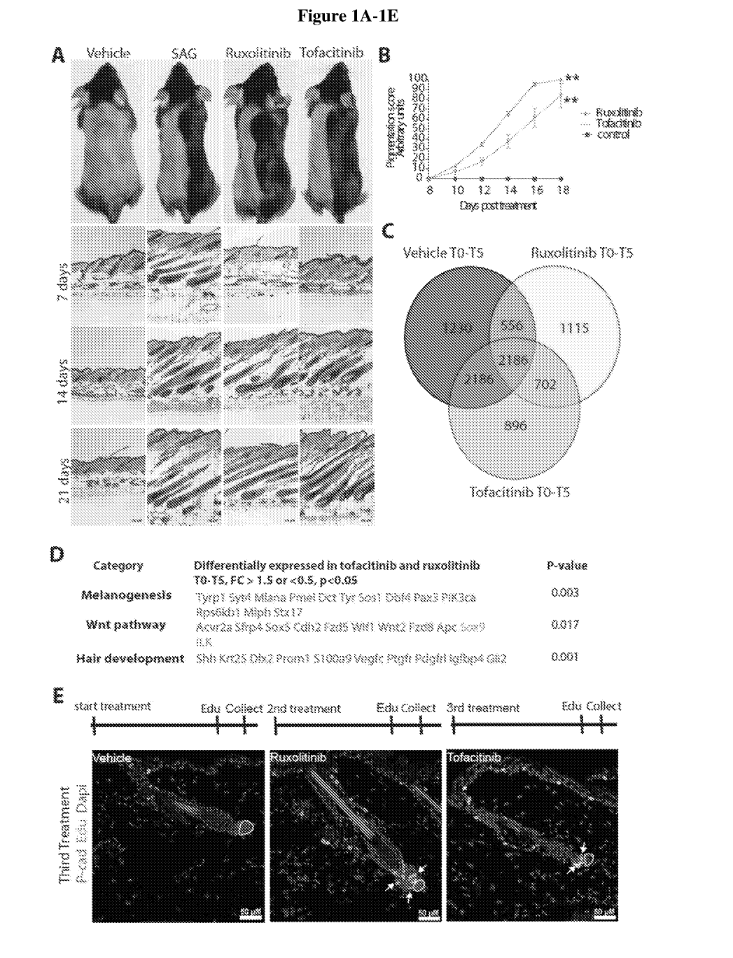

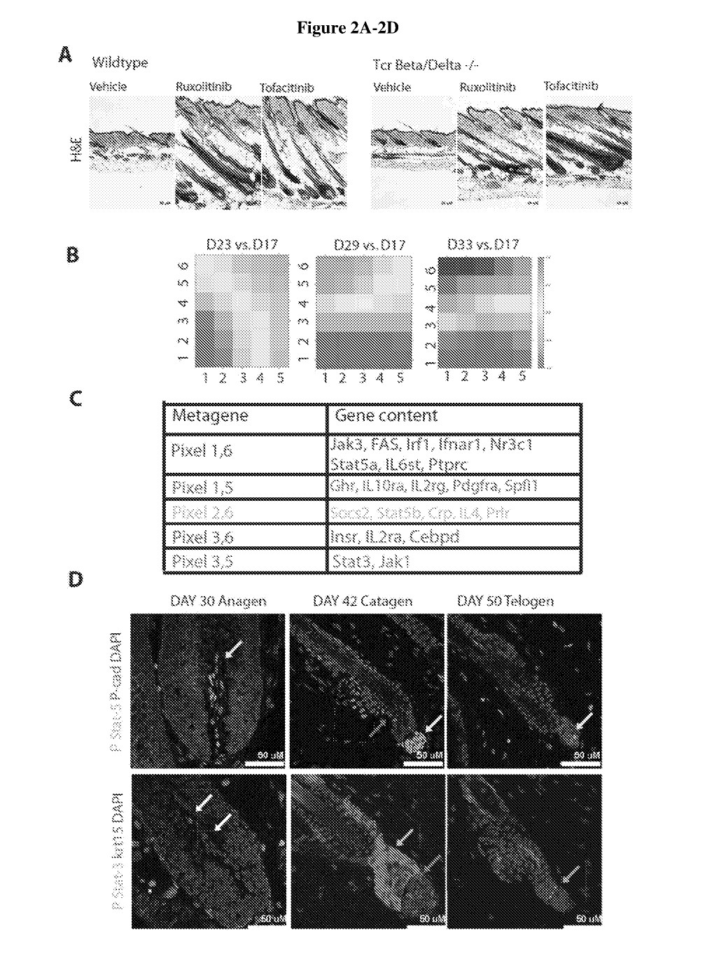

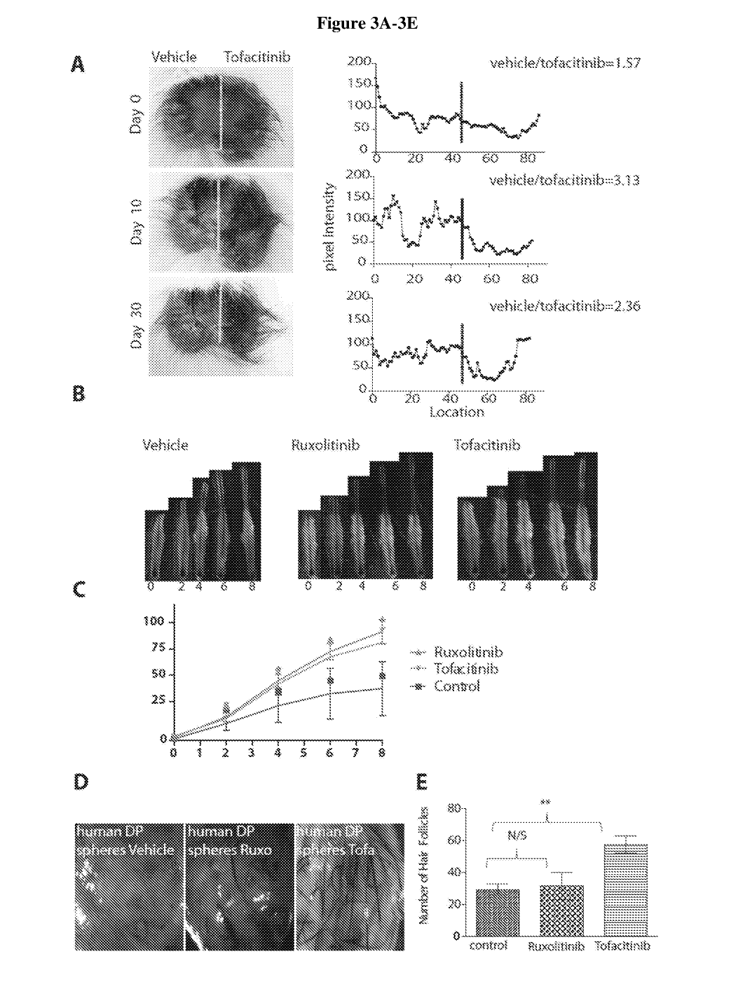

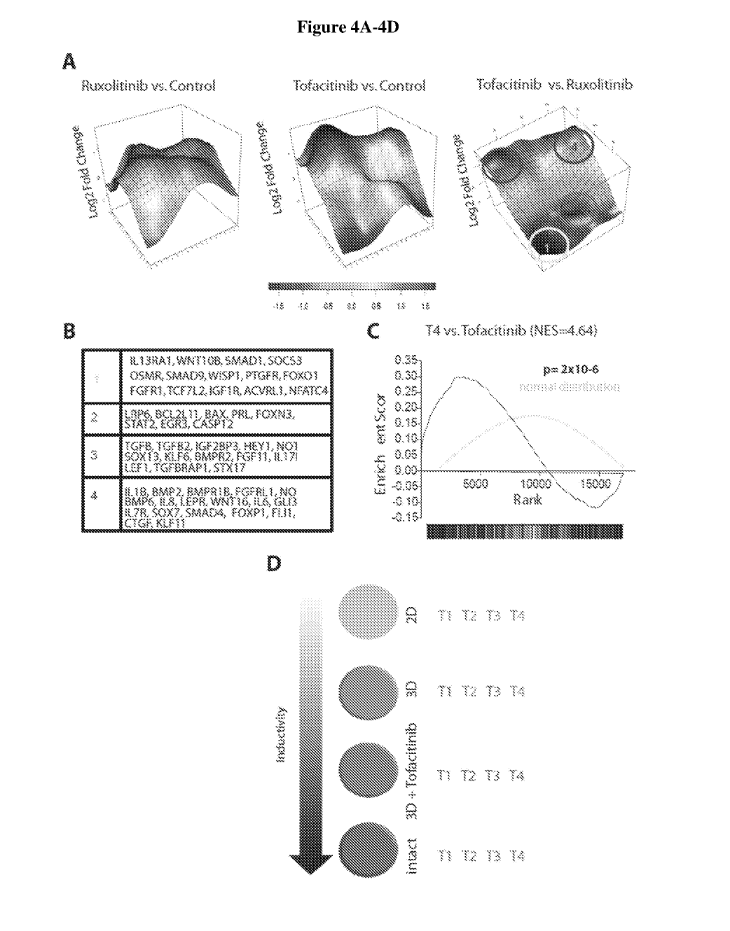

C57/B6 mice in telogen were first treated for three weeks on half of the dorsal back with either vehicle control (negative control, left side), a sonic hedgehog (Shh) agonist previously shown to promote anagen initiation (2) (positive control), or several small molecule inhibitors of the JAK-STAT pathway, including tofacitinib (JAK1/3>JAK2>TYK2)(3) and ruxolitinib (JAK1/2>Tyk2>JAK3)(4-7) (right side) (FIG. 1A). As expected, entry into anagen was evident within 7 days of treatment with the Shh agonist, whereas vehicle-treated mice remained in telogen for the duration of the experiment. Intriguingly, treatment with the JAK inhibitors resulted in rapid re-entry into the hair cycle, with kinetics similar to the Shh agonist (FIG. 1A). To examine a direct effect on stem cell activation, mice in telogen was treated with tofacitinib or ruxolitinib for a short period of 4 days. Significant proliferation was noted within the hair germ compartment (P-cad+) of drug-treated HFs (FIG. 5A), indicating activation of a progenitor population. Quantification of the effects of drug treatment on skin homeostasis demonstrated that drug-induced hair growth recapitulates normal hair growth (FIG. 5B). Taken together, these data suggest that local inhibition of the JAK-STAT pathway results in rapid onset of hair growth.

Effects of JAK-STAT Inhibition are Dependent on the Duration of Telogen.

The first postnatal hair cycle in C57/B6 mice follows a precise temporal progression. Regeneration (anagen re-entry) thereafter occurs in spontaneous, patchy waves, starting around 12-13 weeks of age (8, 9). Treatment of mice in telogen with JAK inhibitors consistently yielded early and homogenous hair growth (FIG. 1A), however, the duration of treatment required to re-initiate anagen was inexplicably variable. To address this issue, mice in early telogen (7 weeks) or mid telogen (8.5 weeks) were treated. The treatment resulted in no growth in the 7-week-old mice, whereas 8.5-week-old mice exhibited rapid onset of anagen (FIG. 5C). To ensure that mice treated at 8.5 weeks of age were still at the refractory stage of telogen which arrests the propagation of anagen re-entry (9, 10), hair was plucked from mice at 7 and 8.5 weeks and hair growth post plucking followed a similar kinetics in both time points (FIG. 5C). Notably, longer treatment of 7-week-old mice for 18-21 days eventually induced hair growth, but only after the treated mice had reached 8.5 weeks of age (FIG. 5D). This finding implies that JAK inhibition cannot override the quiescence-promoting microenvironment at the early stages of telogen (9, 10), but is sufficient to promote hair growth at a later stage in telogen.

To demonstrate the robustness and reproducibility of hair growth resulting from treatment with JAK inhibitors at 8.5 weeks of age, skin darkening was used as a proxy for hair growth in C57/B6 mice (11). Indeed, ˜90% of 8.5 week old mice treated with ruxolitinib or tofacitinib for 5 days displayed skin darkening and hair growth within 10 days of starting treatment, while no hair growth was evident in control-treated mice (P<0.0001 for ruxolitinib treatment and p=0.04 for tofacitinib treatment) (FIGS. 1B and 5E).

Hair Growth Following JAK-STAT Inhibition Mimics Normal Anagen Initiation by Activating the Wnt and Shh Signaling Pathways.

To examine whether anagen initiation post treatment with JAK inhibitors is molecularly similar to normal anagen initiation, microarray experiments were performed on 8.5-week-old mice treated with vehicle control, ruxolitinib or tofacitinib for 4 days, a time point at which proliferation in the hair germ has begun but hair growth is not yet evident (FIG. 5A). Comparison of the differentially expressed gene lists between whole skin harvested at day 0 (T0) and day 4 (T5) of treatment revealed a subset of genes regulated by both JAK inhibitors (FIG. 1C). Pathway analysis using Ingenuity Pathway Analysis (IPA) software showed that melanogenesis and the Wnt pathway were enriched in both ruxolitinib and tofacitinib treatments, but not in the vehicle treatment. Further analysis of differentially expressed genes in both drug treatments identified other important hair cycle regulators, such as Shh and Prom1 (2, 12-16), as significantly upregulated in JAK inhibitors treated skin (FIG. 1D). Differential regulation of key genes was verified by qPCR (FIG. 6A). Since upregulation of Wnt and Shh pathways are central for anagen initiation and for activation of melanocyte stem cells (16, 17), these findings suggest that blockade of JAK-STAT signaling allows for normal progression of the hair cycle. Further analysis of genes regulated by only one of the drug treatments revealed a distinct molecular signature (FIGS. 6B & 6C). Ruxolitinib treatment enriched for the mTOR and NfkB pathways, both previously shown to be involved in hair cycle regulation (12, 18-20), whereas tofacitinib treatment enriched for pathways involved in cell motility and migration, such as Rho and integrin signaling. Interestingly, STAT3−/− keratinocytes were previously shown to be deficient in migration in response to stimuli (21, 22) suggesting that the JAK-STAT pathway is essential for cellular motility in the transition between telogen and anagen.

JAK-STAT Inhibition Causes Activation of Hair Follicle Progenitor Cells.

To investigate the cellular mechanisms responsible for HF activation following JAK-STAT inhibition, mice in telogen were treated with ruxolitinib, tofacitinib or vehicle control and harvested skin 5 hours after the first, second and third treatments (FIG. 1E, schematic). EDU was injected daily, 1 hour before harvesting each time point, and skin samples were analyzed for presence of EDU+ (proliferating) cells. Edu+ cells are clearly visible within the hair germ (P-cadherin+) compartment after three treatments in both ruxolitinib and tofacitinib treated skin but not in control treated skin (FIG. 1E). This finding suggests that activation of the HF after JAK-STAT inhibition follows the normal anagen initiation kinetics in which hair germ proliferation precedes bulge stem cell proliferation (23). Taken together, the data implies that JAK-STAT signaling normally acts to prevent anagen reentry, and that JAK blockade relieves this inhibition to allow for normal hair cycle progression.

Hair Inducing Effects of JAK Inhibition are not Dependent on the Activity of Lymphocytes.

The JAK-STAT pathway is known to play a prominent role in T cell biology (24), and the HF microenvironment contains a substantial population of resident and migrating T cells. Recent studies suggested that gamma delta T cells secrete factors that regulate HF neogenesis in mice, as well as aspects of hair cycling (25, 26). Moreover, the inventors previously demonstrated that in AA, JAK-STAT inhibitors act to clear cytotoxic T cells from the HF microenvironment, an essential process for onset of hair regrowth.

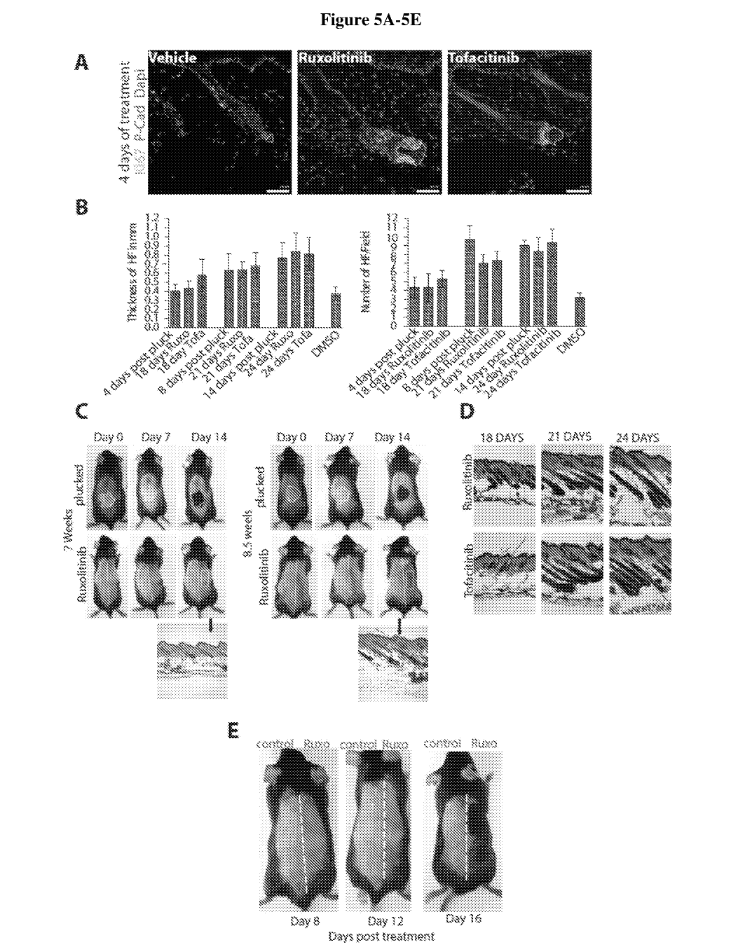

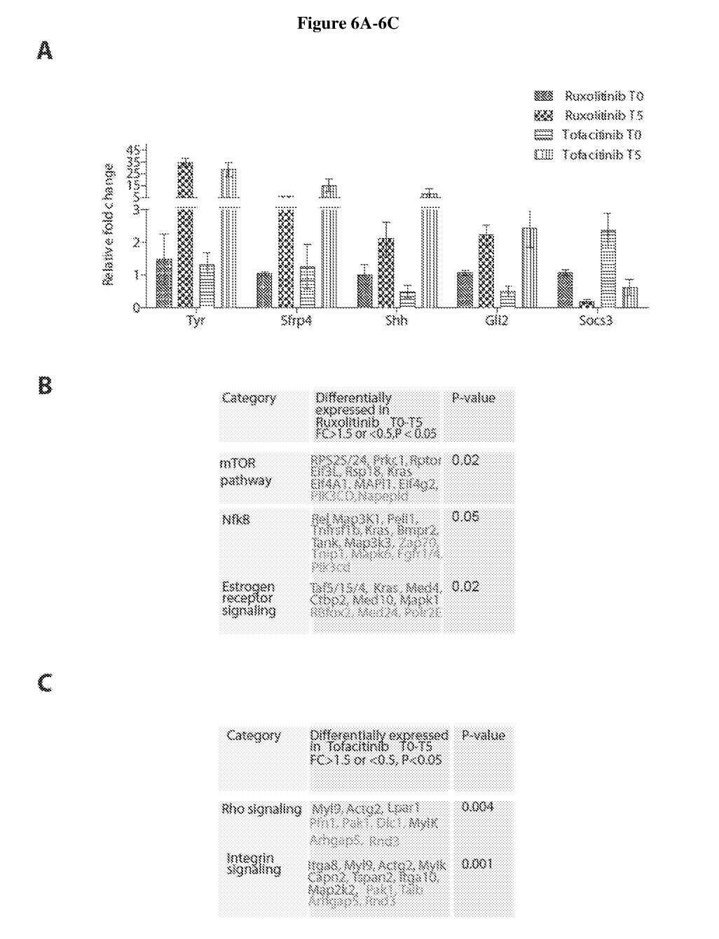

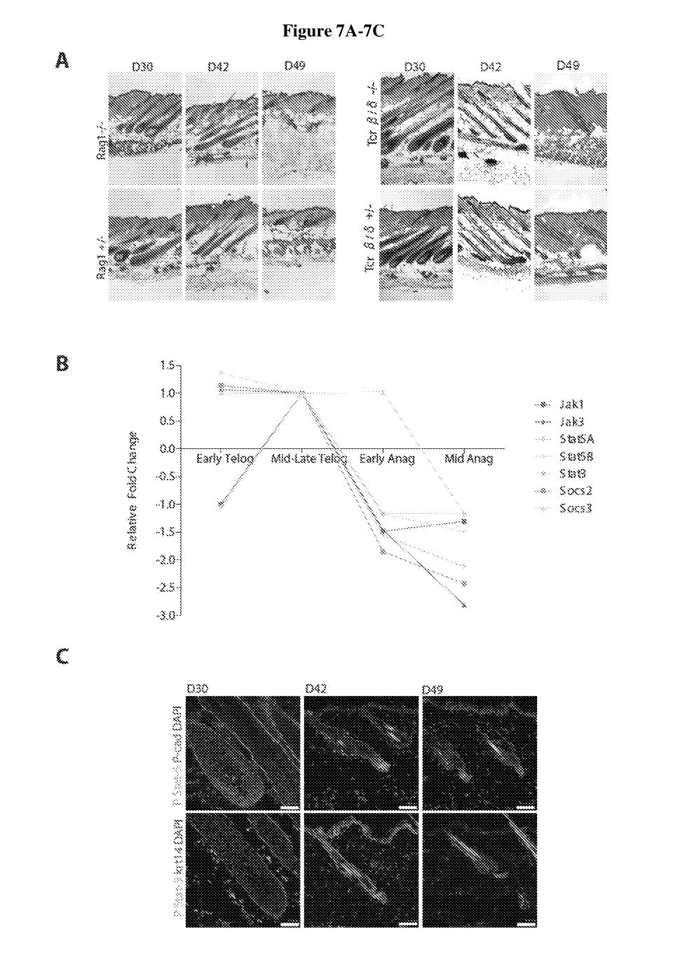

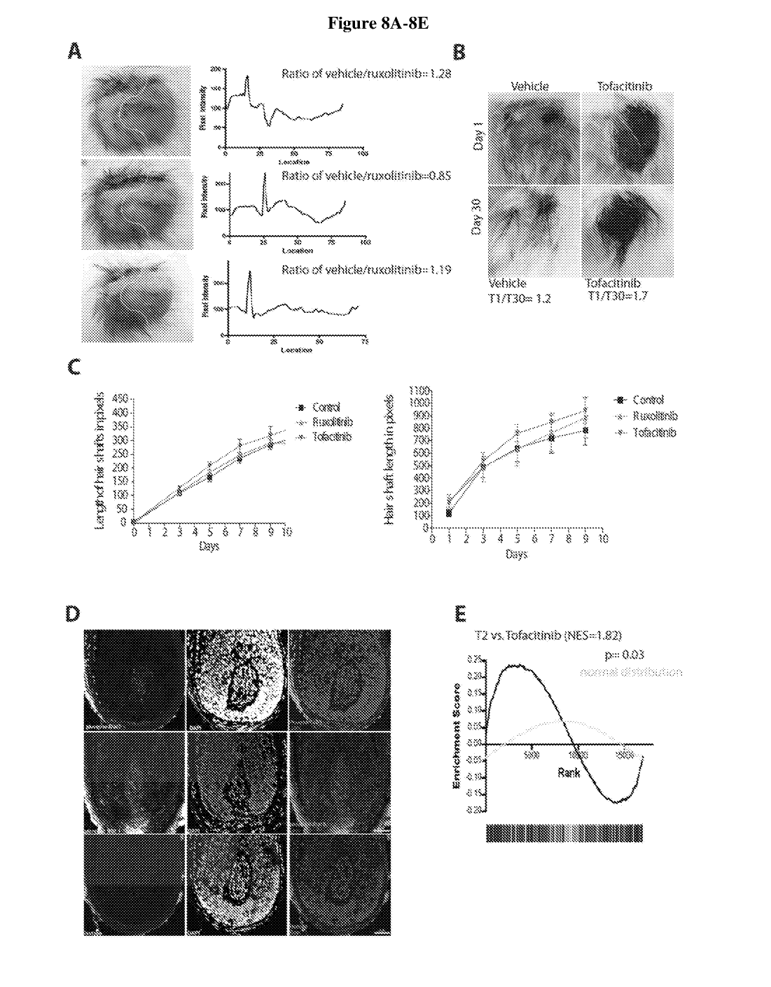

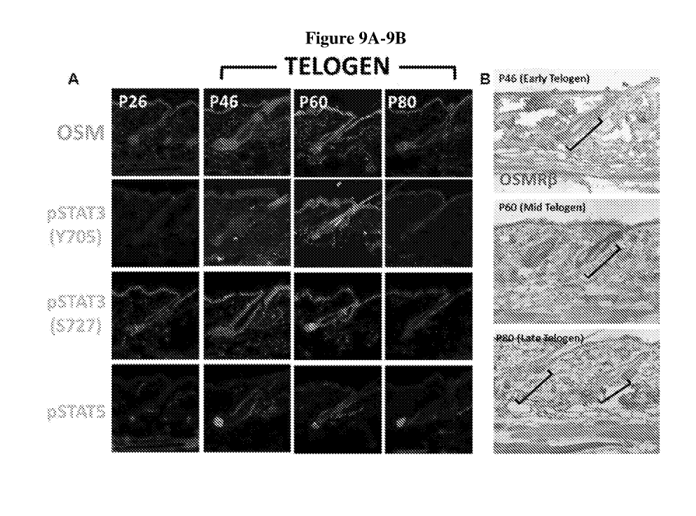

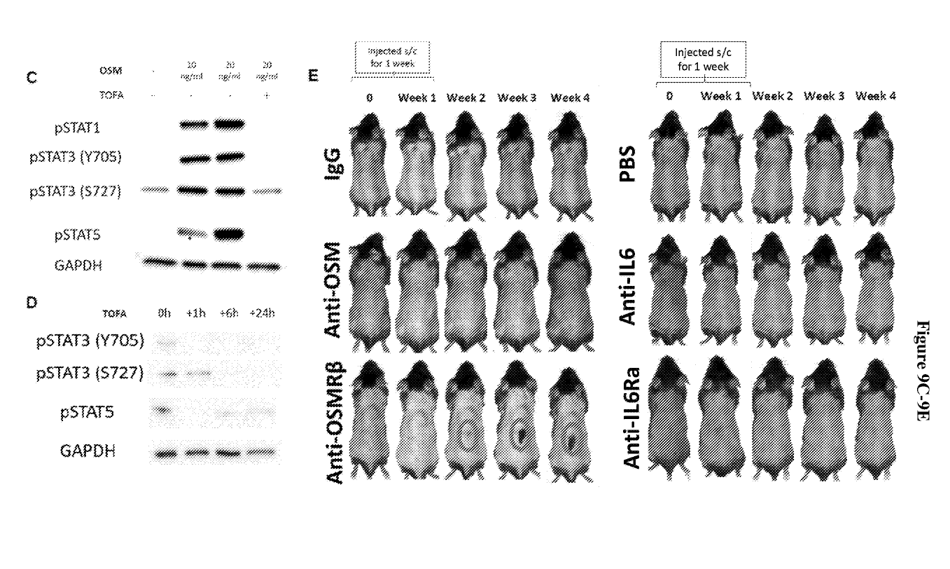

To evaluate if normal hair growth following treatment with JAK inhibitors is mediated by T cells, the effects of topical drug treatments were examined on two different lymphocyte-deficient mouse models. Rag1−/− mice, deficient in B and T cells, and Tcr β/δ−/− mice, deficient in migrating and resident T cells, were largely indistinguishable from controls in terms of their ability to enter the hair cycle (FIG. 7A), as well as their response to drug treatment (FIG. 2A). This suggests that the hair inducing effects of JAK inhibitors in normal skin are not dependent on the activity of lymphocytes. While the inventors have not ruled out the effects of JAK-STAT inhibitors on other immune cells known to affect the hair cycle such as macrophages (27), or perturbed by JAK inhibition, such as myeloid dendritic cells (28), this result suggests that the anagen inducing effects of JAK-STAT inhibition likely represent a hair-intrinsic property.

The JAK-STAT Pathway is Dynamically Regulated Across the Hair Cycle.

To examine the kinetics of the JAK-STAT pathway in HF development and cycling, changes in gene expression were examined in whole skin during the transition from anagen to telogen (29). Utilizing a qPCR array, the expression of genes related to the JAK-STAT pathway was measured. Dynamic changes in gene expression were visualized using Gene Expression Dynamics Inspector (GEDI), an algorithm that clusters expression values for each gene on the qPCR array into metagenes categories based on similarities in their temporal expression profiles, placing them into 1 of 30 places on a 5×6 grid. Comparison between catagen (D17) and early anagen (D29) revealed a cluster of metagenes that become repressed as the hair cycle progressed (boxed pixels, FIG. 2B). Investigating the content of the repressed metagenes revealed that key members of the JAK-STAT pathway such as STAT5A/B, STAT3, Jak1, Jak3 and Socs2/3 were expressed at high levels in catagen and telogen and were repressed in early anagen (FIGS. 2B, 2C & 7B).

Immunofluorescence studies of HF in anagen, catagen and telogen confirmed that the activated (phosphorylated) STAT3, is expressed in the dermal papilla (DP), some extra-follicular cells and in the proliferating cells of the basal epidermis (FIGS. 2D and 7C). In catagen and telogen, phospho-STAT3 can also be detected in cells of the hair germ. Activated phospho-STAT5 is strongly expressed in the DP throughout the hair cycle, with expression peaking during catagen, where it can also be detected in the bulge (FIG. 2D). The striking expression pattern of phospho-STAT5 in key HF stem cell compartments in telogen underscores a potentially important role in regulation of quiescence.

Tofacitinib Treatment Promotes Growth of Human Hair Follicles.

The effects of JAK inhibition on hair growth were examined in human tissues. In contrast to mice, human scalp HFs grow asynchronously and 90% of them are in the anagen phase of the hair cycle at any given time (34). Therefore, it is extremely difficult to assess the transition between telogen to anagen in humans, and analysis is confined to measuring the rate of hair fiber growth. Human fetal scalp skin was grafted onto Scid mice and allowed to recover for at least 6 weeks. Each graft was then treated with daily topical application of vehicle control on one side, and either tofacitinib (FIG. 3A) or ruxolitinib (FIG. 8A) on the other side. Small HFs were already present within the grafts, and therefore the inventors tested the effect of JAK inhibition on ongoing development of fetal HFs to terminal hairs and not on HF neogenesis. Tofacitinib treatment resulted in denser HF growth as compared to controls, suggesting that tofacitinib treatment increased the rate of hair elongation. To quantify this outcome, the inventors measured the intensity of pigmentation as a proxy for density of dark hairs on grafted white mice, and show that the ratio of tofacitinib/vehicle increased with days of treatment. Experiments were performed with skin derived from different donors with similar results (FIG. 8B).

The effects of JAK-inhibition on hair shaft elongation were further analyzed using the widely utilized human HF organ culture model. Individual HFs were microdissected from human adult scalp tissue, and cultured with vehicle control, ruxolitinib and tofacitinib (FIG. 3B). The treatment with JAK inhibitors significantly increased length of hair shafts when treated with ruxolitinib and tofacitinib, indicating a positive effect on the rate of hair elongation (P=0.023, and P=0.025 for tofacitinib and ruxolitinib, respectively). Experiments with HFs from 2 additional donors yielded a similar trend (FIG. 8C). Taken together, the data suggest that JAK-STAT inhibition promotes faster hair fiber growth in the organ culture model.

Tofacitinib Treatment Promotes Inductivity of Dermal Papilla.

Since phospho-STAT5 is strongly expressed in mouse DP in catagen and telogen (FIG. 2D), phospho STAT 3 is confirmed to be present in the dermal sheath and DP of human HFs in anagen, and phospho STAT 5 expression is weak but present in the top portion of the DP (FIG. 8D).

The inventors recently demonstrated that growing human DP cells in 3D spheres improves their capacity to induce HF growth (35). To examine the effects of JAK inhibition on the potency of HF induction, human DP spheres were cultured with vehicle control, ruxolitinib or tofacitinib, and then combined with neonatal mouse keratinocytes and injected into nude mice. This patch assay has been shown to recapitulates HF morphogenesis, and serves to evaluate trichogenic capacity (36). Tofacitinib-treated human DP spheres induced larger and significantly greater numbers of HFs overall (P=0.00013) (FIG. 3D), suggesting that the inductivity of human DP is enhanced by inhibition of JAK1/3 signaling.

Tofacitinib Treatment Promotes Hair Growth by Targeting Genes Enriched in Fully Inductive Dermal Papillae.

To investigate the mechanisms by which tofacitinib treatment improves DP inductivity, microarray experiments were performed on control, ruxolitinib and tofacitinib treated DP spheres. Log 2 fold changes in gene expression were used to generate GEDI plots. To analyze relevant changes in gene expression, the inventors compared ruxolitinib treatment (which did not confer enhanced inductivity) to controls, tofacitinib treatment (which did enhance inductivity) to controls, and ruxolitinib and tofacitinib treatments to each other. This allowed examination of gene expression changes resulting from JAK inhibition provided by both drugs, and focus on changes that were unique to tofacitinib treatment. The GEDI algorithm clustered differentially expressed transcripts into metagenes based on their similar expression pattern across all microarrays. Data is presented in 3D form, where the Z-axis and colors correspond to changes in gene expression, and the X, Y axes correspond to coordinates of GEDI metapixels, plotted on a 18×19 grid (FIG. 4A). Based on the topography of the graphs, four regions of interests were selected (regions 1-4).

Interestingly, amongst genes that were repressed in both treatments but lower in tofacitinib treatment (region 1) were receptors known to be involved in modulation of DP inductivity, such as FGFR1, ACVRL1, IGFR1, OSMR and PTGFR (32, 37-41). In the region including genes that were upregulated by ruxolitinib treatment but downregulated by tofacitinib treatment, pro-apoptotic genes were identified, such as BAX, BCL2L11 and CASP12 (region 2). Genes upregulated by tofacitinib treatment (regions 3 and 4) included members of the TGFβ pathway and the BMP pathway, previously shown to play a crucial role in dermal papilla inductivity (40-44). Key regulators of the WNT pathway, such as LEF1, known to regulate dermal-epidermal interactions (45, 46) and members of the NOTCH pathway known to control HF fate (47, 48) were overrepresented in tofacitinib treatment. Taken together, this suggests that tofacitinib treatment promotes inductivity by regulating both the inductive function and the survival of cultured dermal papillae.

To directly compare tofacitinib treatment with published work on DP inductivity, the inventors turned to the previous study which investigated the molecular differences between freshly isolated human DP (which maintain their capacity to induce hair growth), cultured DP (which lose their inductive potential) and cultured dermal spheroids (which have restored inductive potential), and identified unique gene signatures associated with each state (35). These genes clustered by coexpression into four categories referred to as territories (T1-T4). T1 and T3 were found to contain genes whose expression was deregulated in culture and restored by growth in spheroids, whereas T2 and T4 included genes whose expression was not restored by spheroid culture (35). The genes within T2 (upregulated in cultured cells and not restored by sphere formation) and T4 (downregulated in cultured cells and not restored by sphere formation) represent molecular signatures that appear to be required for fully inductive DP.

Since tofacitinib treatment enhanced hair growth, the inventors assayed whether the expression of genes within T1-T4 is enriched in a statistically significant manner, when comparing tofacitinib-treated to untreated samples. Gene set enrichment analysis (49) on genes differentially expressed in these treatment arms, ranked by p-value from highest to lowest expression, revealed that territory T4 was significantly enriched in tofacitinib treated spheres (P=2×10⁻⁶ gene list provided in Table 3) (FIGS. 4C & 4D). T2 was also enriched by tofacitinib treatment, however the statistical overlap was only marginally significant (P=0.03) (FIG. 8E). Not all genes within T2 and T4 changed in the manner predicted by the previous study, suggesting that restoration of inductivity by tofacitinib treatment may not be complete. Tofacitinib treatment did upregulate the expression of genes such as LEF1, WIF1 and CD133, all known regulators of hair growth (40, 41), providing a mechanistic explanation as to why tofacitinib treatment was successful in improving inductivity. Interestingly, analysis of the enrichment score of all JAK STAT genes identified by the qPCR microarray utilized in FIG. 2B within T2 and T4, uncovered a highly statistically significant overlap (1.45×10⁻⁷, gene list provided in Table 1), suggesting that there is a major JAK-STAT component to the T2 and T4 inductive signature

### Discussion

The findings presented herein demonstrate that inhibition of JAK-STAT signaling promotes hair growth. These findings are consistent with the association of JAK-STAT with an anti-hair growth pattern (29), and with evidence for the role of STAT3 in progression of the normal hair cycle in adult mice (21, 22). Moreover, recent studies have shown that increased JAK-STAT signaling in aged mice inhibits HF stem cell function in vitro (50) and that STAT5 signaling controls HF stem cell quiescence during pregnancy and lactation (51).

The observation that inhibition of JAK-STAT signals can promote activation or differentiation of stem cells/progenitor cells is not unique to the HF. Loss of STAT5 in hematopoietic stem cells induces exit from a quiescent state, leading to increased bone marrow repopulating capacity after irradiation (52). Inhibition of JAK-STAT signaling improves skeletal muscle regeneration in aged mice by promoting symmetric satellite cell expansion and reduced commitment to myogenesis (53, 54). Therefore, the role of JAK-STAT signaling in promoting quiescence may represent a generalized mechanism in adult stem cell populations.

In this study, the inventors observed that JAK inhibitors-mediated hair growth was independent of T lymphocyte function, and likely represents a hair-intrinsic property. The inventors recently demonstrated that treatment of AA patients with JAK inhibitors led to hair regrowth due in part to clearance of the CD8+NKG2D+ cytotoxic T cell infiltrate, but did not rule out a direct effect on the HF (1). These two findings are reconciled when considering hair growth in AA patients as a two-step mechanism: first, the T-cell mediated immune attack on epithelial cells must be eliminated, and second, anagen growth must be reinitiated (55). The inventors observed that topical treatment with JAK inhibitors resulted in more robust hair growth than systemic treatment in AA, specifically because it increases the local concentration of drug in the HF microenvironment, allowing both actions to occur. In unaffected individuals or in normal mice, treatment with JAK inhibitors may be sufficient to restart the hair cycle (in mice) or promote hair growth (in humans).

In mice, suppression of JAK signaling activates a pro-growth/anti-quiescence signal during telogen (9, 56, 57), thereby allowing re-entry into anagen.

The inventors observed that activation of the hair germ compartment, containing progenitor cells, was an early event in JAK-inhibition mediated hair growth and noted that pathways activated in early anagen are upregulated after JAK inhibition. These results suggest that JAK-inhibition mediated hair growth follows the normal proliferative pattern of homeostatic hair cycling (16, 23).

The date showed that anagen reentry following drug treatment occurs when mice are treated in mid-telogen but not in early telogen, suggesting that JAK inhibition cannot override the quiescence-promoting microenvironment in early telogen. Several crucial molecular events differentiate early and mid-late telogen. BMP inhibitors and Wnt agonists rise over telogen, reducing the threshold required for HF stem cell activation (9). Tgfβ2 and Fgf7/10 upregulation in the dermal papilla dampens BMP signaling in the quiescence/activation step and contributes to early anagen initiation (23, 44). As telogen progresses, the hair germ upregulates genes involved in entry into the cell cycle and signal transduction (23). Therefore, activation of HF stem cells depends on reaching an overall responsive state within the microenvironment.

Treatment with tofacitinib upregulated the expression of TGβ2, BMP6 and LEF1 in human DP spheres, providing a potential mechanism by which JAK inhibition activates the DP. That said, definitive activation of the hair cycle is likely governed by the balance of activating and inhibitory signals, and not by a single cell type or input. Therefore, it is possible that JAK inhibition of the DP (or the hair germ) is buffered by opposing signals at early stages of telogen, but as the environment becomes more permissive, the signal can induce activation.

In humans, the data suggest that JAK3 inhibition via tofacitinib treatment increases the growth rate of anagen hair shafts (skin grafts and organotypic culture assays) and enhances the inductivity of human DP spheres (neogenesis assays). Investigation of the molecular effects of tofacitinib treatment revealed that treatment may cause a molecular restoration of a subset of genes that are disrupted in culture but are present in fully inductive DP cells (FIG. 4C, 4D). This finding suggests that tofacitinib treatment can enhance applications such as autologous cell transplantation approaches for treatment of hair loss.

### Materials and Methods

Study Design.

It was hypothesized that inhibition of JAK-STAT signaling promotes entry into the hair cycle in mice. To determine the onset of anagen, backskin hairs were trimmed with an electric shaver, and entry of HF into anagen was observed by the appearance of darkening skin and by hair regrowth. For the hair cycle experiments described in FIG. 1A, biopsies were taken from 2 mice/time point, and the experiment was replicated on 3 sets of littermates. Data presented in the graph in FIG. 1B was generated by observing skin darkening over time post treatment, as shown in FIG. 5E. For the experiments described in supp. FIGS. 1C&D, 3-4 mice were used per group (7 week vs. 8.5 week), and the experiment was replicated 3 times. For the experiment described in FIGS. 1B and 5E, 4 mice were treated with control (half of the back skin) and ruxolitinib (half of the back skin) and 4 mice were treated with control (half of the back skin) and tofacitinib (half of the back skin). The experiment presented in FIG. 1E was independently replicated once, with 3 mice/condition. The experiments described in FIG. 2A, 3 mice/genotype were utilized, and the experiment was replicated in 2 sets of littermates.

Gene expression profiling was performed on whole skin biopsies from 12 B6/C57 female mice, 8.5 weeks of age. Biopsies were taken from dorsal skin at day 0 (T0) of the experiment. Mice were treated daily with DMSO, ruxolitinib and tofacitinib on day 1-4, at which point a second biopsy was taken from treated mice at day 5 (T5). Quality control was performed using the affy analysisQC package from http://arrayanalysis.org/. Two samples, DMSO1 T5 and RUXO3 T5, were removed from further downstream analysis because they failed quality control. Data were normalized using the GCRMA method as implemented in affyAnalysisQC. LIMMA was used with a multilevel model to identify genes that were differentially expressed between samples at T5 and T0 for each treatment group (DMSO, RUXO, and TOFA) using a threshold of fold change=1.5 and P<0.05. Results from the differential expression analysis were uploaded to Ingenuity Pathway Analysis (IPA) in order to identify molecular pathways that were overrepresented in each of the lists of differentially expressed genes for each of the treatment groups.

For the DP spheres microarray analysis, cells were prepared from three different donors, and Affymetrix human HG-U133 PLUS 2.0 arrays were hybridized at the CUMC genomics facility. Quality control and data normalization was performed as described above. For the organ culture experiment, 3 sets of repeats were performed, each times using HF derived from a single individual.

Mice.

All wildtype mice in this study (adult and neonatal) are of C57/B6 background, either bred in the laboratory or purchased from the Jackson Laboratory. ICR-SCID mice (IcrTac:ICR-Prkdc^{scid}) for grafting experiments were purchased from Taconic. Athymic nude mice for the hair patch assay were purchased from Charles River. Rag1−/− and Tcr β/δ−/− mice were purchased from the Jackson laboratory (stock number 002216 and 002122, respectively). All animals were maintained in an AAALAC Institute for Comparative medicine at Columbia University. Procedures were performed using institutional animal care and use committee-approved protocols.

Human Specimens.

Scalp skin for grafting experiments were obtained from Advanced Bioscience Resources (ABR) Inc. Occipital scalp follicles were from discarded tissue obtained during hair transplantation surgery, in accordance with the Declaration of Helnsinki, after an exemption was received under 45 CFR 46 by the Institutional Review Board exemption at Columbia University.

Pharmacological Inhibitors of JAK-STAT and Other Drugs Used in this Study.

Ruxolitinib (INCB018424) was purchased from ChemieTek (catalog number CT-INCB). Tofacitinib was purchased from AbMole BioScience (catalog number 477600-75-2). Hedgehog agonist (SAG) was purchased from EMD Millipore (catalog number 566660). JAK-STAT inhibitors were dissolved in DMSO and used at 2-3% for in vivo experiments, as indicated, and 400 nM for in vitro experiments. SAG was used at 120 uM, as described in Paladini et al (2).

Antibodies and Immunofluorescence.

Immunofluorescence on fresh frozen sections of mouse skin was performed as described previously (1). All fluorescence images were taken on the Zeiss LSR Excited confocal microscope. All bright field images were taken on Zeiss Axioplan 2 system. Primary antibodies used and dilutions can be found in Table 1. Nuclei were stained using 4′,6-diamidino-2-phenylindole (DAPI).

Analysis of Differential Gene Expression by qPCR Array.

Total RNA was isolated from mouse dorsal skin at indicated time points by using the RNeasy Minikit (Qiagen) in accordance with the manufacturer's instructions. Skin was harvested from 3 mice/time points, and 4 time points were tested (12 individual mice were utilized for the analysis). Total RNA (2 μg) was reverse transcribed with oligo (dT) primers and SuperScript III (Invitrogen). Resulting cDNA from each sample was aliquoted into a single JAK-STAT signaling qPCR Array (Qiagen/SABbioscience catalog number PAMM-039, gene list provided online). The array includes 84 genes related to the JAK-STAT pathway, plus five housekeeping genes and quality controls. Real-time PCR was performed on an ABI 7300 (Applied Biosystems). Data analysis was performed using RT2 Profiler PCR Array data analysis software, provided by SABioscience. Fold change in expression was determined using the ΔΔCₜ method, and values utilized in downstream analysis were derived by taking the means of fold changes in 3 biological replicates per time point.

GEDI.

Values for average log₂FC were calculated as—DDCₜ relative to post natal day 17 (early telogen) were used to perform Gene Expression Dynamic Index (GEDI) analysis in order to visualize how “metagenes” identified with a self-organizing map algorithm vary across samples. Metagenes are clusters of genes that show similar temporal expression patterns across samples (61) and that are assigned to a single pixel in a two dimensional grid. Neighboring pixels demonstrate similar expression patterns to one another. The self-organizing maps were then rendered using the level plot function in the lattice package in R.

Proliferation Experiments.

C57/B6 mice at 8.5 weeks of age were treated on half of the dorsal back with either topical vehicle control (left side) or JAK inhibitors (right side). Four hours post treatment each mouse received a single injection of 20 mg/kg 5-ethynyl-2′-deoxyuridine (EdU) (Invitrogen). One hour after injection, skin was harvested, fixed and stained using the Click-iT EdU Alexa Fluor 488 Imaging Kit (Life Technologies) per manufacturer's instructions, and co-stained for P-cadherin. Experiment was replicated once, with 1 mouse/time point.

Microarray Analysis

Total RNA was extracted using the Qiagen RNAeasy micro kit. The Ovation RNA Amplification kit (Nugen) was used to generate amplified cDNA for microarray analysis. Arrays used were Affymetrix mouse 430 2.0, hybridized at the CUMC microarray facility. qPCR analysis was performed on the ABI 7300 cycler. Primers sequences available in Table 2.

For the DP spheres microarray analysis, total RNA was extracted from DP spheres cultured in DMSO, ruxolitinib or tofacitinib from each of the three donors using the Qiagen RNAeasy micro kit. The Ovation RNA Amplification kit (Nugen) was used to generate amplified cDNA for microarray analysis. LIMMA was used with a linear model treating both treatment and the blocking factor, donor, as fixed effects. Contrasts of interest between treatments were used to identify genes that were differentially expressed between pairs of treatments for the treatments DMSO, RUXO, and TOFA. A threshold of absolute fold change=1.5 and P<0.05 was employed. For GSEA, the HG_U133_Plus_2.0 chip annotation file downloaded from ftp.broad.mit.edullpub/gsedannotations/was used to annotate the Affymetrix ProbeSets and to remove duplicate ProbeSets for a given Gene Symbol. Duplicates were removed based on the maximum absolute fold-change observed for each Gene Symbol. Pre-ranked gene lists were generated for each treatment contrast of interest, using the moderated t-statistics returned by LIMMA. GSEA was performed on these lists to identify KEGG pathways overrepresented in each list (ES p-value 0.01).

Gene Set Enrichment Analysis

The gene set enrichment analysis comparing tofacitinib-treated and control cells was performed by generating a list of all detected transcripts ranked by differential expression (p-value), and direction of fold change (up or down). This ranked list was tested for enrichment of the union of territories 2 and 4 (gene list provided in Table 3) as described previously (35). A null distribution for computing the normalized enrichment score (NES) and its associated p-value was obtained by label shuffling to randomize the gene rankings. These randomized sets were then used to compute null enrichment scores over 5000 iterations to generate a null distribution. The observed leading edge enrichment score was normalized to this distribution and a 2-tailed p-value was generated for this NES.

Human Hair Follicle Organ Culture Assay.

Adult human follicles were microdissected from occipital skin under sterile conditions as previously described.

Individual follicles were placed in a 24-well tissue culture plate in Williams E medium supplemented with hydrocortisone, Insulin and Glutamine (62), in the presence of DMSO, ruxolitinib or tofacitinib (400 nM). Media was replaced every 2 days, and images of individual follicles were taken every 2 days. Analysis of the growth rate of each follicle was performed using Image J.

Patch Assay with Dermal Papilla Spheres.

DP cells were grown in spheres as previously described (35). DMSO, ruxolitinib or tofacitinib were added to the medium (400 nM). Each hanging drop contained 1000 cells, which aggregated to form DP spheroids after 24 hours. 48 hours after plating, 500 spheres in each condition were collected for use in the patch assay. Each experiment was performed with cells derived from a single individual, and four separate experiments were conducted. Keratinocytes were isolated from new born mice following the protocol outlined by Lichti et al (63). Cells were cultured in dkSFM for 2-4 days prior to being harvested for the patch assay. 1×10⁶ newborn murine keratinocytes were then mixed together with 500 human DP spheres in each condition, and injected subdermally into dorsal skin of a nude mouse. 12 days after performing the injections, cysts had developed in the dermis, some which contained HF and hair fibers. These cysts were collected, photographed and then digested in 0.35% collagenase. The digested slurries were spread onto microscope slides and hair fiber counts were manually performed under a stereo microscope.

Human Skin Grafting Assay.

Human embryonic scalp skin (16 weeks old), approximately 2×2 cm in diameter, was grafted onto the back of SCID mice. The mice were bandaged, and the grafts were left to recover for 6 weeks. After 6 weeks small hairs could be observed on the grafts. Grafts were treated daily with topical application of vehicle control, ruxolitinib or tofacitinib. Treatment continued for 4 weeks, and pictures were taken every 3-5 days. Experiments were performed with skin from 3 separate donors.

Quantification of hair growth was performed using ImageJ. Taking advantage of the fact that the donor hair was dark and the grafted mouse was white, the intensity of pigmentation was measured as a proxy for density of HF. Since control and experimental treatments were performed on the same graft, each image allows for the direct comparison between vehicle- and drug-treated skin. Intensity was scored across 3 lines/image and averaged to generate the histograms shown in FIGS. 3A and 8A. Darker regions are given a lower value than bright regions. Ratio was calculated by averaging the intensity values for the control treated side, by the intensity values for drug-treated side, accounting for pre-treatment differences in hair growth on the graft and for changes in hair density occurring over time.

Statistical Analysis.

A significance level of p=0.05 was used for all tests. For both the organ culture longitudinal study (FIGS. 3 B& C) and the skin pigmentation study (FIG. 1B) nonparametric longitudinal data analysis was performed using the R package nparLD to test the hypothesis that there exists a treatment by time interaction, i.e., time profiles are not parallel, for comparisons of pairs of treatments. In the organ culture study an F1-LD-F1 design was employed. In the skin pigmentation study, an LD-F2 model was performed for the Ruxo vs Control and Tofa vs Control comparisons to account for the paired design and an F1-LD-F1 model was used for the Tofa vs Ruxo comparison because the data were unpaired. The ANOVA-Type statistic was tested for significance at alpha=0.05 (a randomized complete block design was used to analyze the human dermal papillae spheres patch assay (FIG. 3E). Three treatments each were applied to DP spheres from each of three donors. Donor was treated as a fixed blocking factor. The unit of statistical analysis is the number of observed hair follicles. The data were analyzed using a linear mixed effects model treating the blocking factor, Donor, as a random effect. The R packages 1me4 and 1merTest were used to test whether the fixed factor, Treatment, contributed significantly to the number of follicles observed and to perform post-hoc comparisons of treatment means. The package 1merTest employed the Satterthwaite approximation in order to obtain p-values and denominator degrees of freedom

### REFERENCES

1. L. Xing et al., Nat Med 20, 1043 (September, 2014).
2. R. D. Paladini, J. Saleh, C. Qian, G. X. Xu, L. L. Rubin, J Invest Dermatol 125, 638 (October, 2005).
3. M. Cutolo, Ther Adv Musculoskelet Dis 5, 3 (February, 2013).
4. T. Zhou et al., Leukemia, (Jul. 4, 2013).
5. R. A. Mesa, U. Yasothan, P. Kirkpatrick, Nat Rev Drug Discov 11, 103 (February, 2012).
6. D. C. Borie, J. J. O'Shea, P. S. Changelian, Trends Mol Med 10, 532 (November, 2004).
7. S. Verstovsek et al., N Engl J Med 366, 799 (Mar. 1, 2012).
8. S. Muller-Rover et al., J Invest Dermatol 117, 3 (July, 2001).
9. M. V. Plikus et al., Nature 451, 340 (Jan. 17, 2008).
10. M. V. Plikus et al., Science 332, 586 (Apr. 29, 2011).
11. L. Alonso, E. Fuchs, J Cell Sci 119, 391 (Feb. 1, 2006).
12. R. Schmidt-Ullrich et al., Development 133, 1045 (March, 2006).
13. I. Brownell, E. Guevara, C. B. Bal, C. A. Loomis, A. L. Joyner, Cell Stem Cell 8, 552 (May 6, 2011).
14. W. M. Woo, H. H. Zhen, A. E. Oro, Genes Dev 26, 1235 (Jun. 1, 2012).
15. Y. Ito et al., J Invest Dermatol 127, 1052 (May, 2007).
16. Y. C. Hsu, L. Li, E. Fuchs, Cell 157, 935 (May 8, 2014).
17. P. Rabbani et al., Cell 145, 941 (Jun. 10, 2011).
18. R. M. Castilho, C. H. Squarize, L. A. Chodosh, B. O. Williams, J. S. Gutkind, Cell Stem Cell 5, 279 (Sep. 4, 2009).
19. A. J. Kellenberger, M. Tauchi, Exp Dermatol 22, 77 (January, 2013).
20. R. Schmidt-Ullrich et al., Development 128, 3843 (October, 2001).
21. S. Sano et al., EMBO J 18, 4657 (Sep. 1, 1999).
22. S. Sano et al., Proc Natl Acad Sci USA 97, 13824 (Dec. 5, 2000).
23. V. Greco et al., Cell Stem Cell 4, 155 (Feb. 6, 2009).
24. A. Ferrajoli, S. Faderl, F. Ravandi, Z. Estrov, Curr Cancer Drug Targets 6, 671 (December, 2006).
25. D. Gay et al., Nat Med 19, 916 (July, 2013).
26. J. E. Kloepper, K. Kawai, M. Bertolini, T. Kanekura, R. Paus, J Invest Dermatol 133, 1666 (June, 2013).
27. D. Castellana, R. Paus, M. Perez-Moreno, PLoS Biol 12, e1002002 (December).
28. A. Heine et al., Blood 122, 1192 (August 15).
29. K. K. Lin, D. Chudova, G. W. Hatfield, P. Smyth, B. Andersen, Proc Natl

Acad Sci USA 101, 15955 (Nov. 9, 2004).
30. K. Turksen, T. Kupper, L. Degenstein, I. Williams, E. Fuchs, Proc Natl Acad Sci USA 89, 5068 (Jun. 1, 1992).
31. M. H. Kwack, J. S. Ahn, M. K. Kim, J. C. Kim, Y. K. Sung, J Invest Dermatol 132, 43 (January, 2012).
32. M. Yu et al., Exp Dermatol 17, 12 (January, 2008).
33. C. Vidon et al., Joint Bone Spine 81, 100 (January, 2014).
34. K. S. Stenn, R. Paus, Physiol Rev 81, 449 (January, 2001).
35. C. A. Higgins, J. C. Chen, J. E. Cerise, C. A. Jahoda, A. M. Christiano, Proc Natl Acad Sci USA 110, 19679 (Dec. 3, 2013).
36. Y. Zheng et al., J Invest Dermatol 124, 867 (May, 2005).
37. R. R. Driskell, A. Giangreco, K. B. Jensen, K. W. Mulder, F. M. Watt, Development 136, 2815 (August, 2009).
38. M. Kawano et al., J Invest Dermatol 124, 877 (May, 2005).
39. H. Kamp, C. C. Geilen, C. Sommer, U. Blume-Peytavi, Exp Dermatol 12, 662 (October, 2003).
40. M. Rendl, L. Lewis, E. Fuchs, PLoS Biol 3, e331 (November, 2005).
41. M. Rendl, L. Polak, E. Fuchs, Genes Dev 22, 543 (Feb. 15, 2008).
42. R. Paus, K. Foitzik, P. Welker, S. Bulfone-Paus, S. Eichmuller, J Invest Dermatol 109, 518 (October, 1997).
43. K. Foitzik, R. Paus, T. Doetschman, G. P. Dotto, Dev Biol 212, 278 (Aug. 15, 1999).
44. N. Oshimori, E. Fuchs, Cell Stem Cell 10, 63 (Jan. 6, 2012).
45. P. Zhou, C. Byrne, J. Jacobs, E. Fuchs, Genes Dev 9, 700 (Mar. 15, 1995).
46. K. Kratochwil, M. Dull, I. Farinas, J. Galceran, R. Grosschedl, Genes Dev 10, 1382 (Jun. 1, 1996).
47. B. Hu et al., Genes Dev 24, 1519 (Jul. 15, 2010).
48. C. A. Ambler, F. M. Watt, Dev Dyn 236, 1595 (June, 2007).
49. A. Subramanian et al., Proc Natl Acad Sci USA 102, 15545 (Oct. 25, 2005).
50. J. Doles, M. Storer, L. Cozzuto, G. Roma, W. M. Keyes, Genes Dev 26, 2144 (Oct. 1, 2012).
51. J. Goldstein et al., Genes Dev 28, 983 (May 1, 2014).
52. Z. Wang, G. Li, W. Tse, K. D. Bunting, Blood 113, 4856 (May 14, 2009).
53. F. D. Price et al., Nat Med 20, 1174 (October).
54. M. T. Tierney et al., Nat Med 20, 1182 (October, 2014).
55. S. J. Divito, T. S. Kupper, Nat Med 20, 989 (September).
56. E. Festa et al., Cell 146, 761 (Sep. 2, 2011).
57. C. A. Jahoda, A. M. Christiano, Cell 146, 678 (Sep. 2, 2011).
58. A. Quintas-Cardama et al., Blood 115, 3109 (April 15).
59. H. L. Geyer, R. A. Mesa, Blood 124, 3529 (December 4).
60. K. Ghoreschi et al., J Immunol 186, 4234 (April 1).
61. G. S. Eichler, S. Huang, D. E. Ingber, Bioinformatics 19, 2321 (Nov. 22, 2003).
62. D. J. Tobin, Methods Mol Biol 695, 213 (2010).
63. U. Lichti, J. Anders, S. H. Yuspa, Nat Protoc 3, 799 (2008).

### 6.2 Example 2: Oncostatin M Promotes Quiescence of Hair Follicle Stem Cells Via JAK-STAT Signaling

### Introduction

JAK-STAT signaling is involved in maintaining the quiescence of hair follicle stem cells (HFSCs) during telogen (resting phase) of the murine hair cycle. It has been demonstrated that JAK inhibition is able to initiate anagen in wild-type mouse telogen skin (1). Using gene expression and cellular dynamics analyses, it has bee shown that this process recapitulated the early events of the native hair cycle, whereby the first signs of proliferation were observed in the secondary hair germ adjacent to the dermal papilla. JAK-STAT signaling is suggested to be intricately involved with cytokine signaling. In particular, the IL-6 family of cytokines have been shown to antagonize anagen and promote catagen/telogen in mouse skin. Of the IL-6 family of cytokines, oncostatin M (OSM) has been implicated in the maintenance of quiescence of stem cells in other systems, namely in muscle stem cells. Using immunofluorescence studies, the inventors found coexpression of OSM and its receptor (OSM-Rβ) in the secondary hair germ of the murine hair follicle, in a pattern complementary to activated (phosphorylated) STAT3 and STAT5 signaling molecules, suggesting an autocrine role for OSM in the maintenance of quiescence in this compartment. These observations have been confirmed with in vitro experiments on cultured mouse keratinocytes. Using temporal and spatial conditional knock-outs of JAK-STAT signaling components, along with OSM and OSM-Rβ, the inventors tested whether OSM and its receptor are necessary and sufficient for quiescence of HFSCs in the secondary hair germ, and possibly other stem cells of the HF. The surprising role of active JAK-STAT signaling in promoting quiescence in HFSCs place it alongside pathways such as BMP signaling and Wnt inhibition in the maintenance of telogen.

### Results

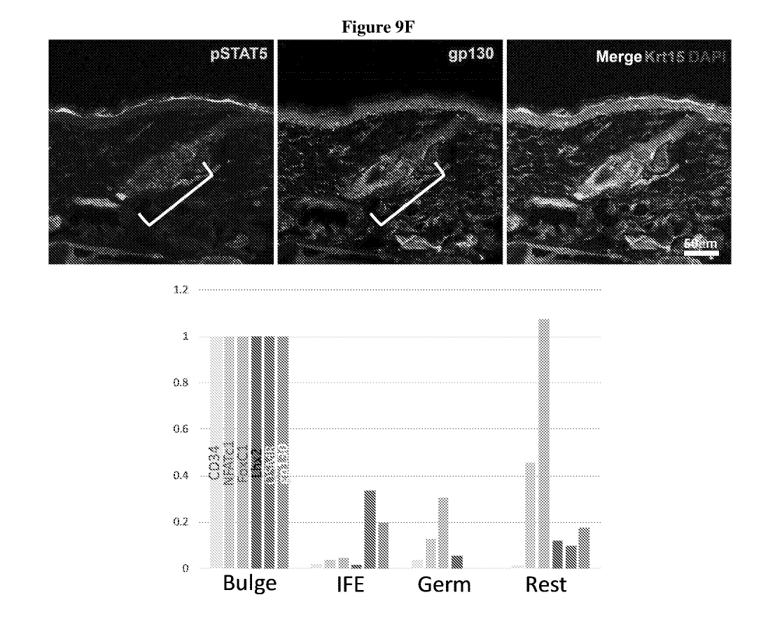

Phosphorylated STAT3 and STAT5 proteins were detected in the HFSC compartment during early and mid telogen (P46, P60), and this decreased during late telogen (P80) (FIG. 9A). OSM immunofluorescence was co-localized to the HFSC during this time. OSM receptor (OSM-Rβ) was also found to co-localize to the HFSC bulge compartment with immunohistochemistry (FIG. 9B). All images were taken at ×20 magnification. In vitro cultured keratinocytes, OSM activated STAT1, STAT3 and STAT5 transcription factors within 10 minutes of OSM treatment, and this was efficiently abrogated by Tofacitinib (TOFA) (FIG. 9C). In vivo whole skin treated with TOFA also demonstrates effective inhibition of STAT3 and STAT5 activation (FIG. 9D). Neutralizing antibodies to OSM-Rβ injected subcutaneously daily into P60 telogen skin induced a local anagen, suggesting that OSM-Rβ activation in the HFSC compartment was necessary to maintain quiescence during telogen (FIG. 9E). PBS control and neutralizing antibodies to IL-6 and its receptor had no effect (FIG. 9E). FIG. 9F shows that gp130 was also enriched in the bulge.

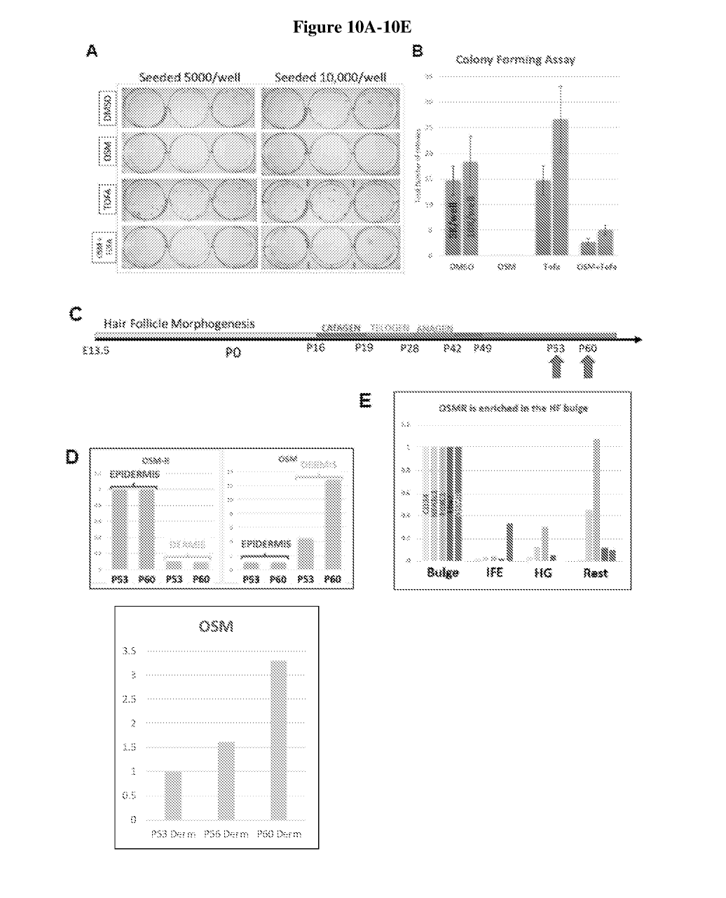

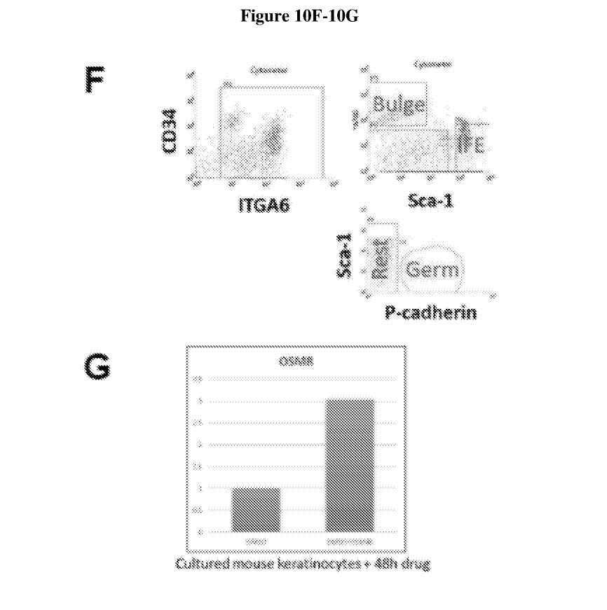

Colony forming assay with mouse keratinocytes harvested at telogen showed that OSM have a growth inhibitory effect on keratinocytes, which was reversed partially with the addition of Tofacitinib (FIG. 10A). Tofacitinib alone was also noted to increase the number of keratinocyte colonies (FIG. 10B), consistent with previous findings (4). Skin harvested in the second telogen at 7.5 and 8.5 weeks (P56 and P60) were dissociated and the epidermis and dermis were analyzed by qRT-PCR. D OSM-Rβ was preferentially expressed by the epidermis, while OSM was produced in the dermis (FIG. 10C). OSM production also increased between P53 and P60. In the epidermis, OSM-Rβ was enriched in the bulge compartment, further suggesting that OSM signaling was maintaining quiescence of this population during telogen (FIG. 10E). FIG. 10F shows cell sorting strategy for mouse epidermis to isolate CD34+ITGA6+ HFSCs, adapted from (5). In cultured mouse keratinocytes, treatment with recombinant mouse OSM at 20 ng/ml upregulated the expression of OSM-Rβ (FIG. 10G). This suggests that a positive-feedback mechanism maintained quiescence of HFSC.

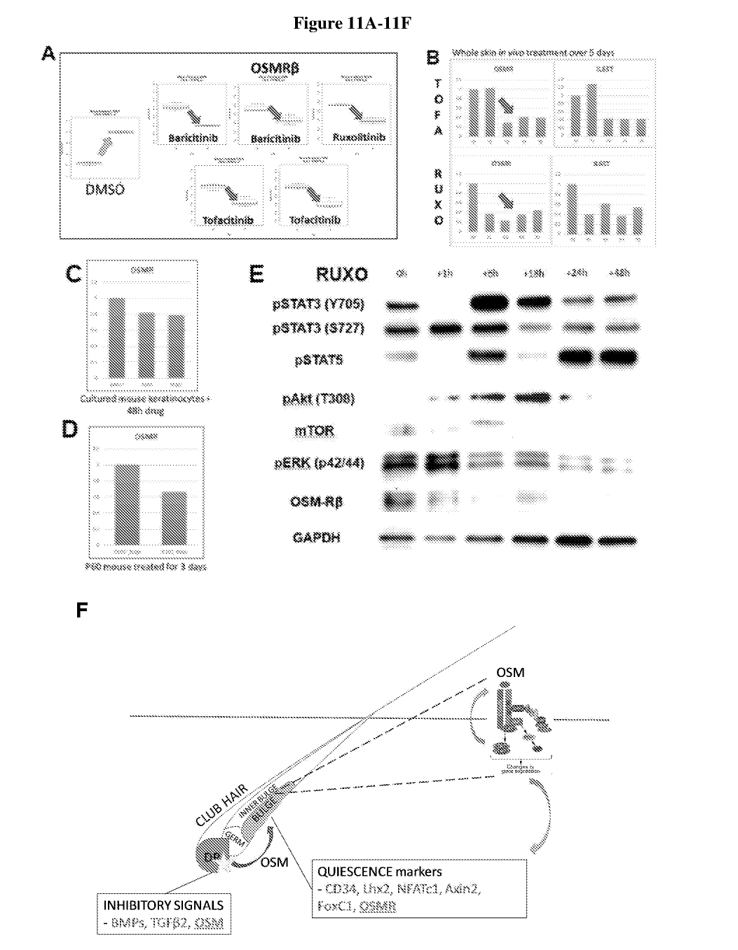

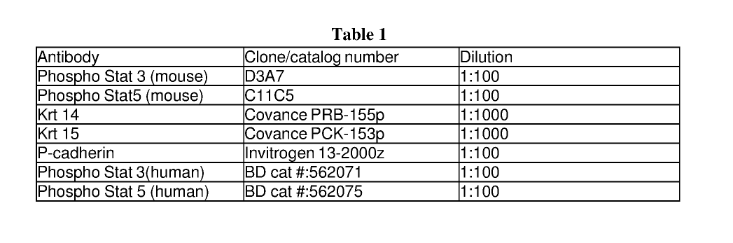

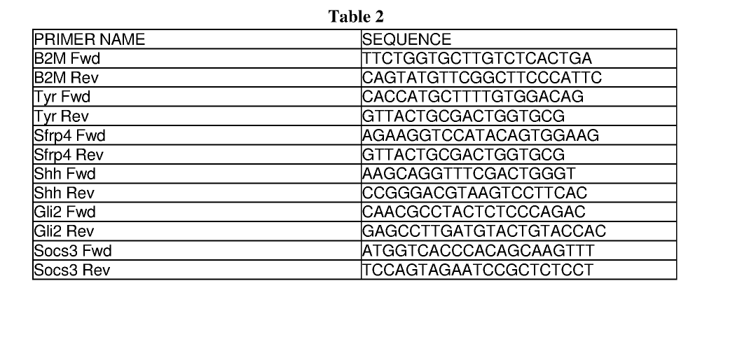

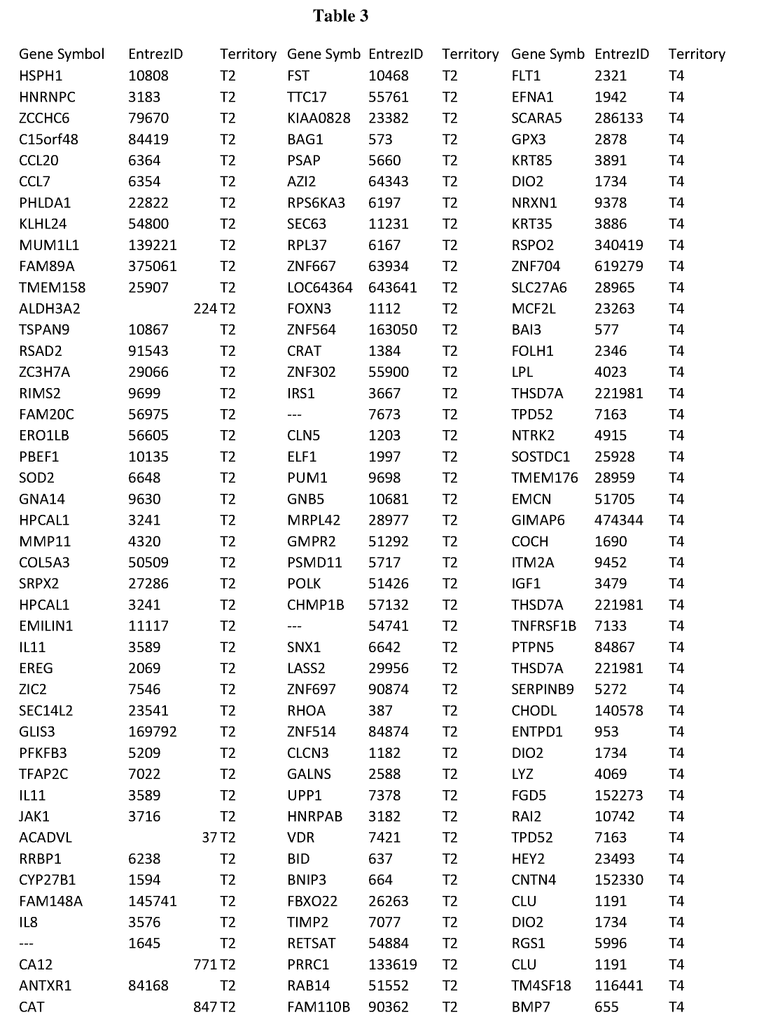

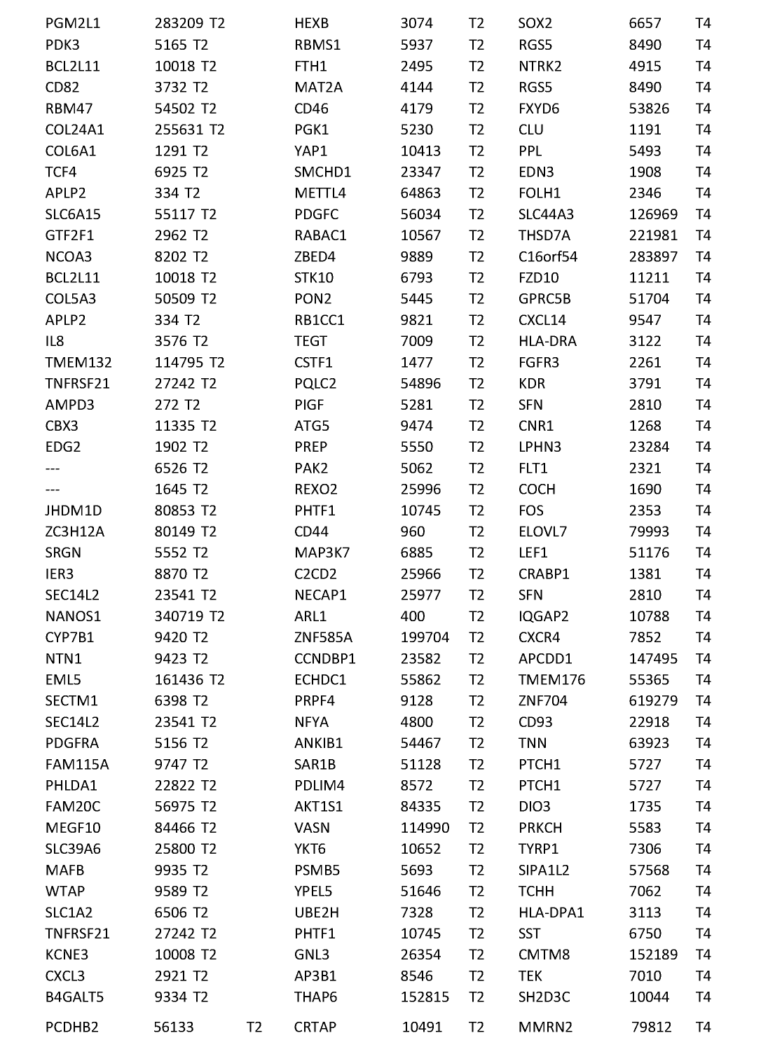

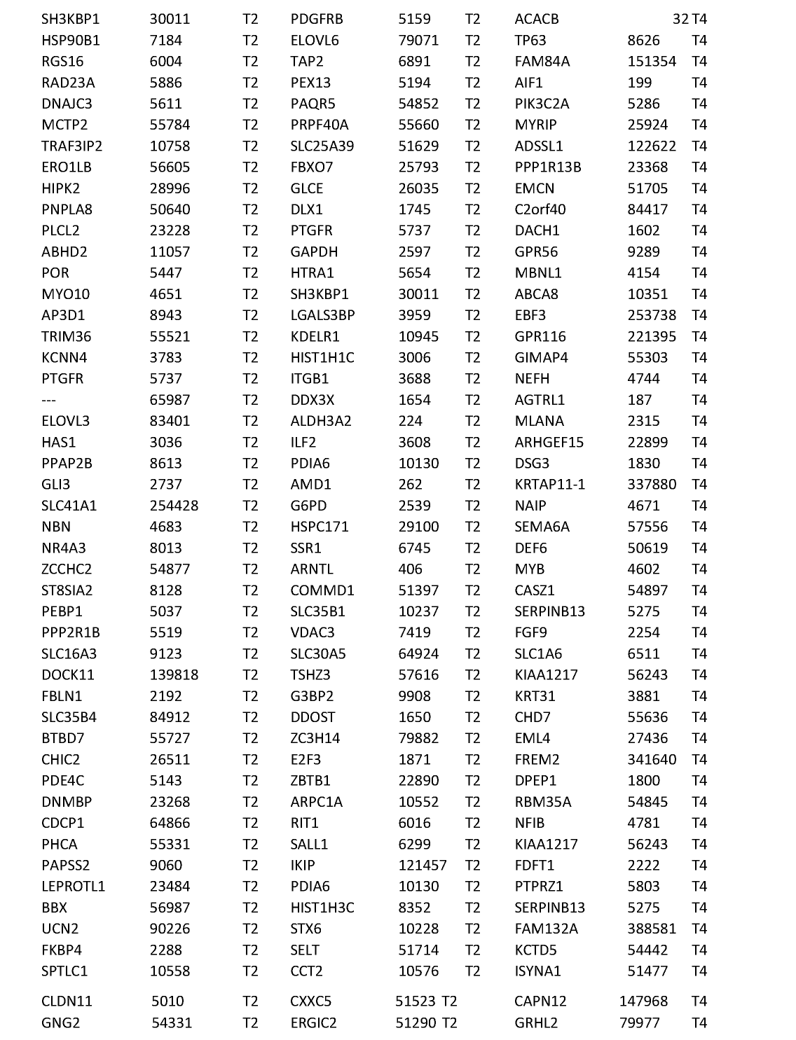

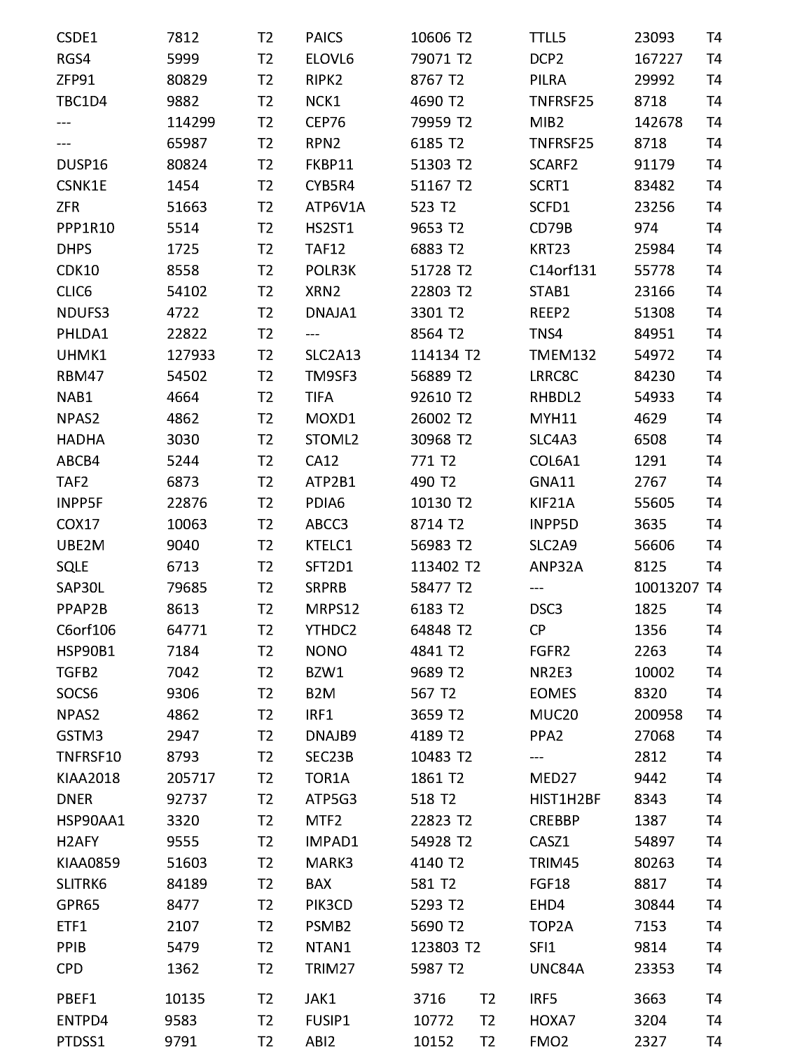

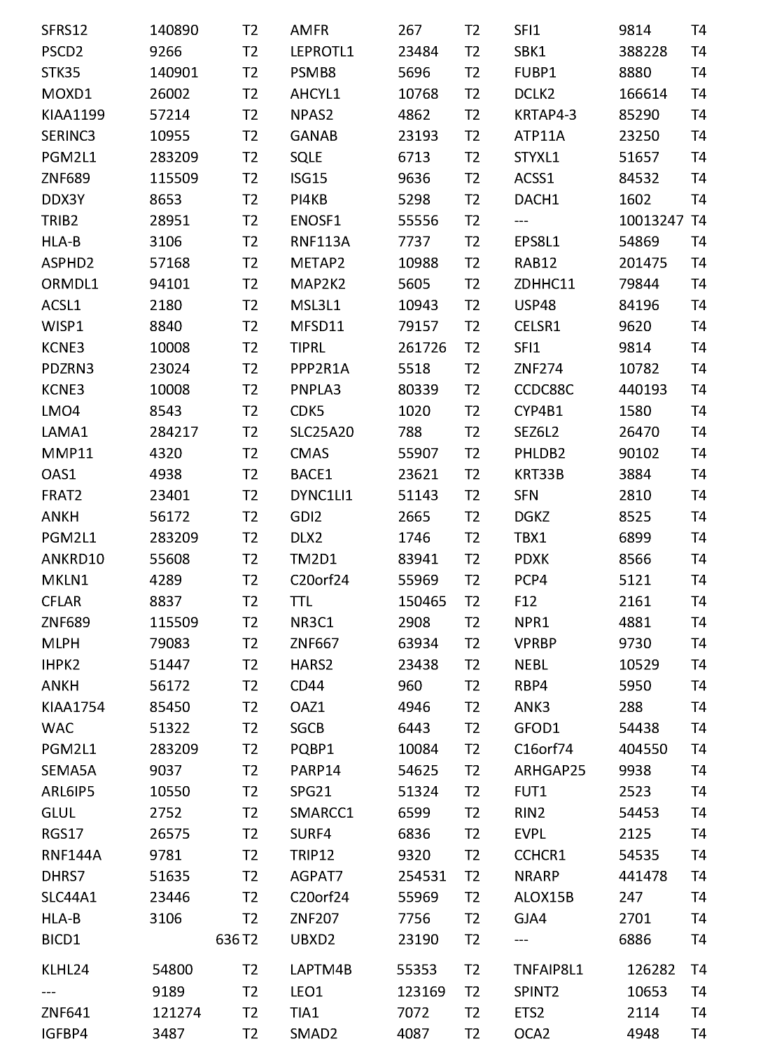

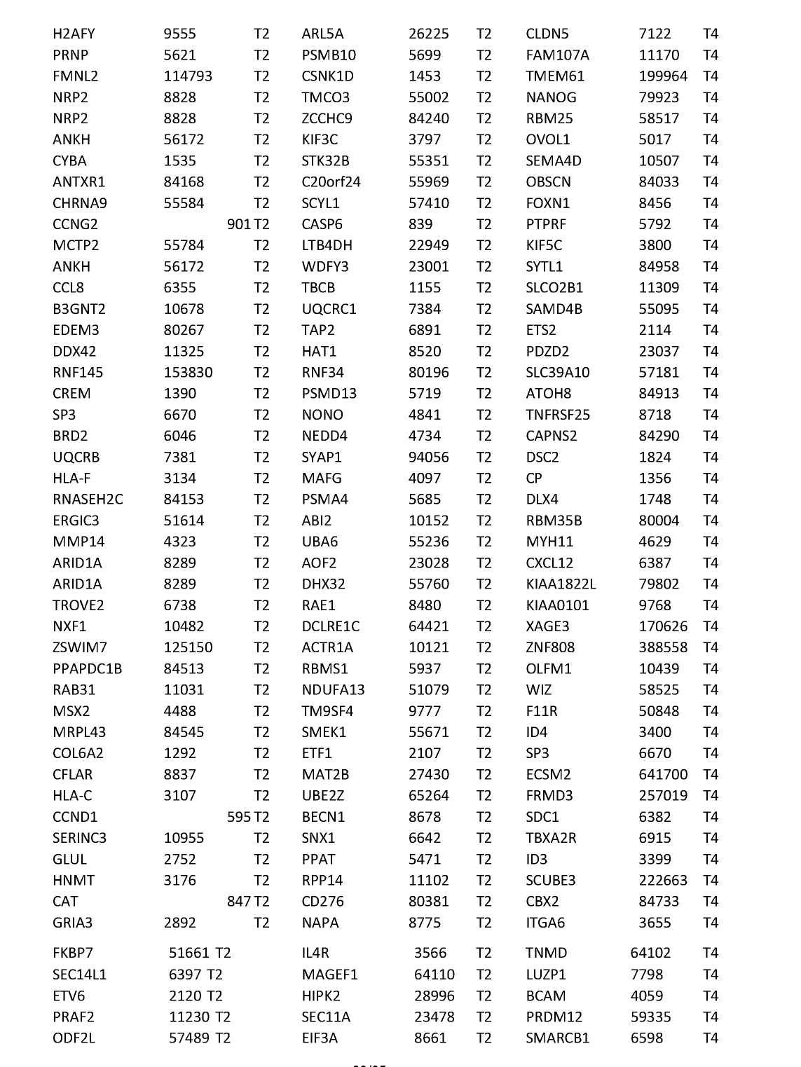

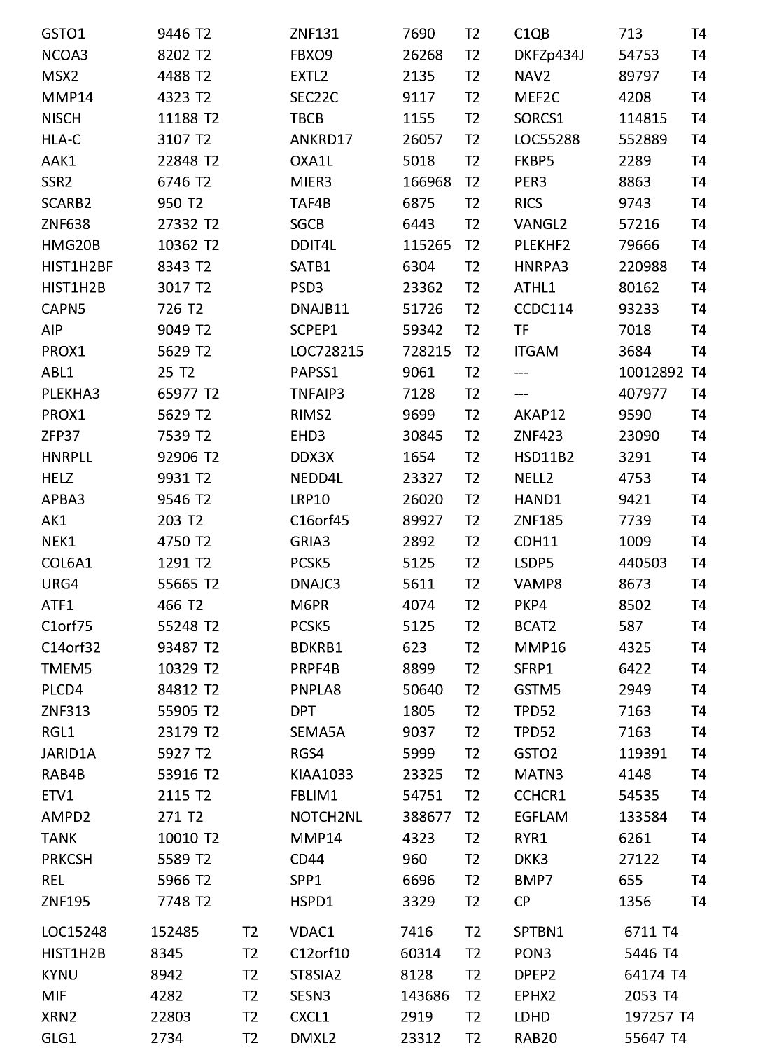

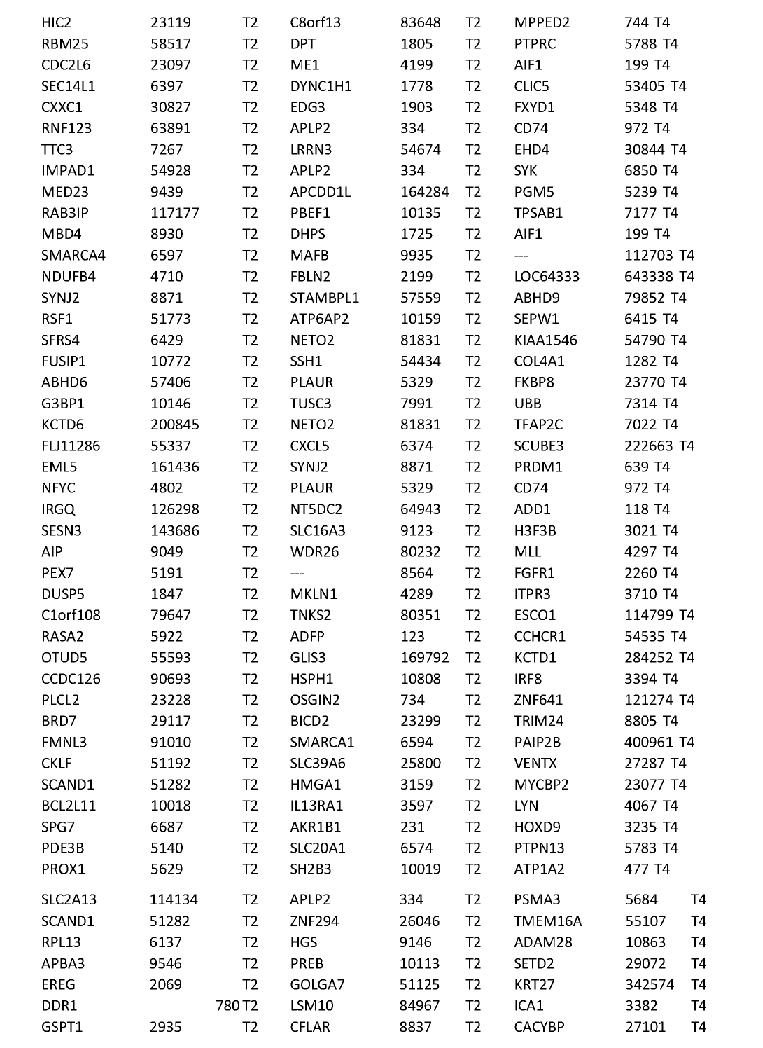

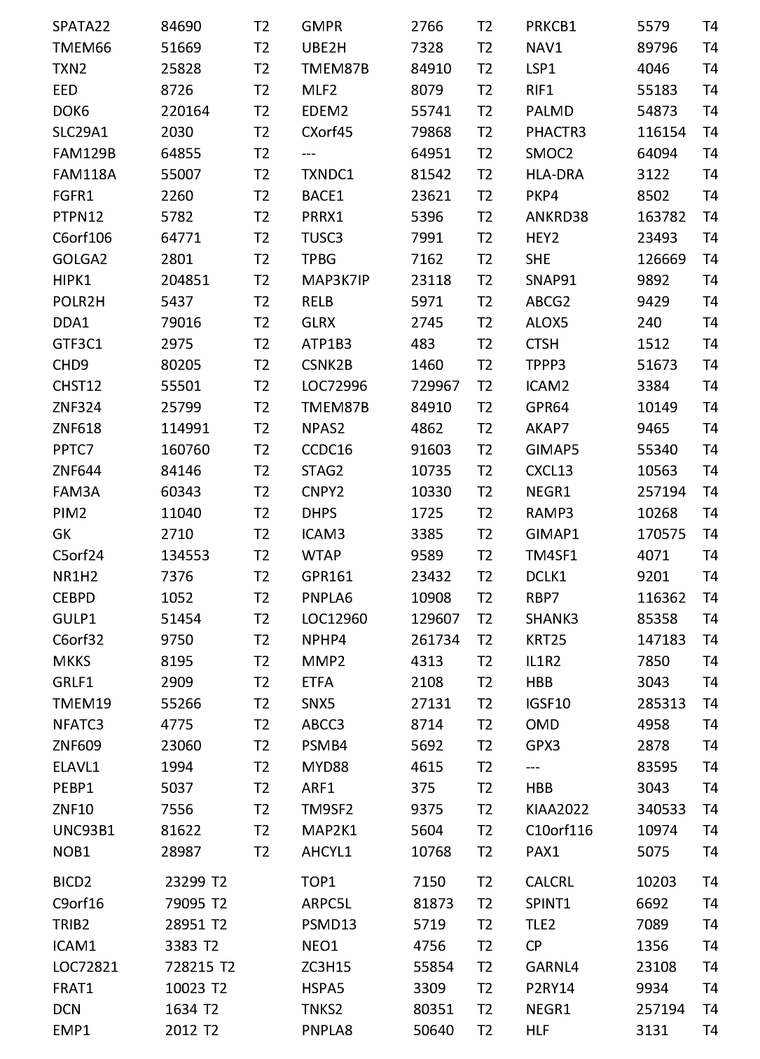

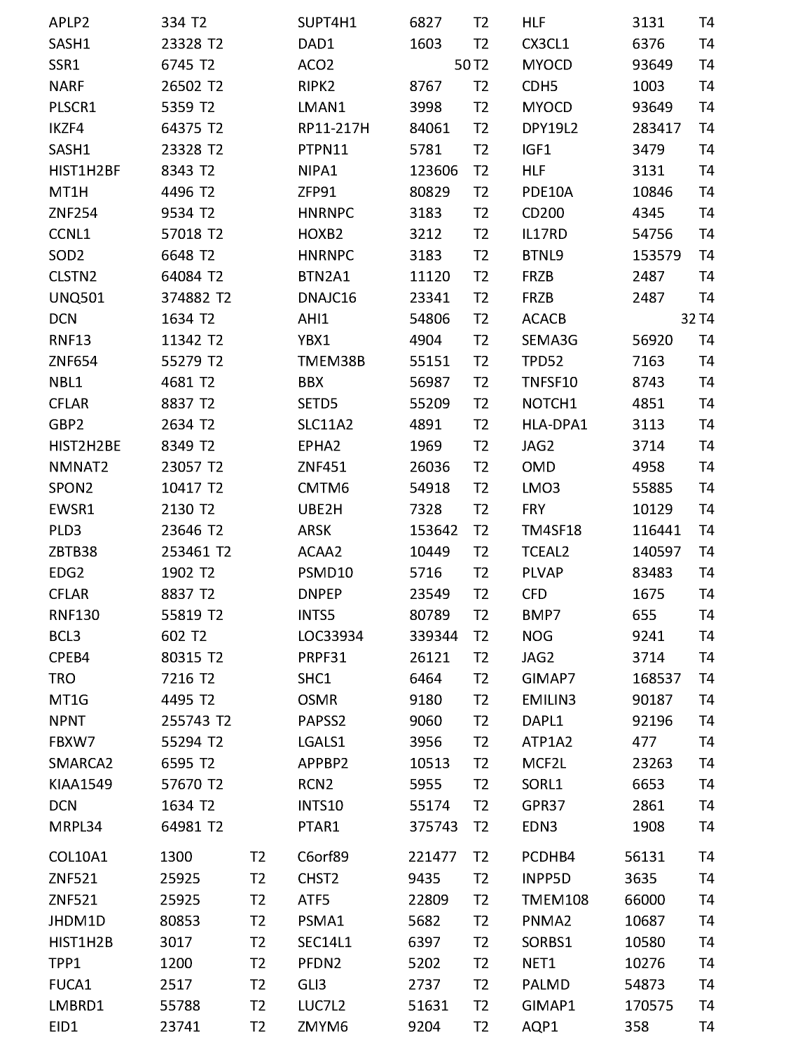

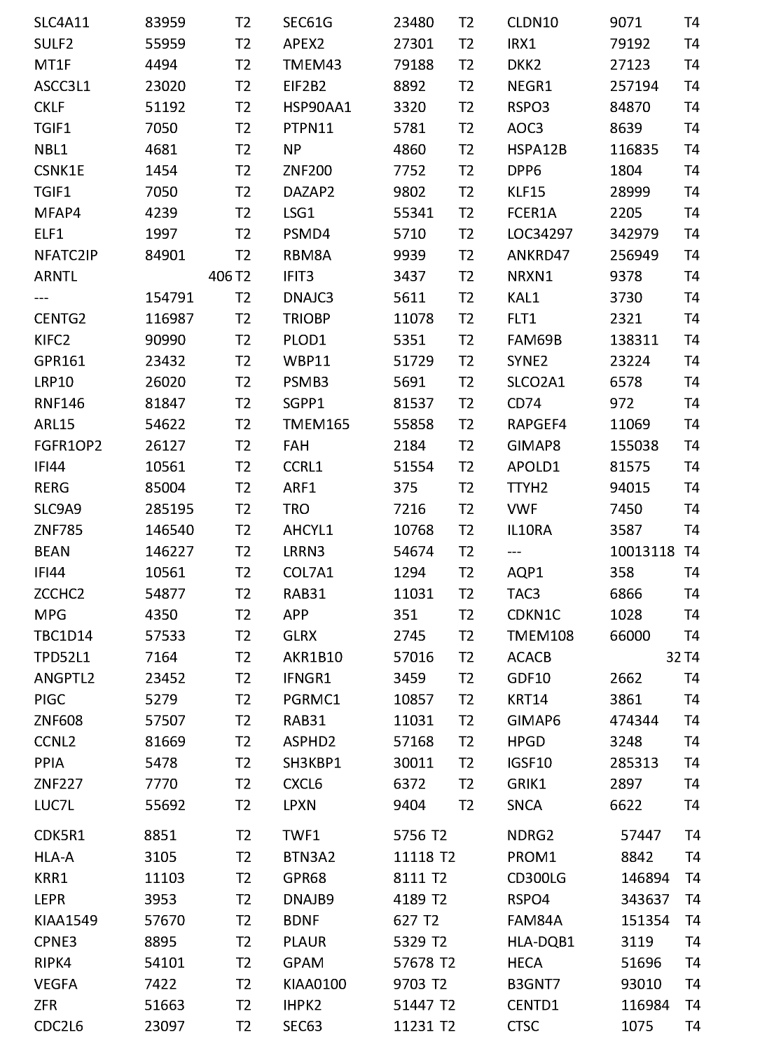

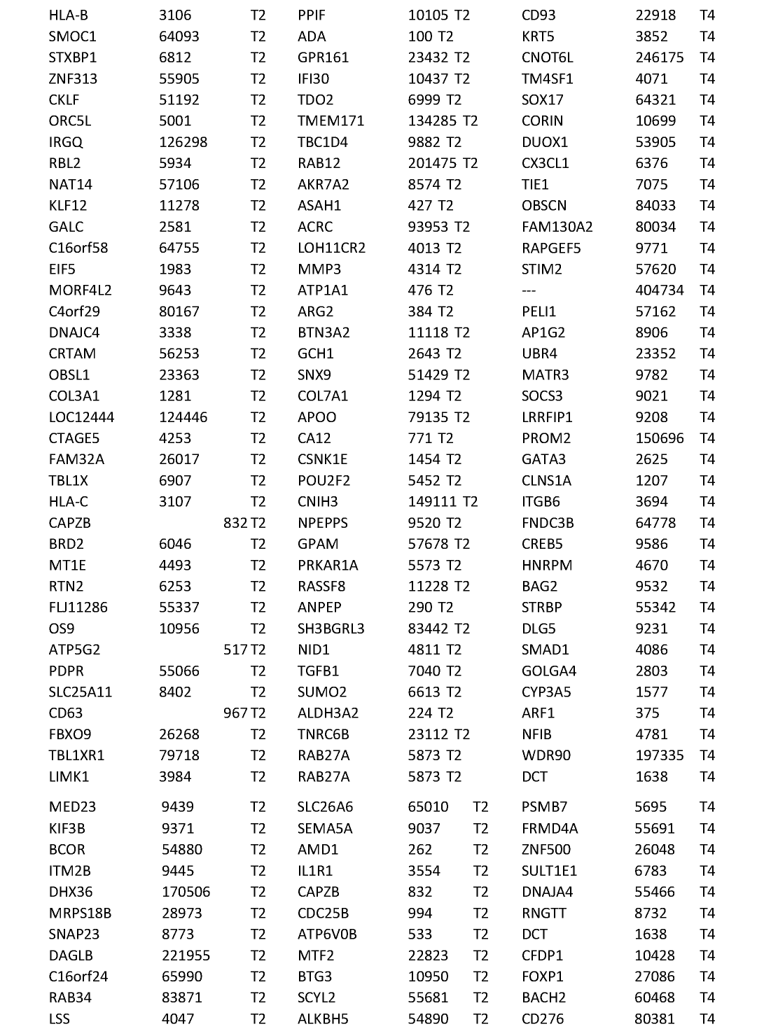

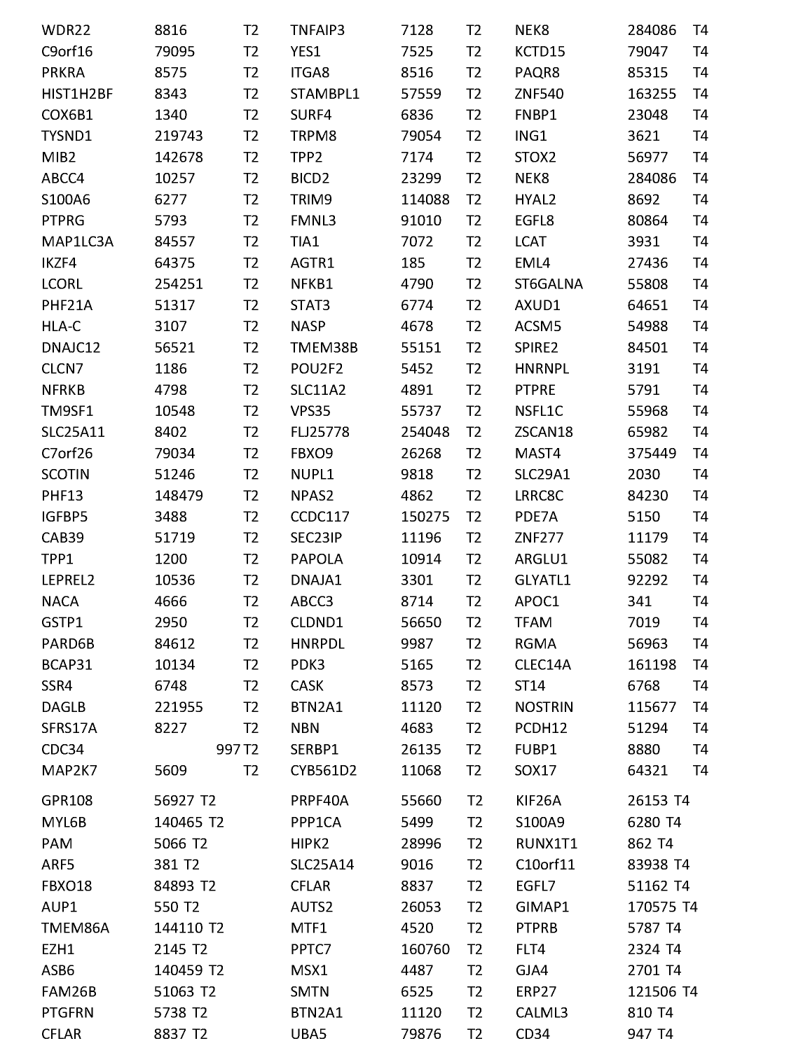

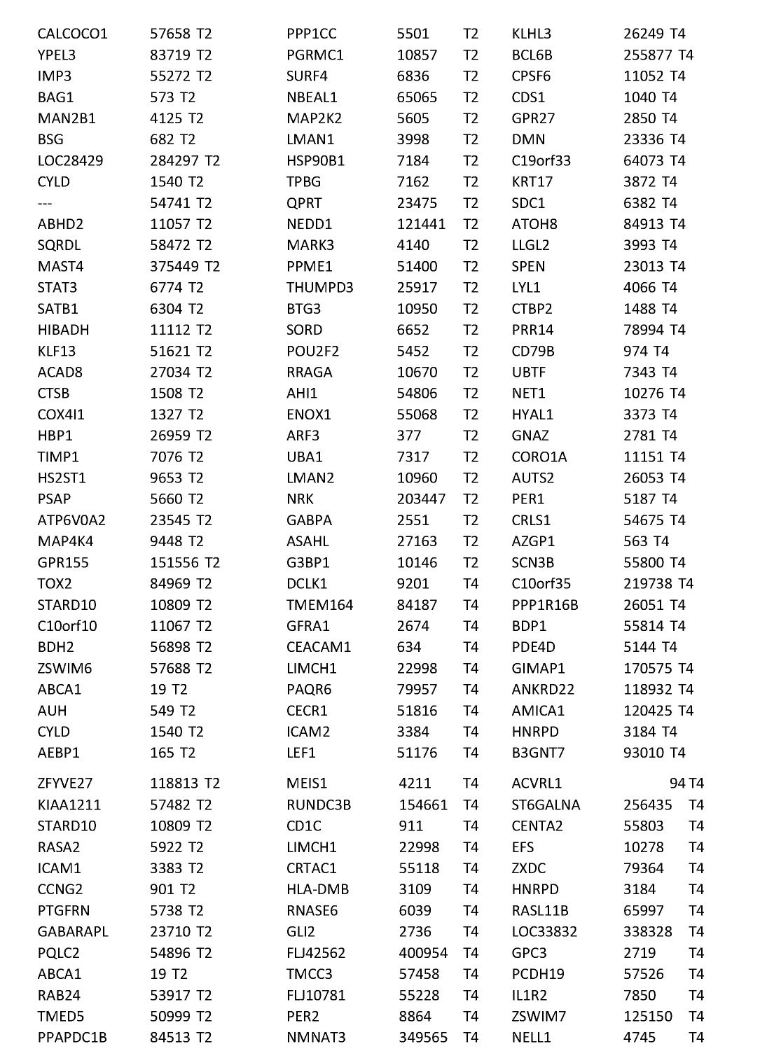

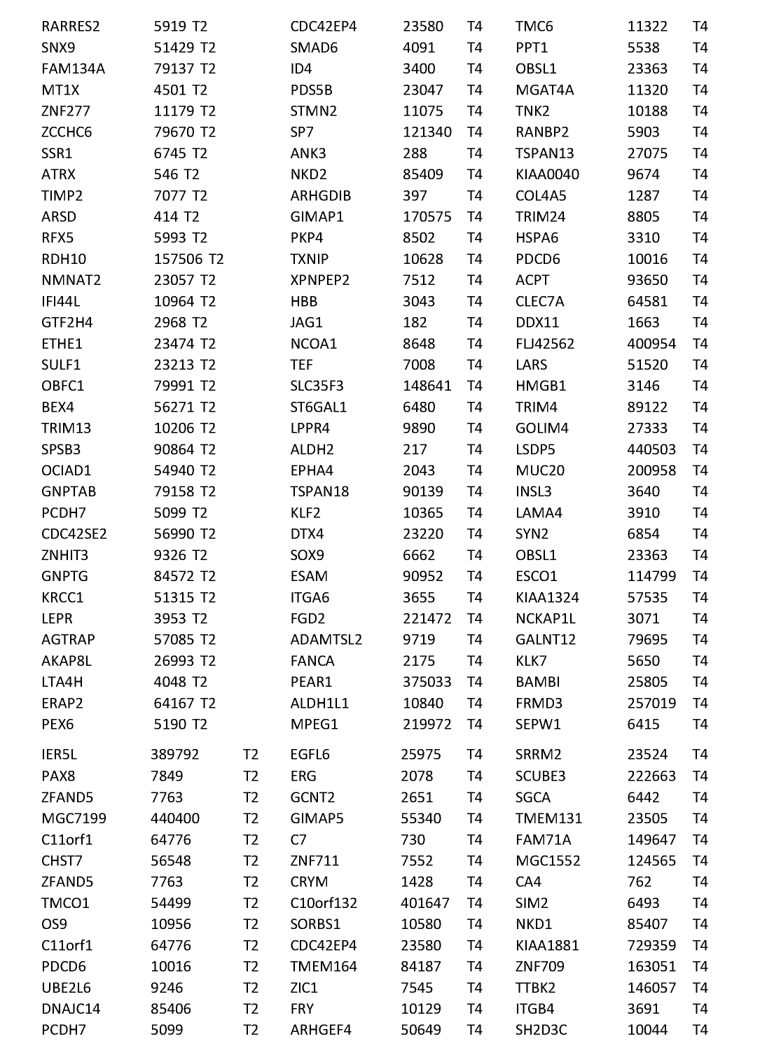

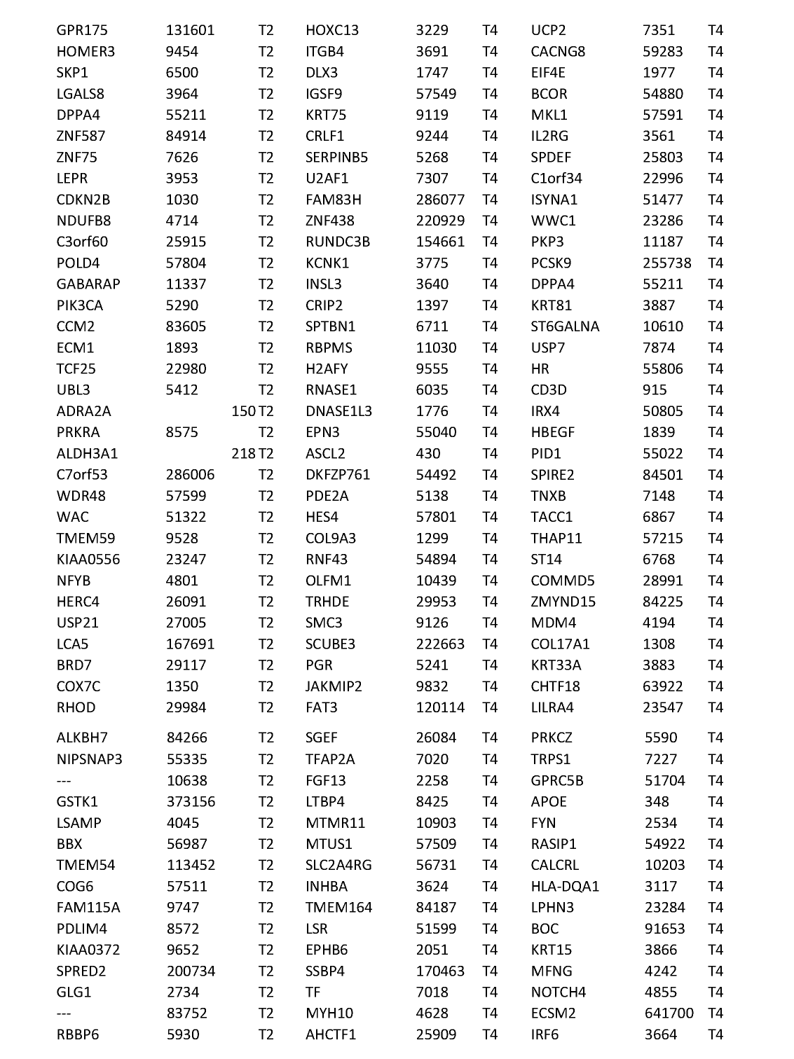

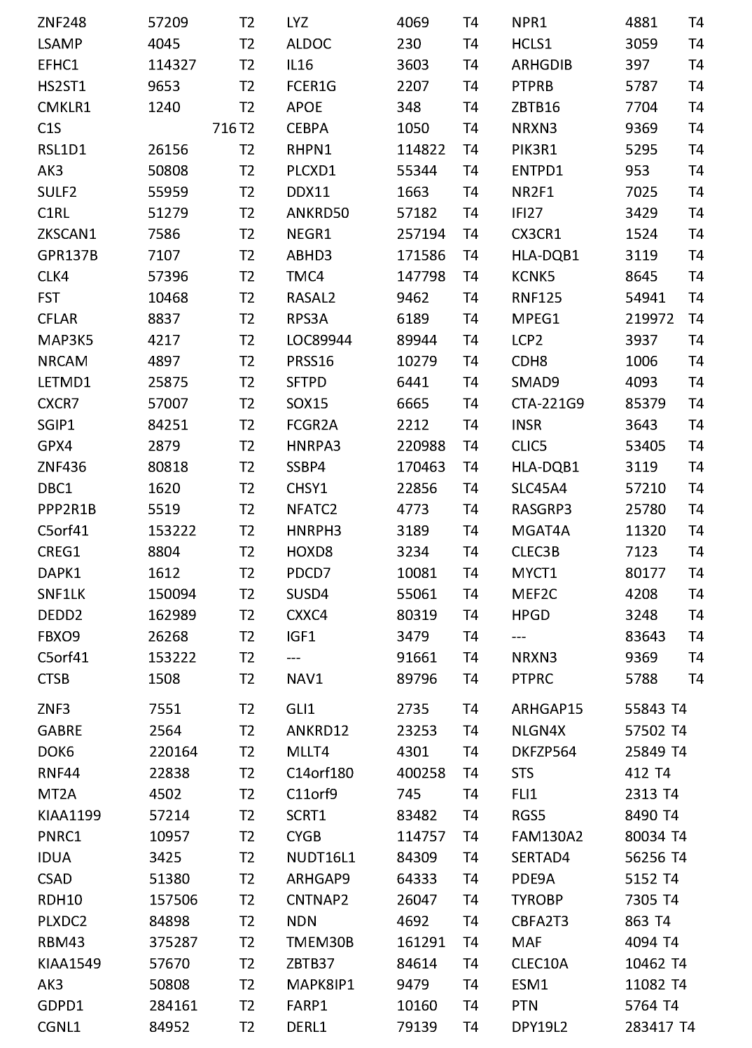

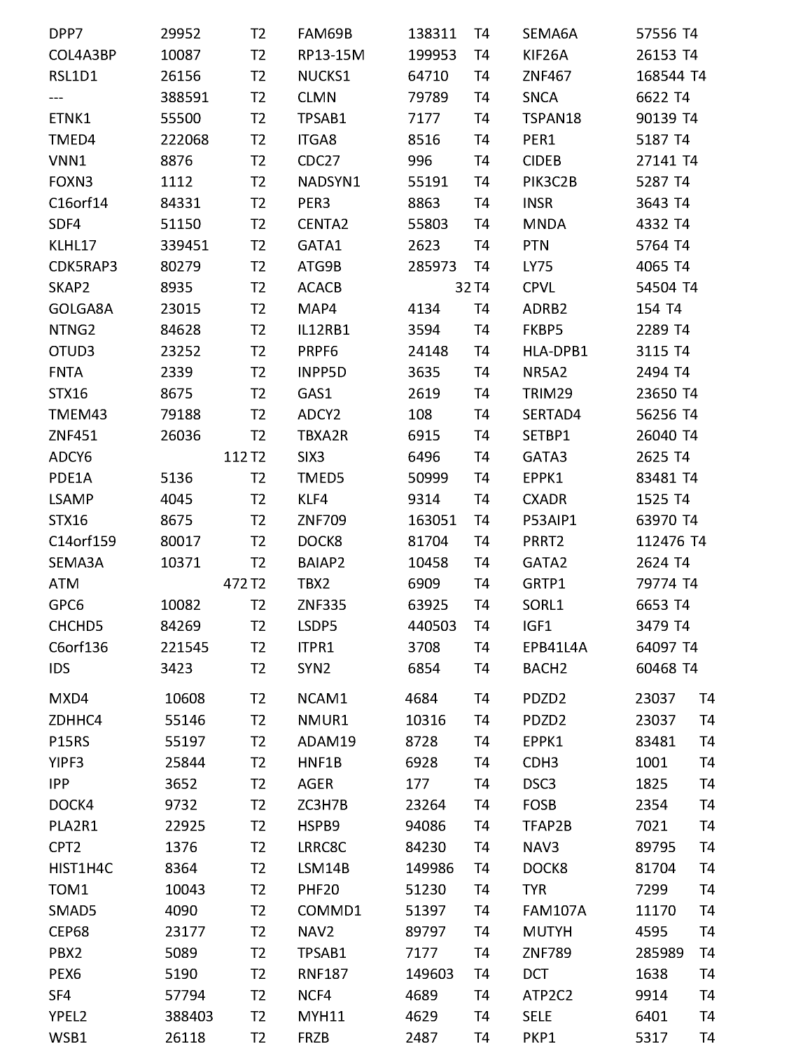

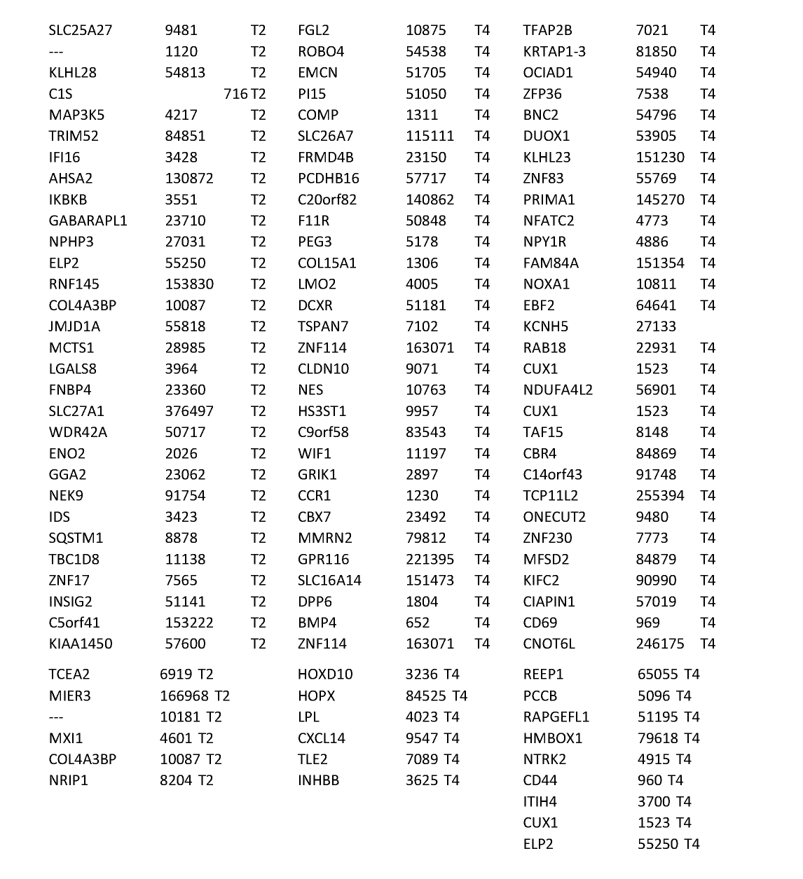

Pre-existing microarray data from JAK-inhibitor experiments (1,2) were re-analyzed and JAK inhibition produced a consistent downregulation of OSM-Rβ whole mouse skin treated during telogen (FIG. 11A). Downregulation of OSM-Rβ This was confirmed with qRT-PCR in vivo, where whole skin biopsies were collected daily over a period of 5 days of Tofacitinib or Ruxolitinib treatment (FIG. 11B). Downregulation of OSM-Rβ transcription was also found in vitro in cultured mouse keratinocytes (FIG. 11C). Sorted keratinocytes from telogen mouse skin treated with Ruxolitinib also exhibited a downregulation of OSM-Rβ after 3 days (FIG. 11D). In vivo treatment with Ruxolitinib, a JAK2 inhibitor, led to early STAT3/5 inhibition, then an activation of Akt and ERK pathways, leading to a downregulation of OSM-Rβ (FIG. 11E). The data suggest that OSM produced in the dermal compartment, possibly the dermal papilla, engages OSM-Rβ on the HFSCs in the bulge to maintain quiescence during telogen by suppressing growth and differentiation, at the same time maintaining OSM-Rβ expression. JAK inhibition likely breaks this positive feedback loop and sends the hair follicle into anagen.

### REFERENCES

1. Harel S, Higgins C A, Cerise J E, et al. Science Adv 1(9), October 2015
2. Xing L, Dai Z, Jabbari A et al., Nat Med 20, September 2014
3. Blau H M, Cosgrove B D, Ho A T. Nat Med 21(8), August 2015
4. Doles J, Storer M, Cozzuto L, et al. Genes Development 26(19), October 2012
5. Woo S H, Stumpfova M, Jensen U B, et al. Development 137(23), December 2010

Various publications, patents and patent application are cited herein, the contents of which are hereby incorporated by reference in their entireties.

We claim:

1. A method of inducing hair growth in a mammalian subject, the method comprising administering to a hair follicle of the subject a therapeutically effective amount of a Jak1, a Jak2, a Jak3, a Tyk2, a STAT1, a STAT2, a STAT3, a STAT4, a STAT5a, a STAT5b, a STAT6, an OSM, a gp130, an LIFR, and/or an OSM-Rβ inhibitor.

2. The method of claim 1, wherein said administration occurs when the hair follicle is in mid-telogen phase or late telogen phase.

3. The method of claim 1, wherein the subject has androgenetic alopecia, telogen effluvium, alopecia areata, tinea capitis, alopecia totalis, hypotrichosis, hereditary hypotrichosis simplex, frontal fibrosing alopecia, cicatricial alopecia, lichen planopilaris, ring alopecia, scarring alopacia, nonscarring alopacia, chemotherapy induced alopecia, or alopecia universalis.

4. The method of claim 1, wherein the inhibitor is an antisense RNA, an siRNA, an shRNA, a microRNA, or a variant or modification thereof that specifically inhibits expression of the gene that encodes the Jak1, Jak2, Jak3, Tyk2, STAT1, STAT2, STAT3, STAT4, STAT5a, STAT5b, STAT6, OSM, gp130, LIFR, or OSM-Rβ; or a small molecule.

5. The method of claim 4, wherein the inhibitor is ruxolitinib (INCB 018424).

6. The method of claim 4, wherein the inhibitor is tofacitinib (CP690550).

7. The method of claim 4, wherein the small molecule is ruxolitinib (INCB 018424), tofacitinib (CP690550), AG490, momelotinib (CYT387), partcitinib (SB1518), baricitinib (LY3009104), fedratinib (TG101348), BMS-911543, lestaurtinib (CEP-701), fludarabine, epigallocatechin-3-gallate (EGCG), baricitinib, momelotinib, pacritinib, peficitinib, ABT 494, AT 9283, decernmotinib, filgotinib, gandotinib, INCB 39110, PF 4965842, R348, AZD 1480, BMS 911543, cerdulatinib, INCB 052793, NS 018, C 410, CT 1578, JTE 052, PF 6263276, R 548, TG 02, lumbricus rebellus extract, ARN 4079, AR 13154, UR 67767, CS510, VR588, DNX 04042, or hyperforin or combinations thereof.

8. The method of claim 4, wherein the inhibitor is an OSM-Rβ antibody.

9. The method of claim 1, wherein the subject is a human.

10. The method of claim 1, wherein the hair is on a scalp or a face, or constitutes an eyebrow or an eyelash of the subject.

11. The method of claim 1, wherein the hair is nasal hair.

12. The method of claim 1, wherein the inhibitor is administered topically.

13. The method of claim 1, wherein the inhibitor is administered orally.

14. The method of claim 1, wherein an expression level of one or more hair growth biomarkers are changed after administering said inhibitor.

15. The method of claim 14, wherein the one or more hair growth biomarkers are selected from the group consisting of CD34, Lhx2, NFATc1, Axin2, FoxC1, OSMR, OSM, Jak3, FAS, Irf1, Ifnar1, Nr3c1, Stat5A, Il6st, Ptprc, Ghr, IL10ra, Il2rg, Pdgfra, Spfi1, Socs2, Stat5b, Crp, Il4, Prlr, Insr, IL2ra, Cebpd, Stat3, Jak1, Acvr2a, Sfrp4, Sox5, Cdh2, Fzd5, Wif1, Wnt2, Fzd8, Apc, Sox9, Ilk, Shh, Krt25, Dlx2, Prom1, S100a9, Vegfc, Ptgfr, Pdgfr1, Igfbp4, Gli2, Tyrp1, Syt4, Mlana, Pme1, Dct, Tyr, Sos1, Dbf4, Pax3, PIK3ca, Rps6kb1, Mlph, and Stx17.

16. The method of claim 14, wherein the gene expression change of one or more biomarkers are detected by quantitative PCR or a variation thereof.

17. The method of claim 14, wherein the gene expression change of one or more biomarkers are detected by enzyme linked immunosorbent assay or a variation thereof.

18. A method of promoting hair growth in a mammalian subject, the method comprising administering to a hair follicle of the subject a therapeutically effective amount of a Jak1, a Jak2, a Jak3, a Tyk2, a STAT1, a STAT2, a STAT3, a STAT4, a STAT5a, a STAT5b, a STAT6, an OSM, a gp130, an LIFR, and/or an OSM-Rβ inhibitor.

19. The method of claim 18, wherein said administration occurs when the hair follicle is in a phase other than early telogen phase.

20. The method of claim 19, wherein the hair follicle is in anagen phase.

21. The method of claim 18, wherein the subject has androgenetic alopecia, telogen effluvium, alopecia areata, tinea capitis, alopecia totalis, hypotrichosis, hereditary hypotrichosis simplex, frontal fibrosing alopecia, cicatricial alopecia, lichen planopilaris, ring alopecia, scarring alopacia, nonscarring alopacia, chemotherapy induced alopecia, or alopecia universalis.

22. The method of claim 18, wherein the inhibitor is an antisense RNA, an siRNA, an shRNA, a microRNA, or a variant or modification thereof that specifically inhibits expression of the gene that encodes the Jak1, Jak2, Jak3, Tyk2, STAT1, STAT2, STAT3, STAT4, STAT5a, STAT5b, STAT6, OSM, gp130, LIFR, or OSM-Rβ; or a small molecule.

23. The method of claim 22, wherein the inhibitor is ruxolitinib (INCB 018424).

24. The method of claim 22, wherein the inhibitor is tofacitinib (CP690550).

25. The method of claim 22, wherein the small molecule is ruxolitinib (INCB 018424), tofacitinib (CP690550), AG490, momelotinib (CYT387), partcitinib (SB1518), baricitinib (LY3009104), fedratinib (TG101348), BMS-911543, lestaurtinib (CEP-701), fludarabine, epigallocatechin-3-gallate (EGCG), baricitinib, momelotinib, pacritinib, peficitinib, ABT 494, AT 9283, decernmotinib, filgotinib, gandotinib, INCB 39110, PF 4965842, R348, AZD 1480, BMS 911543, cerdulatinib, INCB 052793, NS 018, C 410, CT 1578, JTE 052, PF 6263276, R 548, TG 02, lumbricus rebellus extract, ARN 4079, AR 13154, UR 67767, CS510, VR588, DNX 04042, or hyperforin or combinations thereof.

26. The method of claim 22, wherein the inhibitor is an OSM-Rβ antibody.

27. The method of claim 18, wherein the subject is a human.

28. The method of claim 18, wherein the hair is on a scalp or a face, or constitutes an eyebrow or an eyelash of the subject.

29. The method of claim 18, wherein the hair is nasal hair.

30. The method of claim 18, wherein the inhibitor is administered topically.

31. The method of claim 18, wherein the inhibitor is administered orally.

32. The method of claim 18, wherein an expression level of one or more hair growth biomarkers are changed after administering said inhibitor.

33. The method of claim 32, wherein the one or more biomarkers are selected from the group consisting of CD34, Lhx2, NFATc1, Axin2, FoxC1, OSMR, OSM, Jak3, FAS, Irf1, Ifnar1, Nr3c1, Stat5A, Il6st, Ptprc, Ghr, IL10ra, Il2rg, Pdgfra, Spfi1, Socs2, Stat5b, Crp, Il4, Prlr, Insr, IL2ra, Cebpd, Stat3, Jak1, Acvr2a, Sfrp4, Sox5, Cdh2, Fzd5, Wif1, Wnt2, Fzd8, Apc, Sox9, Ilk, Shh, Krt25, Dlx2, Prom1, S100a9, Vegfc, Ptgfr, Pdgfr1, Igfbp4, Gli2, Tyrp1, Syt4, Mlana, Pme1, Dct, Tyr, Sos1, Dbf4, Pax3, PIK3ca, Rps6kb1, Mlph, and Stx17.

34. The method of claim 32, wherein the gene expression change of one or more biomarkers are detected by quantitative PCR or a variation thereof.

35. The method of claim 32, wherein the gene expression change of one or more biomarkers are detected by enzyme linked immunosorbent assay or a variation thereof.

36. A method of promoting inductivity of dermal papilla, the method comprising administering to a dermal papilla 3D sphere derived from a hair follicle of a subject a therapeutically effective amount of a Jak1, a Jak2, a Jak3, a Tyk2, a STAT1, a STAT2, a STAT3, a STAT4, a STAT5a, a STAT5b, a STAT6, an OSM, a gp130, an LIFR, and/or an OSM-Rβ inhibitor, wherein said administration occurs before administering said dermal papilla spheres to a subject.

37. The method of claim 36, wherein the dermal papilla 3D sphere is subsequently administered to the subject to treat androgenetic alopecia, telogen effluvium, alopecia areata, tinea capitis, alopecia totalis, hypotrichosis, hereditary hypotrichosis simplex, frontal fibrosing alopecia, cicatricial alopecia, lichen planopilaris, ring alopecia, scarring alopacia, nonscarring alopacia, chemotherapy induced alopecia, or alopecia universalis.

38. The method of claim 36, wherein the inhibitor is an antisense RNA, an siRNA, an shRNA, a microRNA, or a variant or modification thereof that specifically inhibits expression of the gene that encodes the Jak1, Jak2, Jak3, Tyk2, STAT1, STAT2, STAT3, STAT4, STAT5a, STAT5b, STAT6, OSM, gp130, LIFR, or OSM-Rβ; or a small molecule.

39. The method of claim 38, wherein the inhibitor is ruxolitinib (INCB 018424).

40. The method of claim 38, wherein the inhibitor is tofacitinib (CP690550).

41. The method of claim 38, wherein the small molecule is ruxolitinib (INCB 018424), tofacitinib (CP690550), AG490, momelotinib (CYT387), partcitinib (SB1518), baricitinib (LY3009104), fedratinib (TG101348), BMS-911543, lestaurtinib (CEP-701), fludarabine, epigallocatechin-3-gallate (EGCG), baricitinib, momelotinib, pacritinib, peficitinib, ABT 494, AT 9283, decernmotinib, filgotinib, gandotinib, INCB 39110, PF 4965842, R348, AZD 1480, BMS 911543, cerdulatinib, INCB 052793, NS 018, C 410, CT 1578, JTE 052, PF 6263276, R 548, TG 02, lumbricus rebellus extract, ARN 4079, AR 13154, UR 67767, CS510, VR588, DNX 04042, or hyperforin or combinations thereof.

42. The method of claim 38, wherein the inhibitor is an OSM-Rβ antibody.

43. The method of claim 36, wherein the subject is a human.

44. The method of claim 36, wherein the hair is on a scalp or a face, or constitutes an eyebrow or an eyelash of the subject.

45. The method of claim 36, wherein the hair is nasal hair.

46. The method of claim 36, wherein the inhibitor is administered topically.

47. The method of claim 36, wherein the inhibitor is administered orally.

48. The method of claim 36, wherein an expression level of one or more hair growth biomarkers are changed after administering said inhibitor.

49. The method of claim 48, wherein the one or more biomarkers are selected from the group consisting of CD34, Lhx2, NFATc1, Axin2, FoxC1, OSMR, OSM, Jak3, FAS, Irf1, Ifnar1, Nr3c1, Stat5A, Il6st, Ptprc, Ghr, IL10ra, Il2rg, Pdgfra, Spfi1, Socs2, Stat5b, Crp, Il4, Prlr, Insr, IL2ra, Cebpd, Stat3, Jak1, Acvr2a, Sfrp4, Sox5, Cdh2, Fzd5, Wif1, Wnt2, Fzd8, Apc, Sox9, Ilk, Shh, Krt25, Dlx2, Prom1, S100a9, Vegfc, Ptgfr, Pdgfr1, Igfbp4, Gli2, Tyrp1, Syt4, Mlana, Pme1, Dct, Tyr, Sos1, Dbf4, Pax3, PIK3ca, Rps6kb1, Mlph, and Stx17.

50. The method of claim 48, wherein the gene expression change of one or more biomarkers are detected by quantitative PCR or a variation thereof.

51. The method of claim 48, wherein the gene expression change of one or more biomarkers are detected by enzyme linked immunosorbent assay or a variation thereof.

52. A method of assessing the efficacy of a therapy for inducing or promoting hair growth in a mammalian subject, the method comprising:
(a) determining a level of one or more hair growth biomarkers in a hair follicle sample obtained from the subject; and
(b) determining the level of the one or more hair growth biomarkers in a hair follicle sample obtained from the subject, at one of more time points during the therapy,
wherein the therapy is efficacious for inducing or promoting hair growth in the subject when there is a change of the one or more biomarkers in the second or subsequent samples, relative to the first sample.

53. The method of claim 52, wherein the one or more biomarkers are selected from the group consisting of CD34, Lhx2, NFATc1, Axin2, FoxC1, OSMR, OSM, Jak3, FAS, Irf1, Ifnar1, Nr3c1, Stat5A, Il6st, Ptprc, Ghr, IL10ra, Il2rg, Pdgfra, Spfi1, Socs2, Stat5b, Crp, Il4, Prlr, Insr, IL2ra, Cebpd, Stat3, Jak1, Acvr2a, Sfrp4, Sox5, Cdh2, Fzd5, Wif1, Wnt2, Fzd8, Apc, Sox9, Ilk, Shh, Krt25, Dlx2, Prom1, S100a9, Vegfc, Ptgfr, Pdgfr1, Igfbp4, Gli2, Tyrp1, Syt4, Mlana, Pme1, Dct, Tyr, Sos1, Dbf4, Pax3, PIK3ca, Rps6kb1, Mlph, and Stx17.

54. The method of claim 52, wherein the gene expression change of one or more biomarkers are detected by quantitative PCR or a variation thereof.

55. The method of claim 52, wherein the gene expression change of one or more biomarkers are detected by enzyme linked immunosorbent assay or a variation thereof.

56. A kit for inducing or promoting hair growth in a mammalian subject, the kit comprising:
(a) a Jak1, a Jak2, a Jak3, a Tyk2, a STAT1, a STAT2, a STAT3, a STAT4, a STAT5a, a STAT5b, a STAT6, an OSM, a gp130, an LIFR, and/or an OSM-Rβ inhibitor; and
(b) a pharmaceutically acceptable carrier.

57. The kit of claim 56, wherein the inhibitor is an antisense RNA, an siRNA, an shRNA, a microRNA, or a variant or modification thereof that specifically inhibits expression of the gene that encodes the Jak1, Jak2, Jak3, Tyk2, STAT1, STAT2, STAT3, STAT4, STAT5a, STAT5b, STAT6, OSM, gp130, LIFR, or OSM-Rβ; or a small molecule.

58. The kit of claim 57, wherein the inhibitor is ruxolitinib (INCB 018424).

59. The kit of claim 57, wherein the inhibitor is tofacitinib (CP690550).

60. The kit of claim 57, wherein the small molecule is ruxolitinib (INCB 018424), tofacitinib (CP690550), AG490, momelotinib (CYT387), partcitinib (SB1518), baricitinib (LY3009104), fedratinib (TG101348), BMS-911543, lestaurtinib (CEP-701), fludarabine, epigallocatechin-3-gallate (EGCG), baricitinib, momelotinib, pacritinib, peficitinib, ABT 494, AT 9283, decernmotinib, filgotinib, gandotinib, INCB 39110, PF 4965842, R348, AZD 1480, BMS 911543, cerdulatinib, INCB 052793, NS 018, C 410, CT 1578, JTE 052, PF 6263276, R 548, TG 02, lumbricus rebellus extract, ARN 4079, AR 13154, UR 67767, CS510, VR588, DNX 04042, or hyperforin or combinations thereof.

61. The method of claim 57, wherein the inhibitor is an OSM-Rβ antibody.


SEQUENCE LISTING (11 sequences)

SEQ ID NO: 1 - DNA, 21 bp, Artificial Sequence
  Description of Artificial Sequence Synthetic primer
ttctggtgcttgtctcactga

SEQ ID NO: 2 - DNA, 22 bp, Artificial Sequence
  Description of Artificial Sequence Synthetic primer
cagtatgttcggcttcccattc

SEQ ID NO: 3 - DNA, 20 bp, Artificial Sequence
  Description of Artificial Sequence Synthetic primer
caccatgcttttgtggacag

SEQ ID NO: 4 - DNA, 18 bp, Artificial Sequence
  Description of Artificial Sequence Synthetic primer
gttactgcgactggtgcg

SEQ ID NO: 5 - DNA, 21 bp, Artificial Sequence
  Description of Artificial Sequence Synthetic primer
agaaggtccatacagtggaag

SEQ ID NO: 6 - DNA, 19 bp, Artificial Sequence
  Description of Artificial Sequence Synthetic primer
aagcaggtttcgactgggt

SEQ ID NO: 7 - DNA, 20 bp, Artificial Sequence
  Description of Artificial Sequence Synthetic primer
ccgggacgtaagtccttcac

SEQ ID NO: 8 - DNA, 20 bp, Artificial Sequence
  Description of Artificial Sequence Synthetic primer
caacgcctactctcccagac

SEQ ID NO: 9 - DNA, 22 bp, Artificial Sequence
  Description of Artificial Sequence Synthetic primer
gagccttgatgtactgtaccac

SEQ ID NO: 10 - DNA, 21 bp, Artificial Sequence
  Description of Artificial Sequence Synthetic primer
atggtcacccacagcaagttt

SEQ ID NO: 11 - DNA, 21 bp, Artificial Sequence
  Description of Artificial Sequence Synthetic primer
tccagtagaatccgctctcct